SEQUENCE LISTING (91 sequences)

SEQ ID NO: 1 - protein, 1296 aa, Clostridium botulinum Serotype A
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 2 - protein, 1291 aa, Clostridium botulinum Serotype B
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 3 - protein, 1291 aa, Clostridium botulinum Serotype C1
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 4 - protein, 1276 aa, Clostridium botulinum Serotype D
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 5 - protein, 1252 aa, Clostridium botulinum Serotype E
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 6 - protein, 1274 aa, Clostridium botulinum Serotype F
MPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRKGTKAPPRLCIRVNNSELFFVASESSYNENDINTPKEIDDTTNLNNNYRNNLDEVILDYNSQTIPQISNRTLNTLVQDNSYVPRYDSNGTSEIEEYDVVDFNVFFYLHAQKVPEGETNISLTSSIDTALLEESKDIFFSSEFIDTINKPVNAALFIDWISKVIRDFTTEATQKSTVDKIADISLIVPYVGLALNIIIEAEKGNFEEAFELLGVGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESEYNINNIEEELNKKVSLAMKNIERFMTESSISYLMKLINEAKVGKLKKYDNHVKSDLLNYILDHRSILGEQTNELSDLVTSTLNSSIPFELSSYTNDKILIIYFNRLYKKIKDSSILDMRYENNKFIDISGYGSNISINGNVYIYSTNRNQFGIYNSRLSEVNIAQNNDIIYNSRYQNFSISFWVRIPKHYKPMNHNREYTIINCMGNNNSGWKISLRTVRDCEIIWTLQDTSGNKENLIFRYEELNRISNYINKWIFVTITNNRLGNSRIYINGNLIVEKSISNLGDIHVSDNILFKIVGCDDETYVGIRYFKVFNTELDKTEIETLYSNEPDPSILKNYWGNYLLYNKKYYLFNLLRKDKYITLNSGILNINQQRGVTEGSVFLNYKLYEGVEVIIRKNGPIDISNTDNFVRKNDLAYINVVDRGVEYRLYADTKSEKEKIIRTSNLNDSLGQIIVMDSIGNNCTMNFQNNNGSNIGLLGFHSNNLVASSWYYNNIRRNTSSNGCFWSSISKENGWKE

SEQ ID NO: 7 - protein, 1297 aa, Clostridium botulinum Serotype G
MPVNIKNFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 8 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 9 - protein, 1268 aa, Clostridium baratii
MPVNINNFNYNDPINNTTILYMKMPYYEDSNKYYKAFEIMDNVWIIPERNIIGKKPSDFYPPISLDSGSSAYYDPNYLTTDAEKDRFLKTVIKLFNRINSNPAGQVLLEEIKNGKPYLGNDHTAVNEFCANNRSTSVEIKESNGTTDSMLLNLVILGPGPNILECSTFPVRIFPNNIAYDPSEKGFGSIQLMSFSTEYEYAFNDNTDLFIADPAISLAHELIHVLHGLYGAKGVTNKKVIEVDQGALMAAEKDIKIEEFITFGGQDLNIITNSTNQKIYVILLSNYTAIASRLSQVNRNNSALNTTYYKNFFQWKYGLDQDSNGNYTVNISKFNAIYKKLFSFTECDLAQKFQVKNRSNYLFHFKPFRLLDLLDDNIYSISEGFNIGSLRVNNNGQNINLNSRIVGPIPDNGLVERFVGLCKSIVSKKGTKNSLCIKVNNRDLFFVASESSYNENGINSPKEIDDTTITNNNYKKNLDEVILDYNSDAIPNLSSRLLNTTAQNDSYVPKYDSNGTSEIKEYTVDKLNVFFYLYAQKAPEGESAISLTSSVNTALLDASKVYTFFSSDFINTVNKPVQAALFISWIQQVINDFTTEATQKSTIDKIADISLIVPYVGLALNIGNEVQKGNFKEAIELLGAGILLEFVPELLIPTILVFTIKSFINSDDSKNKIIKAINNALRERELKWKEVYSWIVSNWLTRINTQFNKRKEQMYQALQNQVDGIKKIIEYKYNNYTLDEKNRLRAEYNIYSIKEELNKKVSLAMQNIDRFLTESSISYLMKLINEAKINKLSEYDKRVNQYLLNYILENSSTLGTSSVPELNNLVSNTLNNSIPFELSEYTNDKILIHILIRFYKRIIDSSILNMKYENNRFIDSSGYGSNISINGDIYIYSTNRNQFGIYSSRLSEVNITQNNTIIYNSRYQNFSVSFWVRIPKYNNLKNLNNEYTIINCMRNNNSGWKISLNYNNIIWTLQDTTGNNQKLVFNYTQMIDISDYINKWTFVTITNNRLGHSKLYINGNLTDQKSILNLGNIHVDDNILFKIVGCNDTRYVGIRYFKIFNMELDKTEIETLYHSEPDSTILKDFWGNYLLYNKKYYLLNLLKPNMSVTKNSDILNINRQRGIYSKTNIFSNARLYTGVEVIIRKVGSTDTSNTDNFVRKNDTVYINVVDGNSEYQLYADVSTSAVEKTIKLRRISNSNYNSNQMIIMDSIGDNCTMNFKTNNGNDIGLLGFHLNNLVASSWYYKNIRNNTRNNGCFWSFISKEHGWQE

SEQ ID NO: 10 - protein, 1251 aa, Clostridium butyricum
MPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVGWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKAIIESKYNSYTLEEKNELTNKYDIEQIENELNQKVSIAMNNIDRFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNSGINQKLAFNYGNANGISDYINKWIFVTITNDRLGDSKLYINGNLIDKKSILNLGNIHVSDNILFKIVNCSYTRYIGIRYFNIFDKELDETEIQTLYNNEPNANILKDFWGNYLLYDKEYYLLNVLKPNNFINRRTDSTLSINNIRSTILLANRLYSGIKVKIQRVNNSSTNDNLVRKNDQVYINFVASKTHLLPLYADTATTNKEKTIKISSSGNRFNQVVVMNSVGNCTMNFKNNNGNNIGLLGFKADTVVASTWYYTHMRDNTNSNGFFWNFISEEHGWQEK

SEQ ID NO: 11 - protein, 25 aa, Artificial Sequence
  BoNT/A di-chain loop region
CVRGIITSKTKSLDKGYNKALNDLC

SEQ ID NO: 12 - protein, 10 aa, Artificial Sequence
  BoNT/B di-chain loop region
CKSVKAPGIC

SEQ ID NO: 13 - protein, 17 aa, Artificial Sequence
  BoNT/C1 di-chain loop region
CHKAIDGRSLYNKTLDC

SEQ ID NO: 14 - protein, 14 aa, Artificial Sequence
  BoNT/D di-chain loop region
CLRLTKNSRDDSTC

SEQ ID NO: 15 - protein, 15 aa, Artificial Sequence
  BoNT/E di-chain loop region
CKNIVSVKGIRKSIC

SEQ ID NO: 16 - protein, 17 aa, Artificial Sequence
  BoNT/F di-chain loop region
CKSVIPRKGTKAPPRLC

SEQ ID NO: 17 - protein, 15 aa, Artificial Sequence
  BoNT/G di-chain loop region
CKPVMYKNTGKSEQC

SEQ ID NO: 18 - protein, 29 aa, Artificial Sequence
  TeNT di-chain loop region
CKKIIPPTNIRENLYNRTASLTDLGGELC

SEQ ID NO: 19 - protein, 15 aa, Artificial Sequence
  BaNT di-chain loop region
CKSIVSKKGTKNSLC

SEQ ID NO: 20 - protein, 15 aa, Artificial Sequence
  BuNT di-chain loop region
CKNIVSVKGIRKSIC

SEQ ID NO: 21 - protein, 5 aa, Artificial Sequence
  Bovine enterokinase protease cleavage site
DDDDK

SEQ ID NO: 22 - protein, 7 aa, Artificial Sequence
  Tobacco Etch Virus protease cleavage site consensus sequence
EXXYXQG

SEQ ID NO: 23 - protein, 7 aa, Artificial Sequence
  Tobacco Etch Virus protease cleavage site consensus sequence
EXXYXQS

SEQ ID NO: 24 - protein, 7 aa, Artificial Sequence
  Tobacco Etch Virus protease cleavage site
ENLYFQG

SEQ ID NO: 25 - protein, 7 aa, Artificial Sequence
  Tobacco Etch Virus protease cleavage site
ENLYFQS

SEQ ID NO: 26 - protein, 7 aa, Artificial Sequence
  Tobacco Etch Virus protease cleavage site
ENIYTQG

SEQ ID NO: 27 - protein, 7 aa, Artificial Sequence
  Tobacco Etch Virus protease cleavage site
ENIYTQS

SEQ ID NO: 28 - protein, 7 aa, Artificial Sequence
  Tobacco Etch Virus protease cleavage site
ENIYLQG

SEQ ID NO: 29 - protein, 7 aa, Artificial Sequence
  Tobacco Etch Virus protease cleavage site
ENIYLQS

SEQ ID NO: 30 - protein, 7 aa, Artificial Sequence
  Tobacco Etch Virus protease cleavage site
ENVYFQG

SEQ ID NO: 31 - protein, 7 aa, Artificial Sequence
  Tobacco Etch Virus protease cleavage site
ENVYSQS

SEQ ID NO: 32 - protein, 7 aa, Artificial Sequence
  Tobacco Etch Virus protease cleavage site
ENVYSQG

SEQ ID NO: 33 - protein, 7 aa, Artificial Sequence
  Tobacco Etch Virus protease cleavage site
ENVYSQS

SEQ ID NO: 34 - protein, 7 aa, Artificial Sequence
  Tobacco Vein Mottling Virus protease cleavage site consensus sequence
XXVRFQG

SEQ ID NO: 35 - protein, 7 aa, Artificial Sequence
  Tobacco Vein Mottling Virus protease cleavage site consensus sequence
XXVRFQS

SEQ ID NO: 36 - protein, 7 aa, Artificial Sequence
  Tobacco Vein Mottling Virus protease cleavage site
ETVRFQG

SEQ ID NO: 37 - protein, 7 aa, Artificial Sequence
  Tobacco Vein Mottling Virus protease cleavage site
ETVRFQS

SEQ ID NO: 38 - protein, 7 aa, Artificial Sequence
  Tobacco Vein Mottling Virus protease cleavage site
NNVRFQG

SEQ ID NO: 39 - protein, 7 aa, Artificial Sequence
  Tobacco Vein Mottling Virus protease cleavage site
NNVRFQS

SEQ ID NO: 40 - protein, 7 aa, Artificial Sequence
  Human Rhinovirus 3C protease cleavage site consensus sequence
XXLFQGP

SEQ ID NO: 41 - protein, 7 aa, Artificial Sequence
  Human Rhinovirus 3C protease cleavage site
EALFQGP

SEQ ID NO: 42 - protein, 7 aa, Artificial Sequence
  Human Rhinovirus 3C protease cleavage site
EVLFQGP

SEQ ID NO: 43 - protein, 7 aa, Artificial Sequence
  Human Rhinovirus 3C protease cleavage site
ELLFQGP

SEQ ID NO: 44 - protein, 7 aa, Artificial Sequence
  Human Rhinovirus 3C protease cleavage site
DALFQGP

SEQ ID NO: 45 - protein, 7 aa, Artificial Sequence
  Human Rhinovirus 3C protease cleavage site
DVLFQGP

SEQ ID NO: 46 - protein, 7 aa, Artificial Sequence
  Human Rhinovirus 3C protease cleavage site
DLLFQGP

SEQ ID NO: 47 - protein, 6 aa, Artificial Sequence
  Subtilisin cleavage site consensus sequence
XXXXHY

SEQ ID NO: 48 - protein, 6 aa, Artificial Sequence
  Subtilisin cleavage site consensus sequence
XXXXYH

SEQ ID NO: 49 - protein, 2 aa, Artificial Sequence
  Subtilisin cleavage site
HY

SEQ ID NO: 50 - protein, 2 aa, Artificial Sequence
  Subtilisin cleavage site
YH

SEQ ID NO: 51 - protein, 6 aa, Artificial Sequence
  Subtilisin cleavage site
PGAAHY

SEQ ID NO: 52 - protein, 6 aa, Artificial Sequence
  Hydroxylamine cleavage site
NGNGNG

SEQ ID NO: 53 - protein, 2 aa, Artificial Sequence
  Hydroxylamine cleavage site
NG

SEQ ID NO: 54 - protein, 5 aa, Artificial Sequence
  SUMO/ULP-1 protease cleavage site consensus sequence
GGXXX

SEQ ID NO: 55 - protein, 98 aa, Artificial Sequence
  SUMO/ULP-1 protease cleavage site
MADSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGG

SEQ ID NO: 56 - protein, 5 aa, Artificial Sequence
  Caspase 3 protease cleavage site consensus sequence
DXXDX

SEQ ID NO: 57 - protein, 5 aa, Artificial Sequence
  Caspase 3 protease cleavage site
DEVDG

SEQ ID NO: 58 - protein, 5 aa, Artificial Sequence
  Caspase 3 protease cleavage site
DEVDS

SEQ ID NO: 59 - protein, 5 aa, Artificial Sequence
  Caspase 3 protease cleavage site
DEPDG

SEQ ID NO: 60 - protein, 5 aa, Artificial Sequence
  Caspase 3 protease cleavage site
DEPDS

SEQ ID NO: 61 - protein, 5 aa, Artificial Sequence
  Caspase 3 protease cleavage site
DELDG

SEQ ID NO: 62 - protein, 5 aa, Artificial Sequence
  Caspase 3 protease cleavage site
DELDS

SEQ ID NO: 63 - protein, 4 aa, Artificial Sequence
  Flexible G-spacer
GGGG

SEQ ID NO: 64 - protein, 5 aa, Artificial Sequence
  Flexible G-spacer
GGGGS

SEQ ID NO: 65 - protein, 4 aa, Artificial Sequence
  Flexible A-spacer
AAAA

SEQ ID NO: 66 - protein, 5 aa, Artificial Sequence
  Flexible A-spacer
AAAAV

SEQ ID NO: 67 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 68 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 69 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 70 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 71 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 72 - protein, 199 aa, Homo sapiens [sequence omitted]

SEQ ID NO: 73 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 74 - protein, 207 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 75 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 76 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 77 - protein, 345 aa, Homo sapiens
MSLFGLLLLTSALAGQRQGTQAESNLSSKFQFSSNKEQNGVQDPQHERIITVSTNGSIHSPRFPHTYPRNTVLVWRLVAVEENVWIQLTFDERFGLEDPEDDICKYDFVEVEEPSDGTILGRWCGSGTVPGKQISKGNQIRIRFVSDEYFPSEPGFCIHYNIVMPQFTEAVSPSVLPPSALPLDLLNNAITAFSTLEDLIRYLEPERWQLDLEDLYRPTWQLLGKAFVFGRKSRVVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEECDCVCRGSTGG

SEQ ID NO: 78 - protein, 221 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRHSPGRQSPDMPGDFRADAPSFLPPRRSLPMLFRMEWGCALTGSQSAVWPSSPVPEEIPRMHPGRNGKKQQRKPLREKMKPERCGDAVPRR

SEQ ID NO: 79 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 80 - protein, 180 aa, Homo sapiens
MGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 81 - protein, 1207 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVIRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGVHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 82 - protein, 208 aa, Homo sapiens
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTTILAVVAVVLSSVCLLVIVGLLMFRYHRRGGYDVENEEKVKLGMTNSH

SEQ ID NO: 83 - protein, 160 aa, Homo sapiens
MVPSAGQLALFALGIVLAACQALENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 84 - protein, 252 aa, Homo sapiens
MRAPLLPPAPVVLSLLILGSGHYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSKIALAAIAAFMSAVILTAVAVITVQLRRQYVRKYEGEAEERKKLRQENGNVHAIA

SEQ ID NO: 85 - protein, 169 aa, Homo sapiens
MTAGRRMEMLCAGRVPALLLCLGFHLLQAVLSTTVIPSCIPGESSDNCTALVQTEDNPRVAQVSITKCSSDMNGYCLHGQCIYLVDMSQNYCRCEVGYTGVRCEHFFLTVHQPLSKEYVALTVILIILFLITVVGSTYYFCRWYRNRKSKEPKKEYERVTSGDPELPQV

SEQ ID NO: 86 - protein, 154 aa, Homo sapiens
MALGVPISVYLLFNAMTALTEEAAVTVTPPITAQQGNWTVNKTEADNIEGPIALKFSHLCLEDHNSYCINGACAFHHELEKAICRCFTGYTGERCEHLTLTSYAVDSYEKYIAIGIGVGLLLSGFLVIFYCYIRKRCLKLKSPYNVCSGERRPL

SEQ ID NO: 87 - protein, 178 aa, Homo sapiens
MDRAARCSGASSLPLLLALALGLVILHCVVADGNSTRSPETNGLLCGDPEENCAATTTQSKRKGHFSRCPKQYKHYCIKGRCRFVVAEQTPSCVCDEGYIGARCERVDLFYLRGDRGQILVICLIAVMVVFIILVIGVCTCCHPLRKRRKRKKKEEEMETLGKDITPINEDIEETNIA

SEQ ID NO: 88 - protein, 640 aa, Homo sapiens [sequence omitted]

SEQ ID NO: 89 - protein, 850 aa, Homo sapiens
MRQVCCSALPPPPLEKGRCSSYSDSSSSSSERSSSSSSSSSESGSSSRSSSNNSSISRPAAPPEPRPQQQPQPRSPAARRAAARSRAAAAGGMRRDPAPGFSMLLFGVSLACYSPSLKSVQDQAYKAPVVVEGKVQGLVPAGGSSSNSTREPPASGRVALVKVLDKWPLRSGGLQREQVISVGSCVPLERNQRYIFFLEPTEQPLVFKTAFAPLDTNGKNLKKEVGKILCTDCATRPKLKKMKSQTGQVGEKQSLKCEAAAGNPQPSYRWFKDGKELNRSRDIRIKYGNGRKNSRLQFNKVKVEDAGEYVCEAENILGKDTVRGRLYVNSVSTTLSSWSGHARKCNETAKSYCVNGGVCYYIEGINQLSCKCPNGFFGQRCLEKLPLRLYMPDPKQKAEELYQKRVLTITGICVALLVVGIVCVVAYCKTKKQRKQMHNHLRQNMCPAHQNRSLANGPSHPRLDPEEIQMADYISKNVPATDHVIRRETETTFSGSHSCSPSHHCSTATPTSSHRHESHTWSLERSESLTSDSQSGIMLSSVGTSKCNSPACVEARARRAAAYNLEERRRATAPPYHDSVDSLRDSPHSERYVSALTTPARLSPVDFHYSLATQVPTFEITSPNSAHAVSLPPAAPISYRLAEQQPLLRHPAPPGPGPGPGPGPGPGADMQRSYDSYYYPAAGPGPRRGTCALGGSLGSLPASPFRIPEDDEYETTQECAPPPPPRPRARGASRRTSAGPRRWRRSRLNGLAAQRARAARDSLSLSSGSGGGSASASDDDADDADGALAAESTPFLGLRGAHDALRSDSPPLCPAADSRTYYSLDSHSTRASSRHSRGPPPRAKQDSAPL

SEQ ID NO: 90 - protein, 696 aa, Homo sapiens
MSEGAAAASPPGAASAAAASAEEGTAAAAAAAAAGGGPDGGGEGAAEPPRELRCSDCIVWNRQQTWLCVVPLFIGFIGLGLSLMLLKWIVVGSVKEYVPTDLVDSKGMGQDPFFLSKPSSFPKAMETTTTTTSTTSPATPSAGGAASSRTPNRISTRLTTITRAPTRFPGHRVPIRASPRSTTARNTAAPATVPSTTAPFFSSSTLGSRPPVPGTPSTQAMPSWPTAAYATSSYLHDSTPSWTLSPFQDAASSSSSSSSSATTTTPETSTSPKFHTTTYSTERSEHFKPCRDKDLAYCLNDGECFVIETLTGSHKHCRCKEGYQGVRCDQFLPKTDSILSDPTDHLGIEFMESEEVYQRQVLSISCIIFGIVIVGMFCAAFYFKSKKQAKQIQEQLKVPQNGKSYSLKASSTMAKSENLVKSHVQLQNYSKVERHPVTALEKMMESSFVGPQSFPEVPSPDRGSQSVKHHRSLSSCCSPGQRSGMLHRNAFRRTPPSPRSRLGGIVGPAYQQLEESRIPDQDTIPCQGYSSSGLKTQRNTSINMQLPSRETNPYFNSLEQKDLVGYSSTRASSVPIIPSVGLEETCLQMPGISEVKSIKWCKNSYSADVVNVSIPVSDCLIAEQQEVKILLETVQEQIRILTDARRSEDYELASVETEDSASENTAFLPLSPTAKSEREAQFVLRNEIQRDSALTK

SEQ ID NO: 91 - protein, 115 aa, Homo sapiens
MPTDHEEPCGPSHKSFCLNGGLCYVIPTIPSPFCRCVENYTGARCEEVFLPGSSIQTKSNLFEAFVALAVLVTLIIGAFYFLCRKGHFQRASSVQYDINLVETSSTSAHHSHEQH

(19) United States
(12) Patent Application Publication
(10) Pub. No.: US 2010/0303757 A1
(43) Pub. Date: Dec. 2, 2010

(54) Methods of Treating Chronic Neurogenic Inflammation Using Interleukin Retargeted Endopepidases

(75) Inventors: JOSEPH FRANCIS, ALISO VIEJO, CA (US); DEAN G. STATHAKIS, IRVINE, CA (US)

Correspondence Address: ALLERGAN, INC., 2525 DUPONT DRIVE, T2-7H, IRVINE, CA, 92612-1599, US

(73) Assignee: ALLERGAN, INC.

(21) Appl. No.: 12/781,244

(22) Filed: May 17, 2010

Related U.S. Application Data
(60) Provisional application No. 61/182,224, filed on May 29, 2009.

(51) Int. Cl. A61K 38/20; A61K 38/43; C12N 9/96; A61P 29/00

(52) U.S. Cl. 424/85.2; 424/94.1; 435/188

(57) ABSTRACT

The present specification discloses TVEMPs, compositions comprising such toxins and methods of treating chronic neurogenic inflammation in a mammal using such TVEMPs and compositions.

### CROSS REFERENCE

This patent application claims priority pursuant to 35 U.S.C. §119(e) to U.S. Provisional Patent Application Ser. No. 61/182,224 filed May 29, 2009, which is hereby incorporated by reference in its entirety.

The ability of Clostridial toxins, such as, e.g., Botulinum neurotoxins (BoNTs), Botulinum neurotoxin serotype A (BoNT/A), Botulinum neurotoxin serotype B (BoNT/B), Botulinum neurotoxin serotype C1 (BoNT/C1), Botulinum neurotoxin serotype D (BoNT/D), Botulinum neurotoxin serotype E (BoNT/E), Botulinum neurotoxin serotype F (BoNT/F), and Botulinum neurotoxin serotype G (BoNT/G), and Tetanus neurotoxin (TeNT), to inhibit neuronal transmission are being exploited in a wide variety of therapeutic and cosmetic applications, see e.g., William J. Lipham, COSMETIC AND CLINICAL APPLICATIONS OF BOTULINUM TOXIN (Slack, Inc., 2004). Clostridial toxins commercially available as pharmaceutical compositions include, BoNT/A preparations, such as, e.g., BOTOX® (Allergan, Inc., Irvine, Calif.), DYSPORT®/RELOXIN®, (Beaufour Ipsen, Porton Down, England), NEURONOX® (Medy-Tox, Inc., Ochang-myeon, South Korea) BTX-A (Lanzhou Institute Biological Products, China) and XEOMIN® (Merz Pharmaceuticals, GmbH., Frankfurt, Germany); and BoNT/B preparations, such as, e.g., MYOBLOC™/NEUROBLOC™ (Elan Pharmaceuticals, San Francisco, Calif.). As an example, BOTOX® is currently approved in one or more countries for the following indications: achalasia, adult spasticity, anal fissure, back pain, blepharospasm, bruxism, cervical dystonia, essential tremor, glabellar lines or hyperkinetic facial lines, headache, hemifacial spasm, hyperactivity of bladder, hyperhidrosis, juvenile cerebral palsy, multiple sclerosis, myoclonic disorders, nasal labial lines, spasmodic dysphonia, strabismus and VII nerve disorder.

Clostridial toxin therapies are successfully used for many indications. Generally, administration of a Clostridial toxin treatment is well tolerated. However, toxin administration in some applications can be challenging because of the larger doses required to achieve a beneficial effect. Larger doses can increase the likelihood that the toxin may move through the interstitial fluids and the circulatory systems, such as, e.g., the cardiovascular system and the lymphatic system, of the body, resulting in the undesirable dispersal of the toxin to areas not targeted for toxin treatment. Such dispersal can lead to undesirable side effects, such as, e.g., inhibition of neurotransmitter release in neurons not targeted for treatment or paralysis of a muscle not targeted for treatment. For example, a patient administered a therapeutically effective amount of a BoNT/A treatment into the neck muscles for torticollis may develop dysphagia because of dispersal of the toxin into the oropharynx. As another example, a patient administered a therapeutically effective amount of a BoNT/A treatment into the bladder for overactive bladder may develop dry mouth and/or dry eyes. Thus, there remains a need for improved Clostridial toxins that are effective at the site of treatment, but have negligible to minimal effects in areas not targeted for a toxin treatment.

A Clostridial toxin treatment inhibits neurotransmitter release by disrupting the exocytotic process used to secret the neurotransmitter into the synaptic cleft. There is a great desire by the pharmaceutical industry to expand the use of Clostridial toxin therapies beyond its current myo-relaxant applications to treat other nerve-based ailments, such as, e.g., various kinds of chronic pain, neurogenic inflammation and urogentital disorders, as well as other disorders, such as, e.g., pancreatitis. One approach that is currently being exploited to expand Clostridial toxin-based therapies involves modifying a Clostridial toxin so that the modified toxin has an altered cell targeting capability for a non-Clostridial toxin target cell. This re-targeted capability is achieved by replacing a naturally-occurring targeting domain of a Clostridial toxin with a targeting domain showing a preferential binding activity for a non-Clostridial toxin receptor present in a non-Clostridial toxin target cell. Such modifications to a targeting domain result in a Clostridial toxin chimeric called a Targeted Vesicular Exocytosis Modulating Protein (TVEMP) that is able to selectively bind to a non-Clostridial toxin receptor (target receptor) present on a non-Clostridial toxin target cell (re-targeted). A Clostridial toxin chimeric with a targeting activity for a non-Clostridial toxin target cell can bind to a receptor present on the non-Clostridial toxin target cell, translocate into the cytoplasm, and exert its proteolytic effect on the SNARE complex of the non-Clostridial toxin target cell.

Neurogenic inflammation encompasses a series of vascular and non-vascular inflammatory responses mediated by a complex biological process that ultimately results in the local release of inflammatory mediators and sensitizing compounds from sensory neurons. Upon insult by a noxious stimulus, such as, e.g., a pathogen, damage to cells, or an irritant, inflammation mediating and sensitizing molecules, such as, e.g., histamine, prostaglandins, leukotrienes, serotonin, neutral proteases, cytokines, bradykinin and nitric oxide, are released from inflammation mediating cells, such as, e.g., mast cells, immune cells, vascular endothelial cells, and vascular smooth muscle cells. See Jennelle Durnett Richardson and Michael R. Vasko, Cellular Mechanisms of Neurogenic Inflammation, 302(3) J. Pharmacol. Exp. Ther. 839-845 (2002), which is hereby incorporated by reference in its entirety. These inflammation mediating and sensitizing molecules act on sensory neurons to stimulate the release of inflammation inducing molecules such as, e.g., neuropeptides like substance P (SP) and calcitonin gene-related peptide (CGRP), prostaglandins, and amino acids like glutamate, from the peripheral nerve endings. Upon release, these inflammation inducing molecules are responsible for eliciting an inflammatory response, typically characterized by edema (swelling secondary to plasma extravasation), hypersensitivity (secondary to alterations in the excitability of certain sensory neurons), and an erythema (redness and warmth secondary to vasodilation) which extends beyond the site of stimulation (the flare response). Id. Because the underlying inflammatory symptoms are triggered by the activation of primary sensory neurons and the subsequent release of inflammation inducing molecules, the response is termed neurogenic inflammation.

Normally, neurogenic inflammation serves as a protective mechanism by an organism to remove noxious stimuli as well as initiate the healing process for injured tissue. This acute neurogenic inflammation forms the first line of defense by maintaining tissue integrity and contributing to tissue repair. In fact, in the absence of acute neurogenic inflammation, wounds and infections would never heal and progressive destruction of the tissue would compromise the survival of the organism. However, severe or prolonged noxious stimulation results in a chronic neurogenic inflammatory response provoking injury rather than mediating repair. This chronic neurogenic inflammation has been implicated in the pathophysiology of a wide range of unrelated disorders which underly a wide variety of human diseases.

Attempts to treat chronic neurogenic inflammation have met with limited success. This is due, in part, to the fact that the etiology of chronic neurogenic inflammation is a complex response based in part on the various inflammation inducing molecules and the multitude of inflammation mediating and sensitizing molecules that appear to elicit inflammation via redundant mechanism. See Richardson & Vasko, 302(3) J. Pharmacol. Exp. Ther. 839-845 (2002). Therefore, compounds and methods that can prevent the chronic release of inflammation inducing molecules from sensory neurons would be highly desirable for the treatment of chronic neurogenic inflammation.

The present specification discloses TVEMP compositions and methods for treating an individual suffering from chronic neurogenic inflammation. This is accomplished by administering a therapeutically effective amount of a composition comprising a TVEMP to an individual in need thereof. The disclosed methods provide a safe, inexpensive, out patient-based treatment for the treatment of chronic neurogenic inflammation.

Thus, aspects of the present invention provide a composition comprising a TVEMP comprising a retargeted peptide binding domain, a Clostridial toxin translocation domain and a Clostridial toxin enzymatic domain. A composition comprising a TVEMP can be a pharmaceutical composition. Such a pharmaceutical composition can comprise, in addition to a TVEMP, a pharmaceutical carrier, a pharmaceutical component, or both.

Other aspects of the present invention provide a method of treating neurogenic inflammation in a mammal, the method comprising the step of administering to the mammal in need thereof a therapeutically effective amount of a composition including a TVEMP comprising a retargeted peptide binding domain, a Clostridial toxin translocation domain and a Clostridial toxin enzymatic domain, wherein administration of the composition reduces the release of an inflammation inducing molecule, thereby reducing a symptom associated with chronic neurogenic inflammation.

Other aspects of the present invention provide a method of treating neurogenic inflammation in a mammal, the method comprising the step of administering to the mammal in need thereof a therapeutically effective amount of a composition including a TVEMP comprising a retargeted peptide binding domain, a Clostridial toxin translocation domain, a Clostridial toxin enzymatic domain, and an exogenous protease cleavage site, wherein administration of the composition reduces the release of an inflammation inducing molecule, thereby reducing a symptom associated with chronic neurogenic inflammation.

Still other aspects of the present invention provide a manufacturing of a medicament for treating urogenital-neurological disorder in a mammal in need thereof, the medicament comprising a TVEMP including a retargeted peptide binding domain, a Clostridial toxin translocation domain and a Clostridial toxin enzymatic domain.

Still aspects of the present invention provide a use of a composition for treating chronic neurogenic inflammation in a mammal in need thereof, the use comprising the step of administering to the mammal in need thereof a therapeutically effective amount of the composition, wherein the composition comprises a TVEMP including a retargeted peptide binding domain, a Clostridial toxin translocation domain and a Clostridial toxin enzymatic domain and wherein administration of the composition reduces the release of an inflammation inducing molecule, thereby treating the mammal. Still aspects of the present invention provide a use of a composition for treating chronic neurogenic inflammation in a mammal in need thereof, the use comprising the step of administering to the mammal in need thereof a therapeutically effective amount of the composition, wherein the composition comprises a TVEMP including a retargeted peptide binding domain, a Clostridial toxin translocation domain and a Clostridial toxin enzymatic domain and wherein administration of the composition educes a symptom of the chronic neurogenic inflammation, thereby treating the mammal.

### BRIEF DESCRIPTION OF THE DRAWINGS

FIG. 1 shows a schematic of the current paradigm of neurotransmitter release and Clostridial toxin intoxication in a central and peripheral neuron. FIG. 1A shows a schematic for the neurotransmitter release mechanism of a central and peripheral neuron. The release process can be described as comprising two steps: 1) vesicle docking, where the vesicle-bound SNARE protein of a vesicle containing neurotransmitter molecules associates with the membrane-bound SNARE proteins located at the plasma membrane; and 2) neurotransmitter release, where the vesicle fuses with the plasma membrane and the neurotransmitter molecules are exocytosed. FIG. 1B shows a schematic of the intoxication mechanism for tetanus and botulinum toxin activity in a central and peripheral neuron. This intoxication process can be described as comprising four steps: 1) receptor binding, where a Clostridial toxin binds to a Clostridial receptor system and initiates the intoxication process; 2) complex internalization, where after toxin binding, a vesicle containing the toxin/receptor system complex is endocytosed into the cell; 3) light chain translocation, where multiple events are thought to occur, including, e.g., changes in the internal pH of the vesicle, formation of a channel pore comprising the translocation domain of the Clostridial toxin heavy chain, separation of the Clostridial toxin light chain from the heavy chain, and release of the active light chain and 4) enzymatic target modification, where the activate light chain of Clostridial toxin proteolytically cleaves its target SNARE substrate, such as, e.g., SNAP-25, VAMP or Syntaxin, thereby preventing vesicle docking and neurotransmitter release.

FIG. 2 shows the domain organization of naturally-occurring Clostridial toxins. The single-chain form depicts the amino to carboxyl linear organization comprising an enzymatic domain, a translocation domain, and a retargeted peptide binding domain. The di-chain loop region located between the translocation and enzymatic domains is depicted by the double SS bracket. This region comprises an endogenous di-chain loop protease cleavage site that upon proteolytic cleavage with a naturally-occurring protease, such as, e.g., an endogenous Clostridial toxin protease or a naturally-occurring protease produced in the environment, converts the single-chain form of the toxin into the di-chain form. Above the single-chain form, the HCC region of the Clostridial toxin binding domain is depicted. This region comprises the β-trefoil domain which comprises in an amino to carboxyl linear organization an α-fold, a β4/β5 hairpin turn, a β-fold, a β8/β9 hairpin turn and a γ-fold.

FIG. 3 shows TVEMPs with an enhanced targeting domain located at the amino terminus of the modified toxin. FIG. 3A depicts the single-chain polypeptide form of a TVEMP with an amino to carboxyl linear organization comprising a binding element, a translocation element, a di-chain loop region comprising an exogenous protease cleavage site (P), and a therapeutic element. Upon proteolytic cleavage with a P protease, the single-chain form of the toxin is converted to the di-chain form. FIG. 3B depicts the single polypeptide form of a TVEMP with an amino to carboxyl linear organization comprising a binding element, a therapeutic element, a di-chain loop region comprising an exogenous protease cleavage site (P), and a translocation element. Upon proteolytic cleavage with a P protease, the single-chain form of the toxin is converted to the di-chain form.

FIG. 4 shows TVEMPs with an enhanced targeting domain located between the other two domains. FIG. 4A depicts the single polypeptide form of a TVEMP with an amino to carboxyl linear organization comprising a therapeutic element, a di-chain loop region comprising an exogenous protease cleavage site (P), a binding element, and a translocation element. Upon proteolytic cleavage with a P protease, the single-chain form of the toxin is converted to the di-chain form. FIG. 4B depicts the single polypeptide form of a TVEMP with an amino to carboxyl linear organization comprising a translocation element, a di-chain loop region comprising an exogenous protease cleavage site (P), a binding element, and a therapeutic element. Upon proteolytic cleavage with a P protease, the single-chain form of the toxin is converted to the di-chain form. FIG. 4C depicts the single polypeptide form of a TVEMP with an amino to carboxyl linear organization comprising a therapeutic element, a binding element, a di-chain loop region comprising an exogenous protease cleavage site (P), and a translocation element. Upon proteolytic cleavage with a P protease, the single-chain form of the toxin is converted to the di-chain form. FIG. 4D depicts the single polypeptide form of a TVEMP with an amino to carboxyl linear organization comprising a translocation element, a binding element, a di-chain loop region comprising an exogenous protease cleavage site (P), and a therapeutic element. Upon proteolytic cleavage with a P protease, the single-chain form of the toxin is converted to the di-chain form.

FIG. 5 shows TVEMPs with an enhanced targeting domain located at the carboxyl terminus of the modified toxin. FIG. 5A depicts the single polypeptide form of a TVEMP with an amino to carboxyl linear organization comprising a therapeutic element, a di-chain loop region comprising an exogenous protease cleavage site (P), a translocation element, and a binding element. Upon proteolytic cleavage with a P protease, the single-chain form of the toxin is converted to the di-chain form. FIG. 5B depicts the single polypeptide form of a TVEMP with an amino to carboxyl linear organization comprising a translocation element, a di-chain loop region comprising an exogenous protease cleavage site (P), a therapeutic element, and a binding element. Upon proteolytic cleavage with a P protease, the single-chain form of the toxin is converted to the di-chain form.

### DETAILED DESCRIPTION

Aspects of the present invention provide, in part, a TVEMP. As used herein, a “Targeted Vesicular Exocytosis Modulating Protein” is synonomous with “TVEMP” and refers to any molecule comprising a retargeted peptide binding domain, a Clostridial toxin translocation domain and a Clostridial toxin enzymatic domain. Exemplary TVEMPs useful to practice aspects of the present invention are disclosed in, e.g., Steward, L. E. et al., Modified Clostridial Toxins with Enhanced Translocation Capabilities and Altered Targeting Activity For Non-Clostridial Toxin Target Cells, U.S. patent application Ser. No. 11/776,075 (Jul. 11, 2007); Dolly, J. O. et al., Activatable Clostridial Toxins, U.S. patent application Ser. No. 11/829,475 (Jul. 27, 2007); Foster, K. A. et al., Fusion Proteins, International Patent Publication WO 2006/059093 (Jun. 8, 2006); and Foster, K. A. et al., Non-Cytotoxic Protein Conjugates, International Patent Publication WO 2006/059105 (Jun. 8, 2006), each of which is incorporated by reference in its entirety.

Clostridial toxins produced by Clostridium botulinum, Clostridium tetani, Clostridium baratii and Clostridium butyricum are the most widely used in therapeutic and cosmetic treatments of humans and other mammals. Strains of C. botulinum produce seven antigenically-distinct types of Botulinum toxins (BoNTs), which have been identified by investigating botulism outbreaks in man (BoNT/A, /B, /E and /F), animals (BoNT/C1 and /D), or isolated from soil (BoNT/G). BoNTs possess approximately 35% amino acid identity with each other and share the same functional domain organization and overall structural architecture. It is recognized by those of skill in the art that within each type of Clostridial toxin there can be subtypes that differ somewhat in their amino acid sequence, and also in the nucleic acids encoding these proteins. For example, there are presently four BoNT/A subtypes, BoNT/A1, BoNT/A2, BoNT/A3 and BoNT/A4, with specific subtypes showing approximately 89% amino acid identity when compared to another BoNT/A subtype. While all seven BoNT serotypes have similar structure and pharmacological properties, each also displays heterogeneous bacteriological characteristics. In contrast, tetanus toxin (TeNT) is produced by a uniform group of C. tetani. Two other species of Clostridia, C. baratii and C. butyricum, also produce toxins, BaNT and BuNT respectively, which are similar to BoNT/F and BoNT/E, respectively.

Each mature di-chain molecule comprises three functionally distinct domains: 1) an enzymatic domain located in the light chain (LC) that includes a metalloprotease region containing a zinc-dependent endopeptidase activity which specifically targets core components of the neurotransmitter release apparatus; 2) a translocation domain (H_{N}) contained within the amino-terminal half of the heavy chain (HC) that facilitates release of the LC from intracellular vesicles into the cytoplasm of the target cell; and 3) a binding domain (H_{C}) found within the carboxyl-terminal half of the HC that determines the binding activity and binding specificity of the toxin to the receptor complex located at the surface of the target cell. The H_{C} domain comprises two distinct structural features of roughly equal size that indicate function and are designated the H_{CN} and H_{CC} subdomains. Table 1 gives approximate boundary regions for each domain found in exemplary Clostridial toxins.

TABLE 1
Clostridial Toxin Reference Sequences and Regions

| Toxin | SEQ ID NO: | LC | H_{N} | H_{C} |
|---|---|---|---|---|
| BoNT/A | 1 | M1-K448 | A449-K871 | N872-L1296 |
| BoNT/B | 2 | M1-K441 | A442-S858 | E859-E1291 |
| BoNT/C1 | 3 | M1-K449 | T450-N866 | N867-E1291 |
| BoNT/D | 4 | M1-R445 | D446-N862 | S863-E1276 |
| BoNT/E | 5 | M1-R422 | K423-K845 | R846-K1252 |
| BoNT/F | 6 | M1-K439 | A440-K864 | K865-E1274 |
| BoNT/G | 7 | M1-K446 | S447-S863 | N864-E1297 |
| TeNT | 8 | M1-A457 | S458-V879 | I880-D1315 |
| BaNT | 9 | M1-K431 | N432-I857 | I858-E1268 |
| BuNT | 10 | M1-R422 | K423-I847 | Y1086-K1251 |

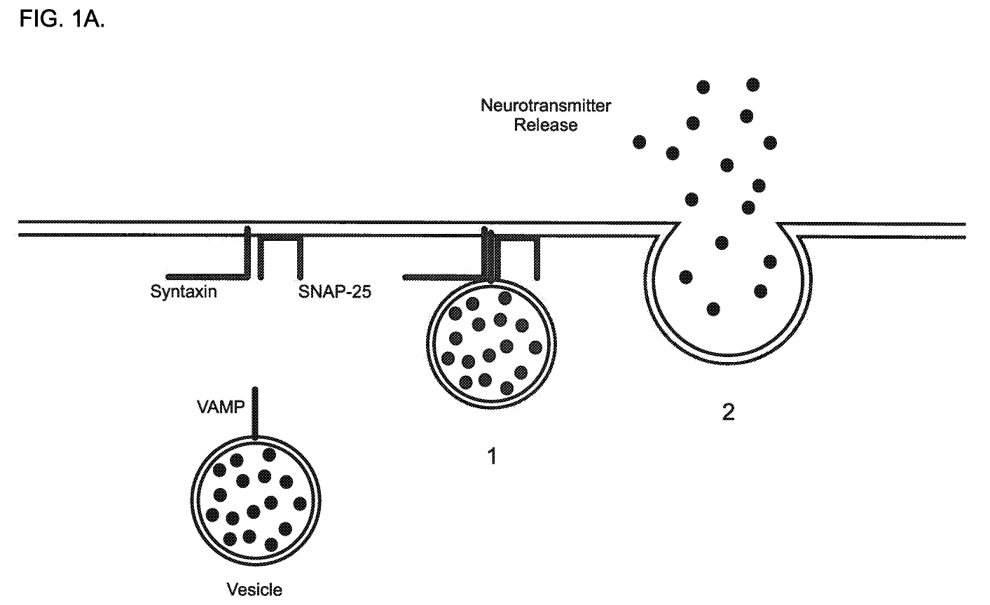

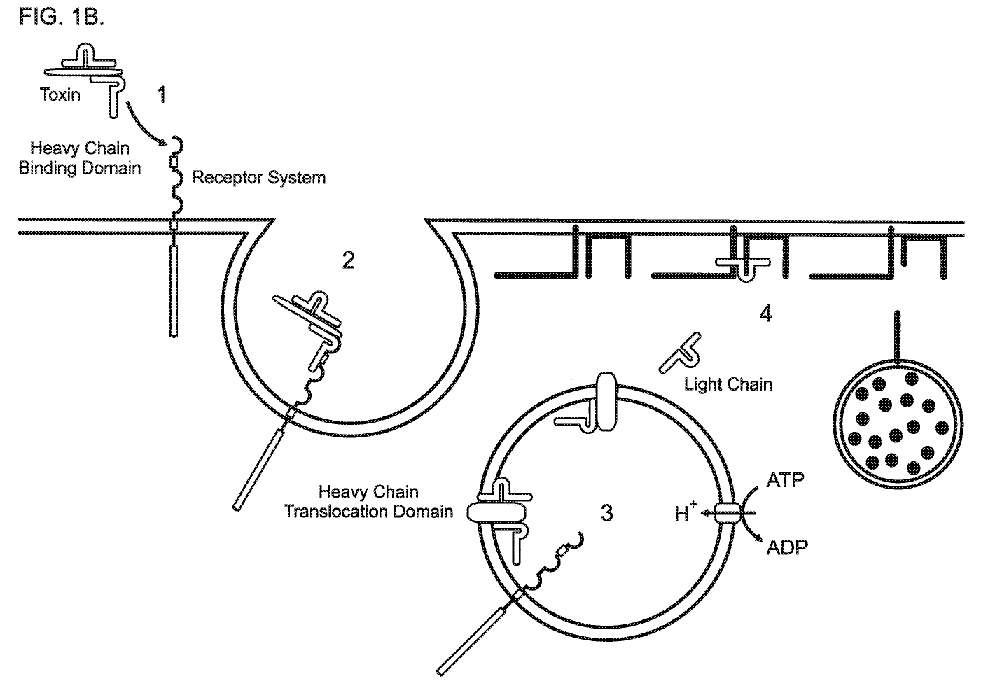

The binding, translocation and enzymatic activity of these three functional domains are all necessary for toxicity. While all details of this process are not yet precisely known, the overall cellular intoxication mechanism whereby Clostridial toxins enter a neuron and inhibit neurotransmitter release is similar, regardless of serotype or subtype. Although the applicants have no wish to be limited by the following description, the intoxication mechanism can be described as comprising at least four steps: 1) receptor binding, 2) complex internalization, 3) light chain translocation, and 4) enzymatic target modification (see FIG. 1). The process is initiated when the H_{C} domain of a Clostridial toxin binds to a toxin-specific receptor system located on the plasma membrane surface of a target cell. The binding specificity of a receptor complex is thought to be achieved, in part, by specific combinations of gangliosides and protein receptors that appear to distinctly comprise each Clostridial toxin receptor complex. Once bound, the toxin/receptor complexes are internalized by endocytosis and the internalized vesicles are sorted to specific intracellular routes. The translocation step appears to be triggered by the acidification of the vesicle compartment. This process seems to initiate two important pH-dependent structural rearrangements that increase hydrophobicity and promote formation di-chain form of the toxin. Once activated, light chain endopeptidase of the toxin is released from the intracellular vesicle into the cytosol where it appears to specifically target one of three known core components of the neurotransmitter release apparatus. These core proteins, vesicle-associated membrane protein (VAMP)/synaptobrevin, synaptosomal-associated protein of 25 kDa (SNAP-25) and Syntaxin, are necessary for synaptic vesicle docking and fusion at the nerve terminal and constitute members of the soluble N-ethylmaleimide-sensitive factor-attachment protein-receptor (SNARE) family. BoNT/A and BoNT/E cleave SNAP-25 in the carboxyl-terminal region, releasing a nine or twenty-six amino acid segment, respectively, and BoNT/C1 also cleaves SNAP-25 near the carboxyl-terminus. The botulinum serotypes BoNT/B, BoNT/D, BoNT/F and BoNT/G, and tetanus toxin, act on the conserved central portion of VAMP, and release the amino-terminal portion of VAMP into the cytosol. BoNT/C1 cleaves syntaxin at a single site near the cytosolic membrane surface. The selective proteolysis of synaptic SNAREs accounts for the block of neurotransmitter release caused by Clostridial toxins in vivo. The SNARE protein targets of Clostridial toxins are common to exocytosis in a variety of non-neuronal types; in these cells, as in neurons, light chain peptidase activity inhibits exocytosis, see, e.g., Yann Humeau et al., How Botulinum and Tetanus Neurotoxins Block Neurotransmitter Release, 82(5) Biochimie. 427-446 (2000); Kathryn Turton et al., Botulinum and Tetanus Neurotoxins: Structure, Function and Therapeutic Utility, 27(11) Trends Biochem. Sci. 552-558. (2002); Giovanna Lalli et al., The Journey of Tetanus and Botulinum Neurotoxins in Neurons, 11(9) Trends Microbiol. 431-437, (2003).

In an aspect of the invention, a TVEMP comprises, in part, a Clostridial toxin enzymatic domain. As used herein, the term “Clostridial toxin enzymatic domain” refers to any Clostridial toxin polypeptide that can execute the enzymatic target modification step of the intoxication process. Thus, a Clostridial toxin enzymatic domain specifically targets a Clostridial toxin substrate and encompasses the proteolytic cleavage of a Clostridial toxin substrate, such as, e.g., SNARE proteins like a SNAP-25 substrate, a VAMP substrate and a Syntaxin substrate. Non-limiting examples of a Clostridial toxin enzymatic domain include, e.g., a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, and a BuNT enzymatic domain. Other non-limiting examples of a Clostridial toxin enzymatic domain include, e.g., amino acids 1-448 of SEQ ID NO: 1, amino acids 1-441 of SEQ ID NO: 2, amino acids 1-449 of SEQ ID NO: 3, amino acids 1-445 of SEQ ID NO: 4, amino acids 1-422 of SEQ ID NO: 5, amino acids 1-439 of SEQ ID NO: 6, amino acids 1-446 of SEQ ID NO: 7, amino acids 1-457 of SEQ ID NO: 8, amino acids 1-431 of SEQ ID NO: 9, and amino acids 1-422 of SEQ ID NO: 10.

A Clostridial toxin enzymatic domain includes, without limitation, naturally occurring Clostridial toxin enzymatic domain variants, such as, e.g., Clostridial toxin enzymatic domain isoforms and Clostridial toxin enzymatic domain subtypes; and non-naturally occurring Clostridial toxin enzymatic domain variants, such as, e.g., conservative Clostridial toxin enzymatic domain variants, non-conservative Clostridial toxin enzymatic domain variants, Clostridial toxin enzymatic domain chimerics, active Clostridial toxin enzymatic domain fragments thereof, or any combination thereof.

As used herein, the term “Clostridial toxin enzymatic domain variant,” whether naturally-occurring or non-naturally-occurring, refers to a Clostridial toxin enzymatic domain that has at least one amino acid change from the corresponding region of the disclosed reference sequences (Table 1) and can be described in percent identity to the corresponding region of that reference sequence. Unless expressly indicated, Clostridial toxin enzymatic domain variants useful to practice disclosed embodiments are variants that execute the enzymatic target modification step of the intoxication process. As non-limiting examples, a BoNT/A enzymatic domain variant comprising amino acids 1-448 of SEQ ID NO: 1 will have at least one amino acid difference, such as, e.g., an amino acid substitution, deletion or addition, as compared to the amino acid region 1-448 of SEQ ID NO: 1; a BoNT/B enzymatic domain variant comprising amino acids 1-441 of SEQ ID NO: 2 will have at least one amino acid difference, such as, e.g., an amino acid substitution, deletion or addition, as compared to the amino acid region 1-441 of SEQ ID NO: 2; a BoNT/C1 enzymatic domain variant comprising amino acids 1-449 of SEQ ID NO: 3 will have at least one amino acid difference, such as, e.g., an amino acid substitution, deletion or addition, as compared to the amino acid region 1-449 of SEQ ID NO: 3; a BoNT/D enzymatic domain variant comprising amino acids 1-445 of SEQ ID NO: 4 will have at least one amino acid difference, such as, e.g., an amino acid substitution, deletion or addition, as compared to the amino acid region 1-445 of SEQ ID NO: 4; a BoNT/E enzymatic domain variant comprising amino acids 1-422 of SEQ ID NO: 5 will have at least one amino acid difference, such as, e.g., an amino acid substitution, deletion or addition, as compared to the amino acid region 1-422 of SEQ ID NO: 5; a BoNT/F enzymatic domain variant comprising amino acids 1-439 of SEQ ID NO: 6 will have at least one amino acid difference, such as, e.g., an amino acid substitution, deletion or addition, as compared to the amino acid region 1-439 of SEQ ID NO: 6; a BoNT/G enzymatic domain variant comprising amino acids 1-446 of SEQ ID NO: 7 will have at least one amino acid difference, such as, e.g., an amino acid substitution, deletion or addition, as compared to the amino acid region 1-446 of SEQ ID NO: 7; and a TeNT enzymatic domain variant comprising amino acids 1-457 of SEQ ID NO: 8 will have at least one amino acid difference, such as, e.g., an amino acid substitution, deletion or addition, as compared to the amino acid region 1-457 of SEQ ID NO: 8.

It is recognized by those of skill in the art that within each serotype of Clostridial toxin there can be naturally occurring Clostridial toxin enzymatic domain variants that differ somewhat in their amino acid sequence, and also in the nucleic acids encoding these proteins. For example, there are presently five BoNT/A subtypes, BoNT/A1, BoNT/A2, BoNT/A3, BoNT/A4, and BoNT/A5, with specific enzymatic domain subtypes showing approximately 95% amino acid identity when compared to another BoNT/A enzymatic domain subtype. As used herein, the term “naturally occurring Clostridial toxin enzymatic domain variant” refers to any Clostridial toxin enzymatic domain produced by a naturally-occurring process, including, without limitation, Clostridial toxin enzymatic domain isoforms produced from alternatively-spliced transcripts, Clostridial toxin enzymatic domain isoforms produced by spontaneous mutation and Clostridial toxin enzymatic domain subtypes. A naturally occurring Clostridial toxin enzymatic domain variant can function in substantially the same manner as the reference Clostridial toxin enzymatic domain on which the naturally occurring Clostridial toxin enzymatic domain variant is based, and can be substituted for the reference Clostridial toxin enzymatic domain in any aspect of the present invention.

A non-limiting example of a naturally occurring Clostridial toxin enzymatic domain variant is a Clostridial toxin enzymatic domain isoform such as, e.g., a BoNT/A enzymatic domain isoform, a BoNT/B enzymatic domain isoform, a BoNT/C1 enzymatic domain isoform, a BoNT/D enzymatic domain isoform, a BoNT/E enzymatic domain isoform, a BoNT/F enzymatic domain isoform, a BoNT/G enzymatic domain isoform, and a TeNT enzymatic domain isoform. Another non-limiting example of a naturally occurring Clostridial toxin enzymatic domain variant is a Clostridial toxin enzymatic domain subtype such as, e.g., an enzymatic domain from subtype BoNT/A1, BoNT/A2, BoNT/A3, BoNT/A4 and BoNT/A5; an enzymatic domain from subtype BoNT/B1, BoNT/B2, BoNT/B bivalent and BoNT/B nonproteolytic; an enzymatic domain from subtype BoNT/C1-1 and BoNT/C1-2; an enzymatic domain from subtype BoNT/E1, BoNT/E2 and BoNT/E3; and an enzymatic domain from subtype BoNT/F1, BoNT/F2, BoNT/F3 and BoNT/F4.

As used herein, the term “non-naturally occurring Clostridial toxin enzymatic domain variant” refers to any Clostridial toxin enzymatic domain produced with the aid of human manipulation, including, without limitation, Clostridial toxin enzymatic domains produced by genetic engineering using random mutagenesis or rational design and Clostridial toxin enzymatic domains produced by chemical synthesis. Non-limiting examples of non-naturally occurring Clostridial toxin enzymatic domain variants include, e.g., conservative Clostridial toxin enzymatic domain variants, non-conservative Clostridial toxin enzymatic domain variants, Clostridial toxin enzymatic domain chimeric variants and active Clostridial toxin enzymatic domain fragments.

As used herein, the term “conservative Clostridial toxin enzymatic domain variant” refers to a Clostridial toxin enzymatic domain that has at least one amino acid substituted by another amino acid or an amino acid analog that has at least one property similar to that of the original amino acid from the reference Clostridial toxin enzymatic domain sequence (Table 1). Examples of properties include, without limitation, similar size, topography, charge, hydrophobicity, hydrophilicity, lipophilicity, covalent-bonding capacity, hydrogen-bonding capacity, a physicochemical property, of the like, or any combination thereof. A conservative Clostridial toxin enzymatic domain variant can function in substantially the same manner as the reference Clostridial toxin enzymatic domain on which the conservative Clostridial toxin enzymatic domain variant is based, and can be substituted for the reference Clostridial toxin enzymatic domain in any aspect of the present invention. Non-limiting examples of a conservative Clostridial toxin enzymatic domain variant include, e.g., conservative BoNT/A enzymatic domain variants, conservative BoNT/B enzymatic domain variants, conservative BoNT/C1 enzymatic domain variants, conservative BoNT/D enzymatic domain variants, conservative BoNT/E enzymatic domain variants, conservative BoNT/F enzymatic domain variants, conservative BoNT/G enzymatic domain variants, and conservative TeNT enzymatic domain variants.

As used herein, the term “non-conservative Clostridial toxin enzymatic domain variant” refers to a Clostridial toxin enzymatic domain in which 1) at least one amino acid is deleted from the reference Clostridial toxin enzymatic domain on which the non-conservative Clostridial toxin enzymatic domain variant is based; 2) at least one amino acid added to the reference Clostridial toxin enzymatic domain on which the non-conservative Clostridial toxin enzymatic domain is based; or 3) at least one amino acid is substituted by another amino acid or an amino acid analog that does not share any property similar to that of the original amino acid from the reference Clostridial toxin enzymatic domain sequence (Table 1). A non-conservative Clostridial toxin enzymatic domain variant can function in substantially the same manner as the reference Clostridial toxin enzymatic domain on which the non-conservative Clostridial toxin enzymatic domain variant is based, and can be substituted for the reference Clostridial toxin enzymatic domain in any aspect of the present invention. Non-limiting examples of a non-conservative Clostridial toxin enzymatic domain variant include, e.g., non-conservative BoNT/A enzymatic domain variants, non-conservative BoNT/B enzymatic domain variants, non-conservative BoNT/C1 enzymatic domain variants, non-conservative BoNT/D enzymatic domain variants, non-conservative BoNT/E enzymatic domain variants, non-conservative BoNT/F enzymatic domain variants, non-conservative BoNT/G enzymatic domain variants, and non-conservative TeNT enzymatic domain variants.

As used herein, the term “Clostridial toxin enzymatic domain chimeric” refers to a polypeptide comprising at least a portion of a Clostridial toxin enzymatic domain and at least a portion of at least one other polypeptide to form a toxin enzymatic domain with at least one property different from the reference Clostridial toxin enzymatic domains of Table 1, with the proviso that this Clostridial toxin enzymatic domain chimeric is still capable of specifically targeting the core components of the neurotransmitter release apparatus and thus participate in executing the overall cellular mechanism whereby a Clostridial toxin proteolytically cleaves a substrate. Such Clostridial toxin enzymatic domain chimerics are described in, e.g., Lance E. Steward et al., Leucine-based Motif and Clostridial Toxins, U.S. Patent Publication 2003/0027752 (Feb. 6, 2003); Lance E. Steward et al., Clostridial Neurotoxin Compositions and Modified Clostridial Neurotoxins, U.S. Patent Publication 2003/0219462 (Nov. 27, 2003); and Lance E. Steward et al., Clostridial Neurotoxin Compositions and Modified Clostridial Neurotoxins, U.S. Patent Publication 2004/0220386 (Nov. 4, 2004), each of which is incorporated by reference in its entirety.

As used herein, the term “active Clostridial toxin enzymatic domain fragment” refers to any of a variety of Clostridial toxin fragments comprising the enzymatic domain can be useful in aspects of the present invention with the proviso that these enzymatic domain fragments can specifically target the core components of the neurotransmitter release apparatus and thus participate in executing the overall cellular mechanism whereby a Clostridial toxin proteolytically cleaves a substrate. The enzymatic domains of Clostridial toxins are approximately 420-460 amino acids in length and comprise an enzymatic domain (Table 1). Research has shown that the entire length of a Clostridial toxin enzymatic domain is not necessary for the enzymatic activity of the enzymatic domain. As a non-limiting example, the first eight amino acids of the BoNT/A enzymatic domain (residues 1-8 of SEQ ID NO: 1) are not required for enzymatic activity. As another non-limiting example, the first eight amino acids of the TeNT enzymatic domain (residues 1-8 of SEQ ID NO: 8) are not required for enzymatic activity. Likewise, the carboxyl-terminus of the enzymatic domain is not necessary for activity. As a non-limiting example, the last 32 amino acids of the BoNT/A enzymatic domain (residues 417-448 of SEQ ID NO: 1) are not required for enzymatic activity. As another non-limiting example, the last 31 amino acids of the TeNT enzymatic domain (residues 427-457 of SEQ ID NO: 8) are not required for enzymatic activity. Thus, aspects of this embodiment can include Clostridial toxin enzymatic domains comprising an enzymatic domain having a length of, e.g., at least 350 amino acids, at least 375 amino acids, at least 400 amino acids, at least 425 amino acids and at least 450 amino acids. Other aspects of this embodiment can include Clostridial toxin enzymatic domains comprising an enzymatic domain having a length of, e.g., at most 350 amino acids, at most 375 amino acids, at most 400 amino acids, at most 425 amino acids and at most 450 amino acids.

Any of a variety of sequence alignment methods can be used to determine percent identity of naturally-occurring Clostridial toxin enzymatic domain variants and non-naturally-occurring Clostridial toxin enzymatic domain variants, including, without limitation, global methods, local methods and hybrid methods, such as, e.g., segment approach methods. Protocols to determine percent identity are routine procedures within the scope of one skilled in the art and from the teaching herein.

Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein Sequence Alignments by Iterative Refinement as Assessed by Reference to Structural Alignments, 264(4) J. Mol. Biol. 823-838 (1996).

Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics, 1428-1435 (2004).

Hybrid methods combine functional aspects of both global and local alignment methods. Non-limiting methods include, e.g., segment-to-segment comparison, see, e.g., Burkhard Morgenstern et al., Multiple DNA and Protein Sequence Alignment Based On Segment-To-Segment Comparison, 93(22) Proc. Natl. Acad. Sci. U.S.A. 12098-12103 (1996); T-Coffee, see, e.g., Cédric Notredame et al., T-Coffee: A Novel Algorithm for Multiple Sequence Alignment, 302(1) J. Mol. Biol. 205-217 (2000); MUSCLE, see, e.g., Robert C. Edgar, MUSCLE: Multiple Sequence Alignment With High Score Accuracy and High Throughput, 32(5) Nucleic Acids Res. 1792-1797 (2004); and DIALIGN-T, see, e.g., Amarendran R Subramanian et al., DIALIGN-T: An Improved Algorithm for Segment-Based Multiple Sequence Alignment, 6(1) BMC Bioinformatics 66 (2005).

The present specification describes various polypeptide variants where one amino acid is substituted for another, such as, e.g., Clostridial toxin variants, Clostridial toxin enzymatic domain variants, Clostridial toxin translocation domain variants, Clostridial toxin binding domain variants, non-Clostridial toxin binding domain variants, retargeted peptide binding domain variants, and protease cleavage site variants. A substitution can be assessed by a variety of factors, such as, e.g., the physic properties of the amino acid being substituted (Table 2) or how the original amino acid would tolerate a substitution (Table 3). The selections of which amino acid can be substituted for another amino acid in a polypeptide are known to a person of ordinary skill in the art.

TABLE 2
Amino Acid Properties

| Property | Amino Acids |
|---|---|
| Aliphatic | G, A, I, L, M, P, V |
| Aromatic | F, H, W, Y |
| C-beta branched | I, V, T |
| Hydrophobic | C, F, I, L, M, V, W |
| Small polar | D, N, P |
| Small non-polar | A, C, G, S, T |
| Large polar | E, H, K, Q, R, W, Y |
| Large non-polar | F, I, L, M, V |
| Charged | D, E, H, K, R |
| Uncharged | C, S, T |
| Negative | D, E |
| Positive | H, K, R |
| Acidic | D, E |
| Basic | K, R |
| Amide | N, Q |

TABLE 3
Amino Acid Substitutions

| | Favored | | |
|---|---|---|---|
| Amino Acid | Substitution | Neutral Substitutions | Disfavored substitution |
| A | G, S, T | C, E, I, K, M, L, P, Q, R, V | D, F, H, N, Y, W |
| C | F, S, Y, W | A, H, I, M, L, T, V | D, E, G, K, N, P, Q, R |
| D | E, N | G, H, K, P, Q, R, S, T | A, C, I, L, |
| E | D, K, Q | A, H, N, P, R, S, T | C, F, G, I, L, M, V, W, Y |
| F | M, L, W, Y | C, I, V | A, D, E, G, H, K, N, P, Q, R, S, T |
| G | A, S | D, K, N, P, Q, R | C, E, F, H, I, L, M, T, V, W, Y |
| H | N, Y | C, D, E, K, Q, R, S, T, W | A, F, G, I, L, M, P, V |
| I | V, L, M | A, C, T, F, Y | D, E, G, H, K, N, P, Q, R, S, W |
| K | Q, E, R | A, D, G, H, M, N, P, S, T | C, F, I, L, V, W, Y |
| L | F, I, M, V | A, C, W, Y | D, E, G, H, K, N, P, Q, R, S, T |
| M | F, I, L, V | A, C, R, Q, K, T, W, Y | D, E, G, H, N, P, S |
| N | D, H, S | E, G, K, Q, R, T | A, C, F, I, L, M, P, V, W, Y |
| P | — | A, D, E, G, K, Q, R, S, T | C, F, H, I, L, M, N, V, W, Y |
| Q | E, K, R | A, D, G, H, M, N, P, S, T | C, F, I, L, V, W, Y |
| R | K, Q | A, D, E, G, H, M, N, P, S, T | C, F, I, L, V, W, Y |
| S | A, N, T | C, D, E, G, H, K, P, Q, R, T | F, I, L, M, V, W, Y |
| T | S | A, C, D, E, H, I, K, M, N, | |
| | | P, Q, R, V | F, G, L, W, Y |
| V | I, L, M | A, C, F, T, Y | D, E, G, H, K, N, P, Q, R, S, W |
| W | F, Y | H, L, M | A, C, D, E, G, I, K, N, P, Q, R, S, |
| | | | T, V |
| Y | F, H, W | C, I, L, M, V | A, D, E, G, K, N, P, Q, R, S, T |

Matthew J. Betts and Robert, B. Russell, Amino Acid Properties and Consequences of Substitutions, pp. 289-316, In Bioinformatics for Geneticists, (eds Michael R. Barnes, Ian C. Gray, Wiley, 2003).

Thus, in an embodiment, a TVEMP disclosed in the present specification comprises a Clostridial toxin enzymatic domain. In an aspect of this embodiment, a Clostridial toxin enzymatic domain comprises a naturally occurring Clostridial toxin enzymatic domain variant, such as, e.g., a Clostridial toxin enzymatic domain isoform or a Clostridial toxin enzymatic domain subtype. In another aspect of this embodiment, a Clostridial toxin enzymatic domain comprises a non-naturally occurring Clostridial toxin enzymatic domain variant, such as, e.g., a conservative Clostridial toxin enzymatic domain variant, a non-conservative Clostridial toxin enzymatic domain variant, a Clostridial toxin chimeric enzymatic domain, an active Clostridial toxin enzymatic domain fragment, or any combination thereof.

In another embodiment, a Clostridial toxin enzymatic domain comprises a BoNT/A enzymatic domain. In an aspect of this embodiment, a BoNT/A enzymatic domain comprises amino acids 1-448 of SEQ ID NO: 1. In another aspect of this embodiment, a BoNT/A enzymatic domain comprises a naturally occurring BoNT/A enzymatic domain variant, such as, e.g., an enzymatic domain from a BoNT/A isoform or an enzymatic domain from a BoNT/A subtype. In another aspect of this embodiment, a BoNT/A enzymatic domain comprises amino acids 1-448 of a naturally occurring BoNT/A enzymatic domain variant of SEQ ID NO: 1, such as, e.g., amino acids 1-448 of a BoNT/A isoform of SEQ ID NO: 1 or amino acids 1-448 of a BoNT/A subtype of SEQ ID NO: 1. In still another aspect of this embodiment, a BoNT/A enzymatic domain comprises a non-naturally occurring BoNT/A enzymatic domain variant, such as, e.g., a conservative BoNT/A enzymatic domain variant, a non-conservative BoNT/A enzymatic domain variant, a BoNT/A chimeric enzymatic domain, an active BoNT/A enzymatic domain fragment, or any combination thereof. In still another aspect of this embodiment, a BoNT/A enzymatic domain comprises amino acids 1-448 of a non-naturally occurring BoNT/A enzymatic domain variant of SEQ ID NO: 1, such as, e.g., amino acids 1-448 of a conservative BoNT/A enzymatic domain variant of SEQ ID NO: 1, amino acids 1-448 of a non-conservative BoNT/A enzymatic domain variant of SEQ ID NO: 1, amino acids 1-448 of an active BoNT/A enzymatic domain fragment of SEQ ID NO: 1, or any combination thereof.

In other aspects of this embodiment, a BoNT/A enzymatic domain comprises a polypeptide having an amino acid identity of, e.g., at least 70%, at least 75%, at least 80%, at least 85%, at least 90%, at least 95%, or at least 97% to amino acids 1-448 of SEQ ID NO: 1; or at most 70%, at most 75%, at most 80%, at most 85%, at most 90%, at most 95%, or at most 97% to amino acids 1-448 of SEQ ID NO: 1. In yet other aspects of this embodiment, a BoNT/A enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 1-448 of SEQ ID NO: 1; or at most 1, 2, 3, 4, 5, 6, 7, 8, 9, 10, 20, 30, 40, 50, or 100 non-contiguous amino acid deletions, additions, and/or substitutions relative to amino acids 1-448 of SEQ ID NO: 1. In still other aspects of this embodiment, a BoNT/A enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 1-448 of SEQ ID NO: 1; or at most 1, 2, 3, 4, 5, 6, 7, 8, 9, 10, 20, 30, 40, 50, or 100 contiguous amino acid deletions, additions, and/or substitutions relative to amino acids 1-448 of SEQ ID NO: 1.

In another embodiment, a Clostridial toxin enzymatic domain comprises a BoNT/B enzymatic domain. In an aspect of this embodiment, a BoNT/B enzymatic domain comprises amino acids 1-441 of SEQ ID NO: 2. In another aspect of this embodiment, a BoNT/B enzymatic domain comprises a naturally occurring BoNT/B enzymatic domain variant, such as, e.g., an enzymatic domain from a BoNT/B isoform or an enzymatic domain from a BoNT/B subtype. In another aspect of this embodiment, a BoNT/B enzymatic domain comprises amino acids 1-441 of a naturally occurring BoNT/B enzymatic domain variant of SEQ ID NO: 2, such as, e.g., amino acids 1-441 of a BoNT/B isoform of SEQ ID NO: 2 or amino acids 1-441 of a BoNT/B subtype of SEQ ID NO: 2. In still another aspect of this embodiment, a BoNT/B enzymatic domain comprises a non-naturally occurring BoNT/B enzymatic domain variant, such as, e.g., a conservative BoNT/B enzymatic domain variant, a non-conservative BoNT/B enzymatic domain variant, a BoNT/B chimeric enzymatic domain, an active BoNT/B enzymatic domain fragment, or any combination thereof. In still another aspect of this embodiment, a BoNT/B enzymatic domain comprises amino acids 1-441 of a non-naturally occurring BoNT/B enzymatic domain variant of SEQ ID NO: 2, such as, e.g., amino acids 1-441 of a conservative BoNT/B enzymatic domain variant of SEQ ID NO: 2, amino acids 1-441 of a non-conservative BoNT/B enzymatic domain variant of SEQ ID NO: 2, amino acids 1-441 of an active BoNT/B enzymatic domain fragment of SEQ ID NO: 2, or any combination thereof.

In other aspects of this embodiment, a BoNT/B enzymatic domain comprises a polypeptide having an amino acid identity of, e.g., at least 70%, at least 75%, at least 80%, at least 85%, at least 90%, at least 95%, or at least 97% to amino acids 1-441 of SEQ ID NO: 2; or at most 70%, at most 75%, at most 80%, at most 85%, at most 90%, at most 95%, or at most 97% to amino acids 1-441 of SEQ ID NO: 2. In yet other aspects of this embodiment, a BoNT/B enzymatic domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 1-441 of SEQ ID NO: 2; or at most 1, 2, 3, 4, 5, 6, 7, 8, 9, 10, 20, 30, 40, 50, or 100 non-contiguous amino acid deletions, additions, and/or substitutions relative to amino acids 1-441 of SEQ ID NO: 2. In still other aspects of this embodiment, a BoNT/B enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 1-441 of SEQ ID NO: 2; or at most 1, 2, 3, 4, 5, 6, 7, 8, 9, 10, 20, 30, 40, 50, or 100 contiguous amino acid deletions, additions, and/or substitutions relative to amino acids 1-441 of SEQ ID NO: 2.

In another embodiment, a Clostridial toxin enzymatic domain comprises a BoNT/C1 enzymatic domain. In an aspect of this embodiment, a BoNT/C1 enzymatic domain comprises amino acids 1-449 of SEQ ID NO: 3. In another aspect of this embodiment, a BoNT/C1 enzymatic domain comprises a naturally occurring BoNT/C1 enzymatic domain variant, such as, e.g., an enzymatic domain from a BoNT/C1 isoform or an enzymatic domain from a BoNT/C1 subtype. In another aspect of this embodiment, a BoNT/C1 enzymatic domain comprises amino acids 1-449 of a naturally occurring BoNT/C1 enzymatic domain variant of SEQ ID NO: 3, such as, e.g., amino acids 1-449 of a BoNT/C1 isoform of SEQ ID NO: 3 or amino acids 1-449 of a BoNT/C1 subtype of SEQ ID NO: 3. In still another aspect of this embodiment, a BoNT/C1 enzymatic domain comprises a non-naturally occurring BoNT/C1 enzymatic domain variant, such as, e.g., a conservative BoNT/C1 enzymatic domain variant, a non-conservative BoNT/C1 enzymatic domain variant, a BoNT/C1 chimeric enzymatic domain, an active BoNT/C1 enzymatic domain fragment, or any combination thereof. In still another aspect of this embodiment, a BoNT/C1 enzymatic domain comprises amino acids 1-449 of a non-naturally occurring BoNT/C1 enzymatic domain variant of SEQ ID NO: 3, such as, e.g., amino acids 1-449 of a conservative BoNT/C1 enzymatic domain variant of SEQ ID NO: 3, amino acids 1-449 of a non-conservative BoNT/C1 enzymatic domain variant of SEQ ID NO: 3, amino acids 1-449 of an active BoNT/C1 enzymatic domain fragment of SEQ ID NO: 3, or any combination thereof.

In other aspects of this embodiment, a BoNT/C1 enzymatic domain comprises a polypeptide having an amino acid identity of, e.g., at least 70%, at least 75%, at least 80%, at least 85%, at least 90%, at least 95%, or at least 97% to amino acids 1-449 of SEQ ID NO: 3; or at most 70%, at most 75%, at most 80%, at most 85%, at most 90%, at most 95%, or at most 97% to amino acids 1-449 of SEQ ID NO: 3. In yet other aspects of this embodiment, a BoNT/C1 enzymatic domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 1-449 of SEQ ID NO: 3; or at most 1, 2, 3, 4, 5, 6, 7, 8, 9, 10, 20, 30, 40, 50, or 100 non-contiguous amino acid deletions, additions, and/or substitutions relative to amino acids 1-449 of SEQ ID NO: 3. In other aspects of this embodiment, a BoNT/C1 enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 1-449 of SEQ ID NO: 3; or at most 1, 2, 3, 4, 5, 6, 7, 8, 9, 10, 20, 30, 40, 50, or 100 contiguous amino acid deletions, additions, and/or substitutions relative to amino acids 1-449 of SEQ ID NO: 3.

In another embodiment, a Clostridial toxin enzymatic domain comprises a BoNT/D enzymatic domain. In an aspect of this embodiment, a BoNT/D enzymatic domain comprises amino acids 1-445 of SEQ ID NO: 4. In another aspect of this embodiment, a BoNT/D enzymatic domain comprises a naturally occurring BoNT/D enzymatic domain variant, such as, e.g., an enzymatic domain from a BoNT/D isoform or an enzymatic domain from a BoNT/D subtype. In another aspect of this embodiment, a BoNT/D enzymatic domain comprises amino acids 1-445 of a naturally occurring BoNT/D enzymatic domain variant of SEQ ID NO: 4, such as, e.g., amino acids 1-445 of a BoNT/D isoform of SEQ ID NO: 4 or amino acids 1-445 of a BoNT/D subtype of SEQ ID NO: 4. In still another aspect of this embodiment, a BoNT/D enzymatic domain comprises a non-naturally occurring BoNT/D enzymatic domain variant, such as, e.g., a conservative BoNT/D enzymatic domain variant, a non-conservative BoNT/D enzymatic domain variant, a BoNT/D chimeric enzymatic domain, an active BoNT/D enzymatic domain fragment, or any combination thereof. In still another aspect of this embodiment, a BoNT/D enzymatic domain comprises amino acids 1-445 of a non-naturally occurring BoNT/D enzymatic domain variant of SEQ ID NO: 4, such as, e.g., amino acids 1-445 of a conservative BoNT/D enzymatic domain variant of SEQ ID NO: 4, amino acids 1-445 of a non-conservative BoNT/D enzymatic domain variant of SEQ ID NO: 4, amino acids 1-445 of an active BoNT/D enzymatic domain fragment of SEQ ID NO: 4, or any combination thereof.

In other aspects of this embodiment, a BoNT/D enzymatic domain comprises a polypeptide having an amino acid identity of, e.g., at least 70%, at least 75%, at least 80%, at least 85%, at least 90%, at least 95%, or at least 97% to amino acids 1-445 of SEQ ID NO: 4; or at most 70%, at most 75%, at most 80%, at most 85%, at most 90%, at most 95%, or at most 97% to amino acids 1-445 of SEQ ID NO: 4. In yet other aspects of this embodiment, a BoNT/D enzymatic domain comprises a polypeptide having, e.g., at least 1, 2, 3, 4, 5, 6, 7, 8, 9, 10, 20, 30, 40, 50, or 100 non-contiguous amino acid deletions, additions and/or substitutions relative to amino acids 1-445 of SEQ ID NO: 4; or at most 1, 2, 3, 4, 5, 6, 7, 8, 9, 10, 20, 30, 40, 50, or 100 non-contiguous amino acid substitutions relative to amino acids 1-445 of SEQ ID NO: 4. In still other aspects of this embodiment, a BoNT/D enzymatic domain comprises a polypeptide having, e.g., at least 1, 2, 3, 4, 5, 6, 7, 8, 9, 10, 20, 30, 40, 50, or 100 contiguous amino acid deletions, additions and/or substitutions relative to amino acids 1-445 of SEQ ID NO: 4; or at most 1, 2, 3, 4, 5, 6, 7, 8, 9, 10, 20, 30, 40, 50, or 100 contiguous amino acid substitutions relative to amino acids 1-445 of SEQ ID NO: 4.

In another embodiment, a Clostridial toxin enzymatic domain comprises a BoNT/E enzymatic domain. In an aspect of this embodiment, a BoNT/E enzymatic domain comprises amino acids 1-422 of SEQ ID NO: 5. In another aspect of this embodiment, a BoNT/E enzymatic domain comprises a naturally occurring BoNT/E enzymatic domain variant, such as, e.g., an enzymatic domain from a BoNT/E isoform or an enzymatic domain from a BoNT/E subtype. In another aspect of this embodiment, a BoNT/E enzymatic domain comprises amino acids 1-422 of a naturally occurring BoNT/E enzymatic domain variant of SEQ ID NO: 5, such as, e.g., amino acids 1-422 of a BoNT/E isoform of SEQ ID NO: 5 or amino acids 1-422 of a BoNT/E subtype of SEQ ID NO: 5. In still another aspect of this embodiment, a BoNT/E enzymatic domain comprises a non-naturally occurring BoNT/E enzymatic domain variant, such as, e.g., a conservative BoNT/E enzymatic domain variant, a non-conservative BoNT/E enzymatic domain variant, a BoNT/E chimeric enzymatic domain, an active BoNT/E enzymatic domain fragment, or any combination thereof. In still another aspect of this embodiment, a BoNT/E enzymatic domain comprises amino acids 1-422 of a non-naturally occurring BoNT/E enzymatic domain variant of SEQ ID NO: 5, such as, e.g., amino acids 1-422 of a conservative BoNT/E enzymatic domain variant of SEQ ID NO: 5, amino acids 1-422 of a non-conservative BoNT/E enzymatic domain variant of SEQ ID NO: 5, amino acids 1-422 of an active BoNT/E enzymatic domain fragment of SEQ ID NO: 5, or any combination thereof.

In other aspects of this embodiment, a BoNT/E enzymatic domain comprises a polypeptide having an amino acid identity of, e.g., at least 70%, at least 75%, at least 80%, at least 85%, at least 90%, at least 95%, or at least 97% to amino acids 1-422 of SEQ ID NO: 5; or at most 70%, at most 75%, at most 80%, at most 85%, at most 90%, at most 95%, or at most 97% to amino acids 1-422 of SEQ ID NO: 5. In yet other aspects of this embodiment, a BoNT/E enzymatic domain comprises a polypeptide having, e.g., at least 1, 2, 3, 4, 5, 6, 7, 8, 9, 10, 20, 30, 40, 50, or 100 non-contiguous amino acid deletions, additions and/or substitutions relative to amino acids 1-422 of SEQ ID NO: 5; or at most 1, 2, 3, 4, 5, 6, 7, 8, 9, 10, 20, 30, 40, 50, or 100 non-contiguous amino acid deletions, additions and/or substitutions relative to amino acids 1-422 of SEQ ID NO: 5. In still other aspects of this embodiment, a BoNT/E enzymatic domain comprises a polypeptide having, e.g., at least 1, 2, 3, 4, 5, 6, 7, 8, 9, 10, 20, 30, 40, 50, or 100 contiguous amino acid deletions, additions and/or substitutions relative to amino acids 1-422 of SEQ ID NO: 5; or at most 1, 2, 3, 4, 5, 6, 7, 8, 9, 10, 20, 30, 40, 50, or 100 contiguous amino acid deletions, additions and/or substitutions relative to amino acids 1-422 of SEQ ID NO: 5.

In another embodiment, a Clostridial toxin enzymatic domain comprises a BoNT/F enzymatic domain. In an aspect of this embodiment, a BoNT/F enzymatic domain comprises amino acids 1-439 of SEQ ID NO: 6. In another aspect of this embodiment, a BoNT/F enzymatic domain comprises a naturally occurring BoNT/F enzymatic domain variant, such as, e.g., an enzymatic domain from a BoNT/F isoform or an enzymatic domain from a BoNT/F subtype. In another aspect of this embodiment, a BoNT/F enzymatic domain comprises amino acids 1-439 of a naturally occurring BoNT/F enzymatic domain variant of SEQ ID NO: 6, such as, e.g., amino acids 1-439 of a BoNT/F isoform of SEQ ID NO: 6 or amino acids 1-439 of a BoNT/F subtype of SEQ ID NO: 6. In still another aspect of this embodiment, a BoNT/F enzymatic domain comprises a non-naturally occurring BoNT/F enzymatic domain variant, such as, e.g., a conservative BoNT/F enzymatic domain variant, a non-conservative BoNT/F enzymatic domain variant, a BoNT/F chimeric enzymatic domain, an active BoNT/F enzymatic domain fragment, or any combination thereof. In still another aspect of this embodiment, a BoNT/F enzymatic domain comprises amino acids 1-439 of a non-naturally occurring BoNT/F enzymatic domain variant of SEQ ID NO: 6, such as, e.g., amino acids 1-439 of a conservative BoNT/F enzymatic domain variant of SEQ ID NO: 6, amino acids 1-439 of a non-conservative BoNT/F enzymatic domain variant of SEQ ID NO: 6, amino acids 1-439 of an active BoNT/F enzymatic domain fragment of SEQ ID NO: 6, or any combination thereof.

In other aspects of this embodiment, a BoNT/F enzymatic domain comprises a polypeptide having an amino acid identity of, e.g., at least 70%, at least 75%, at least 80%, at least 85%, at least 90%, at least 95%, or at least 97% to amino acids 1-439 of SEQ ID NO: 6; or at most 70%, at most 75%, at most 80%, at most 85%, at most 90%, at most 95%, or at most 97% to amino acids 1-439 of SEQ ID NO: 6. In yet other aspects of this embodiment, a BoNT/F enzymatic domain comprises a polypeptide having, e.g., at least 1, 2, 3, 4, 5, 6, 7, 8, 9, 10, 20, 30, 40, 50, or 100 non-contiguous amino acid deletions, additions and/or substitutions relative to amino acids 1-439 of SEQ ID NO: 6; or at most 1, 2, 3, 4, 5, 6, 7, 8, 9, 10, 20, 30, 40, 50, or 100 non-contiguous amino acid deletions, additions and/or substitutions relative to amino acids 1-439 of SEQ ID NO: 6. In still other aspects of this embodiment, a BoNT/F enzymatic domain comprises a polypeptide having, e.g., at least 1, 2, 3, 4, 5, 6, 7, 8, 9, 10, 20, 30, 40, 50, or 100 contiguous amino acid deletions, additions and/or substitutions relative to amino acids 1-439 of SEQ ID NO: 6; or at most 1, 2, 3, 4, 5, 6, 7, 8, 9, 10, 20, 30, 40, 50, or 100 contiguous amino acid deletions, additions and/or substitutions relative to amino acids 1-439 of SEQ ID NO: 6.

In another embodiment, a Clostridial toxin enzymatic domain comprises a BoNT/G enzymatic domain. In an aspect of this embodiment, a BoNT/G enzymatic domain comprises amino acids 1-446 of SEQ ID NO: 7. In another aspect of this embodiment, a BoNT/G enzymatic domain comprises a naturally occurring BoNT/G enzymatic domain variant, such as, e.g., an enzymatic domain from a BoNT/G isoform or an enzymatic domain from a BoNT/G subtype. In another aspect of this embodiment, a BoNT/G enzymatic domain comprises amino acids 1-446 of a naturally occurring BoNT/G enzymatic domain variant of SEQ ID NO: 7, such as, e.g., amino acids 1-446 of a BoNT/G isoform of SEQ ID NO: 7 or amino acids 1-446 of a BoNT/G subtype of SEQ ID NO: 7. In still another aspect of this embodiment, a BoNT/G enzymatic domain comprises a non-naturally occurring BoNT/G enzymatic domain variant, such as, e.g., a conservative BoNT/G enzymatic domain variant, a non-conservative BoNT/G enzymatic domain variant, a BoNT/G chimeric enzymatic domain, an active BoNT/G enzymatic domain fragment, or any combination thereof. In still another aspect of this embodiment, a BoNT/G enzymatic domain comprises amino acids 1-446 of a non-naturally occurring BoNT/G enzymatic domain variant of SEQ ID NO: 7, such as, e.g., amino acids 1-446 of a conservative BoNT/G enzymatic domain variant of SEQ ID NO: 7, amino acids 1-446 of a non-conservative BoNT/G enzymatic domain variant of SEQ ID NO: 7, amino acids 1-446 of an active BoNT/G enzymatic domain fragment of SEQ ID NO: 7, or any combination thereof.

In other aspects of this embodiment, a BoNT/G enzymatic domain comprises a polypeptide having an amino acid identity of, e.g., at least 70%, at least 75%, at least 80%, at least 85%, at least 90%, at least 95%, or at least 97% to amino acids 1-446 of SEQ ID NO: 7; or at most 70%, at most 75%, at most 80%, at most 85%, at most 90%, at most 95%, or at most 97% to amino acids 1-446 of SEQ ID NO: 7. In yet other aspects of this embodiment, a BoNT/G enzymatic domain comprises a polypeptide having, e.g., at least 1, 2, 3, 4, 5, 6, 7, 8, 9, 10, 20, 30, 40, 50, or 100 non-contiguous amino acid deletions, additions and/or substitutions relative to amino acids 1-446 of SEQ ID NO: 7; or at most 1, 2, 3, 4, 5, 6, 7, 8, 9, 10, 20, 30, 40, 50, or 100 non-contiguous amino acid deletions, additions and/or substitutions relative to amino acids 1-446 of SEQ ID NO: 7. In still other aspects of this embodiment, a BoNT/G enzymatic domain comprises a polypeptide having, e.g., at least 1, 2, 3, 4, 5, 6, 7, 8, 9, 10, 20, 30, 40, 50, or 100 contiguous amino acid deletions, additions and/or substitutions relative to amino acids 1-446 of SEQ ID NO: 7; or at most 1, 2, 3, 4, 5, 6, 7, 8, 9, 10, 20, 30, 40, 50, or 100 contiguous amino acid deletions, additions and/or substitutions relative to amino acids 1-446 of SEQ ID NO: 7.

In another embodiment, a Clostridial toxin enzymatic domain comprises a TeNT enzymatic domain. In an aspect of this embodiment, a TeNT enzymatic domain comprises amino acids 1-457 of SEQ ID NO: 8. In another aspect of this embodiment, a TeNT enzymatic domain comprises a naturally occurring TeNT enzymatic domain variant, such as, e.g., an enzymatic domain from a TeNT isoform or an enzymatic domain from a TeNT subtype. In another aspect of this embodiment, a TeNT enzymatic domain comprises amino acids 1-457 of a naturally occurring TeNT enzymatic domain variant of SEQ ID NO: 8, such as, e.g., amino acids 1-457 of a TeNT isoform of SEQ ID NO: 8 or amino acids 1-457 of a TeNT subtype of SEQ ID NO: 8. In still another aspect of this embodiment, a TeNT enzymatic domain comprises a non-naturally occurring TeNT enzymatic domain variant, such as, e.g., a conservative TeNT enzymatic domain variant, a non-conservative TeNT enzymatic domain variant, a TeNT chimeric enzymatic domain, an active TeNT enzymatic domain fragment, or any combination thereof. In still another aspect of this embodiment, a TeNT enzymatic domain comprises amino acids 1-457 of a non-naturally occurring TeNT enzymatic domain variant of SEQ ID NO: 8, such as, e.g., amino acids 1-457 of a conservative TeNT enzymatic domain variant of SEQ ID NO: 8, amino acids 1-457 of a non-conservative TeNT enzymatic domain variant of SEQ ID NO: 8, amino acids 1-457 of an active TeNT enzymatic domain fragment of SEQ ID NO: 8, or any combination thereof.

In other aspects of this embodiment, a TeNT enzymatic domain comprises a polypeptide having an amino acid identity of, e.g., at least 70%, at least 75%, at least 80%, at least 85%, at least 90%, at least 95%, or at least 97% to amino acids 1-457 of SEQ ID NO: 8; or at most 70%, at most 75%, at most 80%, at most 85%, at most 90%, at most 95%, or at most 97% to amino acids 1-457 of SEQ ID NO: 8. In yet other aspects of this embodiment, a TeNT enzymatic domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 1-457 of SEQ ID NO: 8; or at most 1, 2, 3, 4, 5, 6, 7, 8, 9, 10, 20, 30, 40, 50, or 100 non-contiguous amino acid deletions, additions, and/or substitutions relative to amino acids 1-457 of SEQ ID NO: 8. In still other aspects of this embodiment, a TeNT enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 1-457 of SEQ ID NO: 8; at most 1, 2, 3, 4, 5, 6, 7, 8, 9, 10, 20, 30, 40, 50, or 100 contiguous amino acid substitutions relative to amino acids 1-457 of SEQ ID NO: 8.

In another embodiment, a Clostridial toxin enzymatic domain comprises a BaNT enzymatic domain. In an aspect of this embodiment, a BaNT enzymatic domain comprises amino acids 1-431 of SEQ ID NO: 9. In another aspect of this embodiment, a BaNT enzymatic domain comprises a naturally occurring BaNT enzymatic domain variant, such as, e.g., an enzymatic domain from a BaNT isoform or an enzymatic domain from a BaNT subtype. In another aspect of this embodiment, a BaNT enzymatic domain comprises amino acids 1-431 of a naturally occurring BaNT enzymatic domain variant of SEQ ID NO: 9, such as, e.g., amino acids 1-431 of a BaNT isoform of SEQ ID NO: 9 or amino acids 1-431 of a BaNT subtype of SEQ ID NO: 9. In still another aspect of this embodiment, a BaNT enzymatic domain comprises a non-naturally occurring BaNT enzymatic domain variant, such as, e.g., a conservative BaNT enzymatic domain variant, a non-conservative BaNT enzymatic domain variant, a BaNT chimeric enzymatic domain, an active BaNT enzymatic domain fragment, or any combination thereof. In still another aspect of this embodiment, a BaNT enzymatic domain comprises amino acids 1-431 of a non-naturally occurring BaNT enzymatic domain variant of SEQ ID NO: 9, such as, e.g., amino acids 1-431 of a conservative BaNT enzymatic domain variant of SEQ ID NO: 9, amino acids 1-431 of a non-conservative BaNT enzymatic domain variant of SEQ ID NO: 9, amino acids 1-431 of an active BaNT enzymatic domain fragment of SEQ ID NO: 9, or any combination thereof.

In other aspects of this embodiment, a BaNT enzymatic domain comprises a polypeptide having an amino acid identity of, e.g., at least 70%, at least 75%, at least 80%, at least 85%, at least 90%, at least 95%, or at least 97% to amino acids 1-431 of SEQ ID NO: 9; or at most 70%, at most 75%, at most 80%, at most 85%, at most 90%, at most 95%, or at most 97% to amino acids 1-431 of SEQ ID NO: 9. In yet other aspects of this embodiment, a BaNT enzymatic domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 1-431 of SEQ ID NO: 9; or at most 1, 2, 3, 4, 5, 6, 7, 8, 9, 10, 20, 30, 40, 50, or 100 non-contiguous amino acid deletions, additions, and/or substitutions relative to amino acids 1-431 of SEQ ID NO: 9. In still other aspects of this embodiment, a BaNT enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 1-431 of SEQ ID NO: 9; at most 1, 2, 3, 4, 5, 6, 7, 8, 9, 10, 20, 30, 40, 50, or 100 contiguous amino acid deletions, additions, and/or substitutions relative to amino acids 1-431 of SEQ ID NO: 9.

In another embodiment, a Clostridial toxin enzymatic domain comprises a BuNT enzymatic domain. In an aspect of this embodiment, a BuNT enzymatic domain comprises amino acids 1-422 of SEQ ID NO: 10. In another aspect of this embodiment, a BuNT enzymatic domain comprises a naturally occurring BuNT enzymatic domain variant, such as, e.g., an enzymatic domain from a BuNT isoform or an enzymatic domain from a BuNT subtype. In another aspect of this embodiment, a BuNT enzymatic domain comprises amino acids 1-422 of a naturally occurring BuNT enzymatic domain variant of SEQ ID NO: 10, such as, e.g., amino acids 1-422 of a BuNT isoform of SEQ ID NO: 10 or amino acids 1-422 of a BuNT subtype of SEQ ID NO: 10. In still another aspect of this embodiment, a BuNT enzymatic domain comprises a non-naturally occurring BuNT enzymatic domain variant, such as, e.g., a conservative BuNT enzymatic domain variant, a non-conservative BuNT enzymatic domain variant, a BuNT chimeric enzymatic domain, an active BuNT enzymatic domain fragment, or any combination thereof. In still another aspect of this embodiment, a BuNT enzymatic domain comprises amino acids 1-422 of a non-naturally occurring BuNT enzymatic domain variant of SEQ ID NO: 10, such as, e.g., amino acids 1-422 of a conservative BuNT enzymatic domain variant of SEQ ID NO: 10, amino acids 1-422 of a non-conservative BuNT enzymatic domain variant of SEQ ID NO: 10, amino acids 1-422 of an active BuNT enzymatic domain fragment of SEQ ID NO: 10, or any combination thereof.

In other aspects of this embodiment, a BuNT enzymatic domain comprises a polypeptide having an amino acid identity of, e.g., at least 70%, at least 75%, at least 80%, at least 85%, at least 90%, at least 95%, or at least 97% to amino acids 1-422 of SEQ ID NO: 10; or at most 70%, at most 75%, at most 80%, at most 85%, at most 90%, at most 95%, or at most 97% to amino acids 1-422 of SEQ ID NO: 10. In yet other aspects of this embodiment, a BuNT enzymatic domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 1-422 of SEQ ID NO: 1; or at most 1, 2, 3, 4, 5, 6, 7, 8, 9, 10, 20, 30, 40, 50, or 100 non-contiguous amino acid deletions, additions, and/or substitutions relative to amino acids 1-422 of SEQ ID NO: 10. In still other aspects of this embodiment, a BuNT enzymatic domain comprises a polypeptide having, e.g., at least 1, 2, 3, 4, 5, 6, 7, 8, 9, 10, 20, 30, 40, 50, 100 or 200 contiguous amino acid deletions, additions, and/or substitutions relative to amino acids 1-422 of SEQ ID NO: 10; or at most 1, 2, 3, 4, 5, 6, 7, 8, 9, 10, 20, 30, 40, 50, 100 or 200 contiguous amino acid deletions, additions, and/or substitutions relative to amino acids 1-422 of SEQ ID NO: 10.

The “translocation domain” comprises a portion of a Clostridial neurotoxin heavy chain having a translocation activity. By “translocation” is meant the ability to facilitate the transport of a polypeptide through a vesicular membrane, thereby exposing some or all of the polypeptide to the cytoplasm. In the various botulinum neurotoxins translocation is thought to involve an allosteric conformational change of the heavy chain caused by a decrease in pH within the endosome. This conformational change appears to involve and be mediated by the N terminal half of the heavy chain and to result in the formation of pores in the vesicular membrane; this change permits the movement of the proteolytic light chain from within the endosomal vesicle into the cytoplasm. See e.g., Lacy, et al., Nature Struct. Biol. 5:898-902 (October 1998).

The amino acid sequence of the translocation-mediating portion of the botulinum neurotoxin heavy chain is known to those of skill in the art; additionally, those amino acid residues within this portion that are known to be essential for conferring the translocation activity are also known. It would therefore be well within the ability of one of ordinary skill in the art, for example, to employ the naturally occurring N-terminal peptide half of the heavy chain of any of the various Clostridium tetanus or Clostridium botulinum neurotoxin subtypes as a translocation domain, or to design an analogous translocation domain by aligning the primary sequences of the N-terminal halves of the various heavy chains and selecting a consensus primary translocation sequence based on conserved amino acid, polarity, steric and hydrophobicity characteristics between the sequences.

In another aspect of the invention, a TVEMP comprises, in part, a Clostridial toxin translocation domain. As used herein, the term “Clostridial toxin translocation domain” refers to any Clostridial toxin polypeptide that can execute the translocation step of the intoxication process that mediates Clostridial toxin light chain translocation. Thus, a Clostridial toxin translocation domain facilitates the movement of a Clostridial toxin light chain across a membrane and encompasses the movement of a Clostridial toxin light chain through the membrane an intracellular vesicle into the cytoplasm of a cell. Non-limiting examples of a Clostridial toxin translocation domain include, e.g., a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, and a BuNT translocation domain. Other non-limiting examples of a Clostridial toxin translocation domain include, e.g., amino acids 449-873 of SEQ ID NO: 1, amino acids 442-860 of SEQ ID NO: 2, amino acids 450-868 of SEQ ID NO: 3, amino acids 446-864 of SEQ ID NO: 4, amino acids 423-847 of SEQ ID NO: 5, amino acids 440-866 of SEQ ID NO: 6, amino acids 447-865 of SEQ ID NO: 7, amino acids 458-881 of SEQ ID NO: 8, amino acids 432-857 of SEQ ID NO: 9, and amino acids 423-847 of SEQ ID NO: 10.

A Clostridial toxin translocation domain includes, without limitation, naturally occurring Clostridial toxin translocation domain variants, such as, e.g., Clostridial toxin translocation domain isoforms and Clostridial toxin translocation domain subtypes; non-naturally occurring Clostridial toxin translocation domain variants, such as, e.g., conservative Clostridial toxin translocation domain variants, non-conservative Clostridial toxin translocation domain variants, Clostridial toxin translocation domain chimerics, active Clostridial toxin translocation domain fragments thereof, or any combination thereof.

As used herein, the term “Clostridial toxin translocation domain variant,” whether naturally-occurring or non-naturally-occurring, refers to a Clostridial toxin translocation domain that has at least one amino acid change from the corresponding region of the disclosed reference sequences (Table 1) and can be described in percent identity to the corresponding region of that reference sequence. Unless expressly indicated, Clostridial toxin translocation domain variants useful to practice disclosed embodiments are variants that execute the translocation step of the intoxication process that mediates Clostridial toxin light chain translocation. As non-limiting examples, a BoNT/A translocation domain variant comprising amino acids 449-873 of SEQ ID NO: 1 will have at least one amino acid difference, such as, e.g., an amino acid substitution, deletion or addition, as compared to the amino acid region 449-873 of SEQ ID NO: 1; a BoNT/B translocation domain variant comprising amino acids 442-860 of SEQ ID NO: 2 will have at least one amino acid difference, such as, e.g., an amino acid substitution, deletion or addition, as compared to the amino acid region 442-860 of SEQ ID NO: 2; a BoNT/C1 translocation domain variant comprising amino acids 450-868 of SEQ ID NO: 3 will have at least one amino acid difference, such as, e.g., an amino acid substitution, deletion or addition, as compared to the amino acid region 450-868 of SEQ ID NO: 3; a BoNT/D translocation domain variant comprising amino acids 446-864 of SEQ ID NO: 4 will have at least one amino acid difference, such as, e.g., an amino acid substitution, deletion or addition, as compared to the amino acid region 446-864 of SEQ ID NO: 4; a BoNT/E translocation domain variant comprising amino acids 423-847 of SEQ ID NO: 5 will have at least one amino acid difference, such as, e.g., an amino acid substitution, deletion or addition, as compared to the amino acid region 423-847 of SEQ ID NO: 5; a BoNT/F translocation domain variant comprising amino acids 440-866 of SEQ ID NO: 6 will have at least one amino acid difference, such as, e.g., an amino acid substitution, deletion or addition, as compared to the amino acid region 440-866 of SEQ ID NO: 6; a BoNT/G translocation domain variant comprising amino acids 447-865 of SEQ ID NO: 7 will have at least one amino acid difference, such as, e.g., an amino acid substitution, deletion or addition, as compared to the amino acid region 447-865 of SEQ ID NO: 7; a TeNT translocation domain variant comprising amino acids 458-881 of SEQ ID NO: 8 will have at least one amino acid difference, such as, e.g., an amino acid substitution, deletion or addition, as compared to the amino acid region 458-881 of SEQ ID NO: 8; a BaNT translocation domain variant comprising amino acids 432-857 of SEQ ID NO: 9 will have at least one amino acid difference, such as, e.g., an amino acid substitution, deletion or addition, as compared to the amino acid region 432-857 of SEQ ID NO: 9; and a BuNT translocation domain variant comprising amino acids 423-847 of SEQ ID NO: 10 will have at least one amino acid difference, such as, e.g., an amino acid substitution, deletion or addition, as compared to the amino acid region 423-847 of SEQ ID NO: 10.

It is recognized by those of skill in the art that within each serotype of Clostridial toxin there can be naturally occurring Clostridial toxin translocation domain variants that differ somewhat in their amino acid sequence, and also in the nucleic acids encoding these proteins. For example, there are presently five BoNT/A subtypes, BoNT/A1, BoNT/A2, BoNT/A3, BoNT/A4, and BoNT/A5, with specific translocation domain subtypes showing approximately 87% amino acid identity when compared to another BoNT/A translocation domain subtype. As used herein, the term “naturally occurring Clostridial toxin translocation domain variant” refers to any Clostridial toxin translocation domain produced by a naturally-occurring process, including, without limitation, Clostridial toxin translocation domain isoforms produced from alternatively-spliced transcripts, Clostridial toxin translocation domain isoforms produced by spontaneous mutation and Clostridial toxin translocation domain subtypes. A naturally occurring Clostridial toxin translocation domain variant can function in substantially the same manner as the reference Clostridial toxin translocation domain on which the naturally occurring Clostridial toxin translocation domain variant is based, and can be substituted for the reference Clostridial toxin translocation domain in any aspect of the present invention.

A non-limiting example of a naturally occurring Clostridial toxin translocation domain variant is a Clostridial toxin translocation domain isoform such as, e.g., a BoNT/A translocation domain isoform, a BoNT/B translocation domain isoform, a BoNT/C1 translocation domain isoform, a BoNT/D translocation domain isoform, a BoNT/E translocation domain isoform, a BoNT/F translocation domain isoform, a BoNT/G translocation domain isoform, a TeNT translocation domain isoform, a BaNT translocation domain isoform, and a BuNT translocation domain isoform. Another non-limiting example of a naturally occurring Clostridial toxin translocation domain variant is a Clostridial toxin translocation domain subtype such as, e.g., a translocation domain from subtype BoNT/A1, BoNT/A2, BoNT/A3, BoNT/A4, and BoNT/A5; a translocation domain from subtype BoNT/B1, BoNT/B2, BoNT/B bivalent and BoNT/B nonproteolytic; a translocation domain from subtype BoNT/C1-1 and BoNT/C1-2; a translocation domain from subtype BoNT/E1, BoNT/E2 and BoNT/E3; and a translocation domain from subtype BoNT/F1, BoNT/F2, BoNT/F3 and BoNT/F4.

As used herein, the term “non-naturally occurring Clostridial toxin translocation domain variant” refers to any Clostridial toxin translocation domain produced with the aid of human manipulation, including, without limitation, Clostridial toxin translocation domains produced by genetic engineering using random mutagenesis or rational design and Clostridial toxin translocation domains produced by chemical synthesis. Non-limiting examples of non-naturally occurring Clostridial toxin translocation domain variants include, e.g., conservative Clostridial toxin translocation domain variants, non-conservative Clostridial toxin translocation domain variants, Clostridial toxin translocation domain chimeric variants and active Clostridial toxin translocation domain fragments.

As used herein, the term “conservative Clostridial toxin translocation domain variant” refers to a Clostridial toxin translocation domain that has at least one amino acid substituted by another amino acid or an amino acid analog that has at least one property similar to that of the original amino acid from the reference Clostridial toxin translocation domain sequence (Table 1). Examples of properties include, without limitation, similar size, topography, charge, hydrophobicity, hydrophilicity, lipophilicity, covalent-bonding capacity, hydrogen-bonding capacity, a physicochemical property, of the like, or any combination thereof. A conservative Clostridial toxin translocation domain variant can function in substantially the same manner as the reference Clostridial toxin translocation domain on which the conservative Clostridial toxin translocation domain variant is based, and can be substituted for the reference Clostridial toxin translocation domain in any aspect of the present invention. Non-limiting examples of a conservative Clostridial toxin translocation domain variant include, e.g., conservative BoNT/A translocation domain variants, conservative BoNT/B translocation domain variants, conservative BoNT/C1 translocation domain variants, conservative BoNT/D translocation domain variants, conservative BoNT/E translocation domain variants, conservative BoNT/F translocation domain variants, conservative BoNT/G translocation domain variants, conservative TeNT translocation domain variants, conservative BaNT translocation domain variants, and conservative BuNT translocation domain variants.

As used herein, the term “non-conservative Clostridial toxin translocation domain variant” refers to a Clostridial toxin translocation domain in which 1) at least one amino acid is deleted from the reference Clostridial toxin translocation domain on which the non-conservative Clostridial toxin translocation domain variant is based; 2) at least one amino acid added to the reference Clostridial toxin translocation domain on which the non-conservative Clostridial toxin translocation domain is based; or 3) at least one amino acid is substituted by another amino acid or an amino acid analog that does not share any property similar to that of the original amino acid from the reference Clostridial toxin translocation domain sequence (Table 1). A non-conservative Clostridial toxin translocation domain variant can function in substantially the same manner as the reference Clostridial toxin translocation domain on which the non-conservative Clostridial toxin translocation domain variant is based, and can be substituted for the reference Clostridial toxin translocation domain in any aspect of the present invention. Non-limiting examples of a non-conservative Clostridial toxin translocation domain variant include, e.g., non-conservative BoNT/A translocation domain variants, non-conservative BoNT/B translocation domain variants, non-conservative BoNT/C1 translocation domain variants, non-conservative BoNT/D translocation domain variants, non-conservative BoNT/E translocation domain variants, non-conservative BoNT/F translocation domain variants, non-conservative BoNT/G translocation domain variants, and non-conservative TeNT translocation domain variants, non-conservative BaNT translocation domain variants, and non-conservative BuNT translocation domain variants.

As used herein, the term “Clostridial toxin translocation domain chimeric” refers to a polypeptide comprising at least a portion of a Clostridial toxin translocation domain and at least a portion of at least one other polypeptide to form a toxin translocation domain with at least one property different from the reference Clostridial toxin translocation domains of Table 1, with the proviso that this Clostridial toxin translocation domain chimeric is still capable of specifically targeting the core components of the neurotransmitter release apparatus and thus participate in executing the overall cellular mechanism whereby a Clostridial toxin proteolytically cleaves a substrate.

As used herein, the term “active Clostridial toxin translocation domain fragment” refers to any of a variety of Clostridial toxin fragments comprising the translocation domain can be useful in aspects of the present invention with the proviso that these active fragments can facilitate the release of the LC from intracellular vesicles into the cytoplasm of the target cell and thus participate in executing the overall cellular mechanism whereby a Clostridial toxin proteolytically cleaves a substrate. The translocation domains from the heavy chains of Clostridial toxins are approximately 410-430 amino acids in length and comprise a translocation domain (Table 1). Research has shown that the entire length of a translocation domain from a Clostridial toxin heavy chain is not necessary for the translocating activity of the translocation domain. Thus, aspects of this embodiment can include Clostridial toxin translocation domains comprising a translocation domain having a length of, e.g., at least 350 amino acids, at least 375 amino acids, at least 400 amino acids and at least 425 amino acids. Other aspects of this embodiment can include Clostridial toxin translocation domains comprising translocation domain having a length of, e.g., at most 350 amino acids, at most 375 amino acids, at most 400 amino acids and at most 425 amino acids.

Any of a variety of sequence alignment methods can be used to determine percent identity of naturally-occurring Clostridial toxin translocation domain variants and non-naturally-occurring Clostridial toxin translocation domain variants, including, without limitation, global methods, local methods and hybrid methods, such as, e.g., segment approach methods. Protocols to determine percent identity are routine procedures within the scope of one skilled in the art and from the teaching herein.

Thus, in an embodiment, a TVEMP disclosed in the present specification comprises a Clostridial toxin translocation domain. In an aspect of this embodiment, a Clostridial toxin translocation domain comprises a naturally occurring Clostridial toxin translocation domain variant, such as, e.g., a Clostridial toxin translocation domain isoform or a Clostridial toxin translocation domain subtype. In another aspect of this embodiment, a Clostridial toxin translocation domain comprises a non-naturally occurring Clostridial toxin translocation domain variant, such as, e.g., a conservative Clostridial toxin translocation domain variant, a non-conservative Clostridial toxin translocation domain variant, a Clostridial toxin chimeric translocation domain, an active Clostridial toxin translocation domain fragment, or any combination thereof.

In another embodiment, a Clostridial toxin translocation domain comprises a BoNT/A translocation domain. In an aspect of this embodiment, a BoNT/A translocation domain comprises amino acids 449-873 of SEQ ID NO: 1. In another aspect of this embodiment, a BoNT/A translocation domain comprises a naturally occurring BoNT/A translocation domain variant, such as, e.g., a translocation domain from a BoNT/A isoform or a translocation domain from a BoNT/A subtype. In another aspect of this embodiment, a BoNT/A translocation domain comprises amino acids 449-873 of a naturally occurring BoNT/A translocation domain variant of SEQ ID NO: 1, such as, e.g., amino acids 449-873 of a BoNT/A isoform of SEQ ID NO: 1 or amino acids 449-873 of a BoNT/A subtype of SEQ ID NO: 1. In still another aspect of this embodiment, a BoNT/A translocation domain comprises a non-naturally occurring BoNT/A translocation domain variant, such as, e.g., a conservative BoNT/A translocation domain variant, a non-conservative BoNT/A translocation domain variant, a BoNT/A chimeric translocation domain, an active BoNT/A translocation domain fragment, or any combination thereof. In still another aspect of this embodiment, a BoNT/A translocation domain comprises amino acids 449-873 of a non-naturally occurring BoNT/A translocation domain variant of SEQ ID NO: 1, such as, e.g., amino acids 449-873 of a conservative BoNT/A translocation domain variant of SEQ ID NO: 1, amino acids 449-873 of a non-conservative BoNT/A translocation domain variant of SEQ ID NO: 1, amino acids 449-873 of an active BoNT/A translocation domain fragment of SEQ ID NO: 1, or any combination thereof.

In other aspects of this embodiment, a BoNT/A translocation domain comprises a polypeptide having an amino acid identity of, e.g., at least 70%, at least 75%, at least 80%, at least 85%, at least 90%, at least 95%, or at least 97% to amino acids 449-873 of SEQ ID NO: 1; or at most 70%, at most 75%, at most 80%, at most 85%, at most 90%, at most 95%, or at most 97% to amino acids 449-873 of SEQ ID NO: 1. In yet other aspects of this embodiment, a BoNT/A translocation domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 449-873 of SEQ ID NO: 1; at most 1, 2, 3, 4, 5, 6, 7, 8, 9, 10, 20, 30, 40, 50, or 100 non-contiguous amino acid deletions, additions, and/or substitutions relative to amino acids 449-873 of SEQ ID NO: 1. In still other aspects of this embodiment, a BoNT/A translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 449-873 of SEQ ID NO: 1; at most 1, 2, 3, 4, 5, 6, 7, 8, 9, 10, 20, 30, 40, 50, 100 or 200 contiguous amino acid deletions, additions, and/or substitutions relative to amino acids 449-873 of SEQ ID NO: 1.

In another embodiment, a Clostridial toxin translocation domain comprises a BoNT/B translocation domain. In an aspect of this embodiment, a BoNT/B translocation domain comprises amino acids 442-860 of SEQ ID NO: 2. In another aspect of this embodiment, a BoNT/B translocation domain comprises a naturally occurring BoNT/B translocation domain variant, such as, e.g., a translocation domain from a BoNT/B isoform or a translocation domain from a BoNT/B subtype. In another aspect of this embodiment, a BoNT/B translocation domain comprises amino acids 442-860 of a naturally occurring BoNT/B translocation domain variant of SEQ ID NO: 2, such as, e.g., amino acids 442-860 of a BoNT/B isoform of SEQ ID NO: 2 or amino acids 442-860 of a BoNT/B subtype of SEQ ID NO: 2. In still another aspect of this embodiment, a BoNT/B translocation domain comprises a non-naturally occurring BoNT/B translocation domain variant, such as, e.g., a conservative BoNT/B translocation domain variant, a non-conservative BoNT/B translocation domain variant, a BoNT/B chimeric translocation domain, an active BoNT/B translocation domain fragment, or any combination thereof. In still another aspect of this embodiment, a BoNT/B translocation domain comprises amino acids 442-860 of a non-naturally occurring BoNT/B translocation domain variant of SEQ ID NO: 2, such as, e.g., amino acids 442-860 of a conservative BoNT/B translocation domain variant of SEQ ID NO: 2, amino acids 442-860 of a non-conservative BoNT/B translocation domain variant of SEQ ID NO: 2, amino acids 442-860 of an active BoNT/B translocation domain fragment of SEQ ID NO: 2, or any combination thereof.

In other aspects of this embodiment, a BoNT/B translocation domain comprises a polypeptide having an amino acid identity of, e.g., at least 70%, at least 75%, at least 80%, at least 85%, at least 90%, at least 95%, or at least 97% to amino acids 442-860 of SEQ ID NO: 2; or at most 70%, at most 75%, at most 80%, at most 85%, at most 90%, at most 95%, or at most 97% to amino acids 442-860 of SEQ ID NO: 2. In yet other aspects of this embodiment, a BoNT/B translocation domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 442-860 of SEQ ID NO: 2; or at most 1, 2, 3, 4, 5, 6, 7, 8, 9, 10, 20, 30, 40, 50, 100 or 200 non-contiguous amino acid deletions, additions, and/or substitutions relative to amino acids 442-860 of SEQ ID NO: 2. In still other aspects of this embodiment, a BoNT/B translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 442-860 of SEQ ID NO: 2; or at most 1, 2, 3, 4, 5, 6, 7, 8, 9, 10, 20, 30, 40, 50, or 100 contiguous amino acid deletions, additions, and/or substitutions relative to amino acids 442-860 of SEQ ID NO: 2.

In another embodiment, a Clostridial toxin translocation domain comprises a BoNT/C1 translocation domain. In an aspect of this embodiment, a BoNT/C1 translocation domain comprises amino acids 450-868 of SEQ ID NO: 3. In another aspect of this embodiment, a BoNT/C1 translocation domain comprises a naturally occurring BoNT/C1 translocation domain variant, such as, e.g., a translocation domain from a BoNT/C1 isoform or a translocation domain from a BoNT/C1 subtype. In another aspect of this embodiment, a BoNT/C1 translocation domain comprises amino acids 450-868 of a naturally occurring BoNT/C1 translocation domain variant of SEQ ID NO: 3, such as, e.g., amino acids 450-868 of a BoNT/C1 isoform of SEQ ID NO: 3 or amino acids 450-868 of a BoNT/C1 subtype of SEQ ID NO: 3. In still another aspect of this embodiment, a BoNT/C1 translocation domain comprises a non-naturally occurring BoNT/C1 translocation domain variant, such as, e.g., a conservative BoNT/C1 translocation domain variant, a non-conservative BoNT/C1 translocation domain variant, a BoNT/C1 chimeric translocation domain, an active BoNT/C1 translocation domain fragment, or any combination thereof. In still another aspect of this embodiment, a BoNT/C1 translocation domain comprises amino acids 450-868 of a non-naturally occurring BoNT/C1 translocation domain variant of SEQ ID NO: 3, such as, e.g., amino acids 450-868 of a conservative BoNT/C1 translocation domain variant of SEQ ID NO: 3, amino acids 450-868 of a non-conservative BoNT/C1 translocation domain variant of SEQ ID NO: 3, amino acids 450-868 of an active BoNT/C1 translocation domain fragment of SEQ ID NO: 3, or any combination thereof.

In other aspects of this embodiment, a BoNT/C1 translocation domain comprises a polypeptide having an amino acid identity of, e.g., at least 70%, at least 75%, at least 80%, at least 85%, at least 90%, at least 95%, or at least 97% to amino acids 450-868 of SEQ ID NO: 3; or at most 70%, at most 75%, at most 80%, at most 85%, at most 90%, at most 95%, or at most 97% to amino acids 450-868 of SEQ ID NO: 3. In yet other aspects of this embodiment, a BoNT/C1 translocation domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 450-868 of SEQ ID NO: 3; or at most 1, 2, 3, 4, 5, 6, 7, 8, 9, 10, 20, 30, 40, 50, or 100 non-contiguous amino acid deletions, additions, and/or substitutions relative to amino acids 450-868 of SEQ ID NO: 3. In still other aspects of this embodiment, a BoNT/C1 translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 450-868 of SEQ ID NO: 3; or at most 1, 2, 3, 4, 5, 6, 7, 8, 9, 10, 20, 30, 40, 50, or 100 contiguous amino acid deletions, additions, and/or substitutions relative to amino acids 450-868 of SEQ ID NO: 3.

In another embodiment, a Clostridial toxin translocation domain comprises a BoNT/D translocation domain. In an aspect of this embodiment, a BoNT/D translocation domain comprises amino acids 446-864 of SEQ ID NO: 4. In another aspect of this embodiment, a BoNT/D translocation domain comprises a naturally occurring BoNT/D translocation domain variant, such as, e.g., a translocation domain from a BoNT/D isoform or a translocation domain from a BoNT/D subtype. In another aspect of this embodiment, a BoNT/D translocation domain comprises amino acids 446-864 of a naturally occurring BoNT/D translocation domain variant of SEQ ID NO: 4, such as, e.g., amino acids 446-864 of a BoNT/D isoform of SEQ ID NO: 4 or amino acids 446-864 of a BoNT/D subtype of SEQ ID NO: 4. In still another aspect of this embodiment, a BoNT/D translocation domain comprises a non-naturally occurring BoNT/D translocation domain variant, such as, e.g., a conservative BoNT/D translocation domain variant, a non-conservative BoNT/D translocation domain variant, a BoNT/D chimeric translocation domain, an active BoNT/D translocation domain fragment, or any combination thereof. In still another aspect of this embodiment, a BoNT/D translocation domain comprises amino acids 446-864 of a non-naturally occurring BoNT/D translocation domain variant of SEQ ID NO: 4, such as, e.g., amino acids 446-864 of a conservative BoNT/D translocation domain variant of SEQ ID NO: 4, amino acids 446-864 of a non-conservative BoNT/D translocation domain variant of SEQ ID NO: 4, amino acids 446-864 of an active BoNT/D translocation domain fragment of SEQ ID NO: 4, or any combination thereof.

In other aspects of this embodiment, a BoNT/D translocation domain comprises a polypeptide having an amino acid identity of, e.g., at least 70%, at least 75%, at least 80%, at least 85%, at least 90%, at least 95%, or at least 97% to amino acids 446-864 of SEQ ID NO: 4; or at most 70%, at most 75%, at most 80%, at most 85%, at most 90%, at most 95%, or at most 97% to amino acids 446-864 of SEQ ID NO: 4. In yet other aspects of this embodiment, a BoNT/D translocation domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 446-864 of SEQ ID NO: 4; or at most 1, 2, 3, 4, 5, 6, 7, 8, 9, 10, 20, 30, 40, 50, or 100 non-contiguous amino acid deletions, additions, and/or substitutions relative to amino acids 446-864 of SEQ ID NO: 4. In still other aspects of this embodiment, a BoNT/D translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 446-864 of SEQ ID NO: 4; or at most 1, 2, 3, 4, 5, 6, 7, 8, 9, 10, 20, 30, 40, 50, or 100 contiguous amino acid substitutions relative to amino acids 446-864 of SEQ ID NO: 4.

In another embodiment, a Clostridial toxin translocation domain comprises a BoNT/E translocation domain. In an aspect of this embodiment, a BoNT/E translocation domain comprises amino acids 423-847 of SEQ ID NO: 5. In another aspect of this embodiment, a BoNT/E translocation domain comprises a naturally occurring BoNT/E translocation domain variant, such as, e.g., a translocation domain from a BoNT/E isoform or a translocation domain from a BoNT/E subtype. In another aspect of this embodiment, a BoNT/E translocation domain comprises amino acids 423-847 of a naturally occurring BoNT/E translocation domain variant of SEQ ID NO: 5, such as, e.g., amino acids 423-847 of a BoNT/E isoform of SEQ ID NO: 5 or amino acids 423-847 of a BoNT/E subtype of SEQ ID NO: 5. In still another aspect of this embodiment, a BoNT/E translocation domain comprises a non-naturally occurring BoNT/E translocation domain variant, such as, e.g., a conservative BoNT/E translocation domain variant, a non-conservative BoNT/E translocation domain variant, a BoNT/E chimeric translocation domain, an active BoNT/E translocation domain fragment, or any combination thereof. In still another aspect of this embodiment, a BoNT/E translocation domain comprises amino acids 423-847 of a non-naturally occurring BoNT/E translocation domain variant of SEQ ID NO: 5, such as, e.g., amino acids 423-847 of a conservative BoNT/E translocation domain variant of SEQ ID NO: 5, amino acids 423-847 of a non-conservative BoNT/E translocation domain variant of SEQ ID NO: 5, amino acids 423-847 of an active BoNT/E translocation domain fragment of SEQ ID NO: 5, or any combination thereof.

In other aspects of this embodiment, a BoNT/E translocation domain comprises a polypeptide having an amino acid identity of, e.g., at least 70%, at least 75%, at least 80%, at least 85%, at least 90%, at least 95%, or at least 97% to amino acids 423-847 of SEQ ID NO: 5; or at most 70%, at most 75%, at most 80%, at most 85%, at most 90%, at most 95%, or at most 97% to amino acids 423-847 of SEQ ID NO: 5. In yet other aspects of this embodiment, a BoNT/E translocation domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 423-847 of SEQ ID NO: 5; or at most 1, 2, 3, 4, 5, 6, 7, 8, 9, 10, 20, 30, 40, 50, or 100 non-contiguous amino acid deletions, additions, and/or substitutions relative to amino acids 423-847 of SEQ ID NO: 5. In still other aspects of this embodiment, a BoNT/E translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 423-847 of SEQ ID NO: 5; or at most 1, 2, 3, 4, 5, 6, 7, 8, 9, 10, 20, 30, 40, 50, or 100 contiguous amino acid substitutions relative to amino acids 423-847 of SEQ ID NO: 5.

In another embodiment, a Clostridial toxin translocation domain comprises a BoNT/F translocation domain. In an aspect of this embodiment, a BoNT/F translocation domain comprises amino acids 440-866 of SEQ ID NO: 6. In another aspect of this embodiment, a BoNT/F translocation domain comprises a naturally occurring BoNT/F translocation domain variant, such as, e.g., a translocation domain from a BoNT/F isoform or a translocation domain from a BoNT/F subtype. In another aspect of this embodiment, a BoNT/F translocation domain comprises amino acids 440-866 of a naturally occurring BoNT/F translocation domain variant of SEQ ID NO: 6, such as, e.g., amino acids 440-866 of a BoNT/F isoform of SEQ ID NO: 6 or amino acids 440-866 of a BoNT/F subtype of SEQ ID NO: 6. In still another aspect of this embodiment, a BoNT/F translocation domain comprises a non-naturally occurring BoNT/F translocation domain variant, such as, e.g., a conservative BoNT/F translocation domain variant, a non-conservative BoNT/F translocation domain variant, a BoNT/F chimeric translocation domain, an active BoNT/F translocation domain fragment, or any combination thereof. In still another aspect of this embodiment, a BoNT/F translocation domain comprises amino acids 440-866 of a non-naturally occurring BoNT/F translocation domain variant of SEQ ID NO: 6, such as, e.g., amino acids 440-866 of a conservative BoNT/F translocation domain variant of SEQ ID NO: 6, amino acids 440-866 of a non-conservative BoNT/F translocation domain variant of SEQ ID NO: 6, amino acids 440-866 of an active BoNT/F translocation domain fragment of SEQ ID NO: 6, or any combination thereof.

In other aspects of this embodiment, a BoNT/F translocation domain comprises a polypeptide having an amino acid identity of, e.g., at least 70%, at least 75%, at least 80%, at least 85%, at least 90%, at least 95%, or at least 97% to amino acids 440-866 of SEQ ID NO: 6; or at most 70%, at most 75%, at most 80%, at most 85%, at most 90%, at most 95%, or at most 97% to amino acids 440-866 of SEQ ID NO: 6. In yet other aspects of this embodiment, a BoNT/F translocation domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 440-866 of SEQ ID NO: 6; or at most 1, 2, 3, 4, 5, 6, 7, 8, 9, 10, 20, 30, 40, 50, or 100 non-contiguous amino acid deletions, additions, and/or substitutions relative to amino acids 440-866 of SEQ ID NO: 6. In still other aspects of this embodiment, a BoNT/F translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 440-866 of SEQ ID NO: 6; or at most 1, 2, 3, 4, 5, 6, 7, 8, 9, 10, 20, 30, 40, 50, or 100 contiguous amino acid substitutions relative to amino acids 440-866 of SEQ ID NO: 6.

In another embodiment, a Clostridial toxin translocation domain comprises a BoNT/G translocation domain. In an aspect of this embodiment, a BoNT/G translocation domain comprises amino acids 447-865 of SEQ ID NO: 7. In another aspect of this embodiment, a BoNT/G translocation domain comprises a naturally occurring BoNT/G translocation domain variant, such as, e.g., a translocation domain from a BoNT/G isoform or a translocation domain from a BoNT/G subtype. In another aspect of this embodiment, a BoNT/G translocation domain comprises amino acids 447-865 of a naturally occurring BoNT/G translocation domain variant of SEQ ID NO: 7, such as, e.g., amino acids 447-865 of a BoNT/G isoform of SEQ ID NO: 7 or amino acids 447-865 of a BoNT/G subtype of SEQ ID NO: 7. In still another aspect of this embodiment, a BoNT/G translocation domain comprises a non-naturally occurring BoNT/G translocation domain variant, such as, e.g., a conservative BoNT/G translocation domain variant, a non-conservative BoNT/G translocation domain variant, a BoNT/G chimeric translocation domain, an active BoNT/G translocation domain fragment, or any combination thereof. In still another aspect of this embodiment, a BoNT/G translocation domain comprises amino acids 447-865 of a non-naturally occurring BoNT/G translocation domain variant of SEQ ID NO: 7, such as, e.g., amino acids 447-865 of a conservative BoNT/G translocation domain variant of SEQ ID NO: 7, amino acids 447-865 of a non-conservative BoNT/G translocation domain variant of SEQ ID NO: 7, amino acids 447-865 of an active BoNT/G translocation domain fragment of SEQ ID NO: 7, or any combination thereof.

In other aspects of this embodiment, a BoNT/G translocation domain comprises a polypeptide having an amino acid identity of, e.g., at least 70%, at least 75%, at least 80%, at least 85%, at least 90%, at least 95%, or at least 97% to amino acids 447-865 of SEQ ID NO: 7; or at most 70%, at most 75%, at most 80%, at most 85%, at most 90%, at most 95%, or at most 97% to amino acids 447-865 of SEQ ID NO: 7. In yet other aspects of this embodiment, a BoNT/G translocation domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 447-865 of SEQ ID NO: 7; or at most 1, 2, 3, 4, 5, 6, 7, 8, 9, 10, 20, 30, 40, 50, or 100 non-contiguous amino acid deletions, additions, and/or substitutions relative to amino acids 447-865 of SEQ ID NO: 7. In still other aspects of this embodiment, a BoNT/G translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 447-865 of SEQ ID NO: 7; or at most 1, 2, 3, 4, 5, 6, 7, 8, 9, 10, 20, 30, 40, 50, or 100 contiguous amino acid deletions, additions, and/or substitutions relative to amino acids 447-865 of SEQ ID NO: 7.

In another embodiment, a Clostridial toxin translocation domain comprises a TeNT translocation domain. In an aspect of this embodiment, a TeNT translocation domain comprises amino acids 458-881 of SEQ ID NO: 8. In another aspect of this embodiment, a TeNT translocation domain comprises a naturally occurring TeNT translocation domain variant, such as, e.g., a translocation domain from a TeNT isoform or a translocation domain from a TeNT subtype. In another aspect of this embodiment, a TeNT translocation domain comprises amino acids 458-881 of a naturally occurring TeNT translocation domain variant of SEQ ID NO: 8, such as, e.g., amino acids 458-881 of a TeNT isoform of SEQ ID NO: 8 or amino acids 458-881 of a TeNT subtype of SEQ ID NO: 8. In still another aspect of this embodiment, a TeNT translocation domain comprises a non-naturally occurring TeNT translocation domain variant, such as, e.g., a conservative TeNT translocation domain variant, a non-conservative TeNT translocation domain variant, a TeNT chimeric translocation domain, an active TeNT translocation domain fragment, or any combination thereof. In still another aspect of this embodiment, a TeNT translocation domain comprises amino acids 458-881 of a non-naturally occurring TeNT translocation domain variant of SEQ ID NO: 8, such as, e.g., amino acids 458-881 of a conservative TeNT translocation domain variant of SEQ ID NO: 8, amino acids 458-881 of a non-conservative TeNT translocation domain variant of SEQ ID NO: 8, amino acids 458-881 of an active TeNT translocation domain fragment of SEQ ID NO: 8, or any combination thereof.

In other aspects of this embodiment, a TeNT translocation domain comprises a polypeptide having an amino acid identity of, e.g., at least 70%, at least 75%, at least 80%, at least 85%, at least 90%, at least 95%, or at least 97% to amino acids 458-881 of SEQ ID NO: 8; or at most 70%, at most 75%, at most 80%, at most 85%, at most 90%, at most 95%, or at most 97% to amino acids 458-881 of SEQ ID NO: 8. In yet other aspects of this embodiment, a TeNT translocation domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 458-881 of SEQ ID NO: 8; or at most 1, 2, 3, 4, 5, 6, 7, 8, 9, 10, 20, 30, 40, 50, or 100 non-contiguous amino acid deletions, additions, and/or substitutions relative to amino acids 458-881 of SEQ ID NO: 8. In still other aspects of this embodiment, a TeNT translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 458-881 of SEQ ID NO: 8; or at most 1, 2, 3, 4, 5, 6, 7, 8, 9, 10, 20, 30, 40, 50, or 100 contiguous amino acid deletions, additions, and/or substitutions relative to amino acids 458-881 of SEQ ID NO: 8.

In another embodiment, a Clostridial toxin translocation domain comprises a BaNT translocation domain. In an aspect of this embodiment, a BaNT translocation domain comprises amino acids 432-857 of SEQ ID NO: 9. In another aspect of this embodiment, a BaNT translocation domain comprises a naturally occurring BaNT translocation domain variant, such as, e.g., a translocation domain from a BaNT isoform or a translocation domain from a BaNT subtype. In another aspect of this embodiment, a BaNT translocation domain comprises amino acids 432-857 of a naturally occurring BaNT translocation domain variant of SEQ ID NO: 9, such as, e.g., amino acids 432-857 of a BaNT isoform of SEQ ID NO: 9 or amino acids 432-857 of a BaNT subtype of SEQ ID NO: 9. In still another aspect of this embodiment, a BaNT translocation domain comprises a non-naturally occurring BaNT translocation domain variant, such as, e.g., a conservative BaNT translocation domain variant, a non-conservative BaNT translocation domain variant, a BaNT chimeric translocation domain, an active BaNT translocation domain fragment, or any combination thereof. In still another aspect of this embodiment, a BaNT translocation domain comprises amino acids 432-857 of a non-naturally occurring BaNT translocation domain variant of SEQ ID NO: 9, such as, e.g., amino acids 432-857 of a conservative BaNT translocation domain variant of SEQ ID NO: 9, amino acids 432-857 of a non-conservative BaNT translocation domain variant of SEQ ID NO: 9, amino acids 432-857 of an active BaNT translocation domain fragment of SEQ ID NO: 9, or any combination thereof.

In other aspects of this embodiment, a BaNT translocation domain comprises a polypeptide having an amino acid identity of, e.g., at least 70%, at least 75%, at least 80%, at least 85%, at least 90%, at least 95%, or at least 97% to amino acids 432-857 of SEQ ID NO: 9; or at most 70%, at most 75%, at most 80%, at most 85%, at most 90%, at most 95%, or at most 97% to amino acids 432-857 of SEQ ID NO: 9. In yet other aspects of this embodiment, a BaNT translocation domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 432-857 of SEQ ID NO: 9; or at most 1, 2, 3, 4, 5, 6, 7, 8, 9, 10, 20, 30, 40, 50, or 100 non-contiguous amino acid deletions, additions, and/or substitutions relative to amino acids 432-857 of SEQ ID NO: 9. In still other aspects of this embodiment, a BaNT translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 432-857 of SEQ ID NO: 9; or at most 1, 2, 3, 4, 5, 6, 7, 8, 9, 10, 20, 30, 40, 50, or 100 contiguous amino acid deletions, additions, and/or substitutions relative to amino acids 432-857 of SEQ ID NO: 9.

In another embodiment, a Clostridial toxin translocation domain comprises a BuNT translocation domain. In an aspect of this embodiment, a BuNT translocation domain comprises amino acids 423-847 of SEQ ID NO: 10. In another aspect of this embodiment, a BuNT translocation domain comprises a naturally occurring BuNT translocation domain variant, such as, e.g., a translocation domain from a BuNT isoform or a translocation domain from a BuNT subtype. In another aspect of this embodiment, a BuNT translocation domain comprises amino acids 423-847 of a naturally occurring BuNT translocation domain variant of SEQ ID NO: 10, such as, e.g., amino acids 423-847 of a BuNT isoform of SEQ ID NO: 10 or amino acids 423-847 of a BuNT subtype of SEQ ID NO: 10. In still another aspect of this embodiment, a BuNT translocation domain comprises a non-naturally occurring BuNT translocation domain variant, such as, e.g., a conservative BuNT translocation domain variant, a non-conservative BuNT translocation domain variant, a BuNT chimeric translocation domain, an active BuNT translocation domain fragment, or any combination thereof. In still another aspect of this embodiment, a BuNT translocation domain comprises amino acids 423-847 of a non-naturally occurring BuNT translocation domain variant of SEQ ID NO: 10, such as, e.g., amino acids 423-847 of a conservative BuNT translocation domain variant of SEQ ID NO: 10, amino acids 423-847 of a non-conservative BuNT translocation domain variant of SEQ ID NO: 10, amino acids 423-847 of an active BuNT translocation domain fragment of SEQ ID NO: 10, or any combination thereof.

In other aspects of this embodiment, a BuNT translocation domain comprises a polypeptide having an amino acid identity of, e.g., at least 70%, at least 75%, at least 80%, at least 85%, at least 90%, at least 95%, or at least 97% to amino acids 423-847 of SEQ ID NO: 10; or at most 70%, at most 75%, at most 80%, at most 85%, at most 90%, at most 95%, or at most 97% to amino acids 423-847 of SEQ ID NO: 10. In yet other aspects of this embodiment, a BuNT translocation domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 423-847 of SEQ ID NO: 10; or at most 1, 2, 3, 4, 5, 6, 7, 8, 9, 10, 20, 30, 40, 50, or 100 non-contiguous amino acid deletions, additions, and/or substitutions relative to amino acids 423-847 of SEQ ID NO: 10. In still other aspects of this embodiment, a BuNT translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 423-847 of SEQ ID NO: 10; or at most 1, 2, 3, 4, 5, 6, 7, 8, 9, 10, 20, 30, 40, 50, or 100 contiguous amino acid deletions, additions, and/or substitutions relative to amino acids 423-847 of SEQ ID NO: 10.

In another aspect of the invention, a TVEMP comprises, in part, a retargeted peptide binding domain. As used herein, the term “peptide binding domain” refers to an amino acid sequence region able to selectively bind to a cell surface marker characteristic of the target cell under physiological conditions. As used herein, the term “retargeted peptide binding domain” refers to a peptide binding domain that does not selectively bind to a Clostridial toxin receptor under physiological conditions. The cell surface marker may comprise a polypeptide, a polysaccharide, a lipid, a glycoprotein, a lipoprotein, or may have structural characteristics of more than one of these. As used herein, the term “selectively bind” refers to molecule is able to bind its target receptor under physiological conditions, or in vitro conditions substantially approximating physiological conditions, to a statistically significantly greater degree relative to other, non-target receptors.

Thus, in an embodiment, a retargeted binding domain that selectively binds a target receptor has a dissociation equilibrium constant (K_{D}) that is greater for the target receptor relative to a non-target receptor by, e.g., at least one-fold, at least two-fold, at least three-fold, at least four fold, at least five-fold, at least 10 fold, at least 50 fold, at least 100 fold, at least 1000 fold, at least 10,000 fold, or at least 100,000 fold. In another embodiment, a retargeted binding domain that selectively binds a target receptor has a dissociation equilibrium constant (K_{D}) that is greater for the target receptor relative to a non-target receptor by, e.g., about one-fold to about three-fold, about one-fold to about five-fold, about one-fold to about 10-fold, about one-fold to about 100-fold, about one-fold to about 1000-fold, about five-fold to about 10-fold, about five-fold to about 100-fold, about five-fold to about 1000-fold, about 10-fold to about 100-fold, about 10-fold to about 1000-fold, about 10-fold to about 10,000-fold, or about 10-fold to about 1000,00-fold.

An example of a retargeted binding element disclosed in the present specification is an interleukin (IL) peptide binding domain. Non-limiting examples of an IL peptide binding domain include an IL-1, an IL-2, an IL-6, an IL-8, an IL-10, or an IL-11.

Thus, in an embodiment, a retargeted binding domain comprises an IL peptide binding domain. In aspects of this embodiment, an IL peptide binding domain comprises an IL-1, an IL-2, an IL-6, an IL-8, an IL-10, or an IL-11. In other aspects of this embodiment, an IL peptide binding domain comprises SEQ ID NO: 67, SEQ ID NO: 68, SEQ ID NO: 69, SEQ ID NO: 70, SEQ ID NO: 71, or SEQ ID NO: 72. In yet other aspects of this embodiment, an IL peptide binding domain comprises amino acids 123-265 of SEQ ID NO: 67, amino acids 21-153 of SEQ ID NO: 68, amino acids 57-210 of SEQ ID NO: 69, amino acids 21-99 or amino acids 31-94 of SEQ ID NO: 70, amino acids 37-173 or amino acids 19-178 of SEQ ID NO: 71, or amino acids 37-199 of SEQ ID NO: 72.

In other aspects of this embodiment, an IL binding domain comprises a polypeptide having an amino acid identity of, e.g., at least 70%, at least 75%, at least 80%, at least 85%, at least 90%, at least 95%, or at least 97% to SEQ ID NO: 67, SEQ ID NO: 68, SEQ ID NO: 69, SEQ ID NO: 70, SEQ ID NO: 71, or SEQ ID NO: 72; or at most 70%, at most 75%, at most 80%, at most 85%, at most 90%, at most 95%, or at most 97% to SEQ ID NO: 67, SEQ ID NO: 68, SEQ ID NO: 69, SEQ ID NO: 70, SEQ ID NO: 71, or SEQ ID NO: 72. In yet other aspects of this embodiment, an IL binding domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 67, SEQ ID NO: 68, SEQ ID NO: 69, SEQ ID NO: 70, SEQ ID NO: 71, or SEQ ID NO: 72; or at most 1, 2, 3, 4, 5, 6, 7, 8, 9, 10, 15 or 20 non-contiguous amino acid deletions, additions, and/or substitutions relative to SEQ ID NO: 67, SEQ ID NO: 68, SEQ ID NO: 69, SEQ ID NO: 70, SEQ ID NO: 71, or SEQ ID NO: 72. In still other aspects of this embodiment, an IL binding domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 67, SEQ ID NO: 68, SEQ ID NO: 69, SEQ ID NO: 70, SEQ ID NO: 71, or SEQ ID NO: 72; or at most 1, 2, 3, 4, 5, 6, 7, 8, 9, 10, 15 or 20 contiguous amino acid deletions, additions, and/or substitutions relative to SEQ ID NO: 67, SEQ ID NO: 68, SEQ ID NO: 69, SEQ ID NO: 70, SEQ ID NO: 71, or SEQ ID NO: 72.

In other aspects of this embodiment, an IL binding domain comprises a polypeptide having an amino acid identity of, e.g., at least 70%, at least 75%, at least 80%, at least 85%, at least 90%, at least 95%, or at least 97% to amino acids 123-265 of SEQ ID NO: 67, amino acids 21-153 of SEQ ID NO: 68, amino acids 57-210 of SEQ ID NO: 69, amino acids 21-99 or amino acids 31-94 of SEQ ID NO: 70, amino acids 37-173 or amino acids 19-178 of SEQ ID NO: 71, or amino acids 37-199 of SEQ ID NO: 72; or at most 70%, at most 75%, at most 80%, at most 85%, at most 90%, at most 95%, or at most 97% to amino acids 123-265 of SEQ ID NO: 67, amino acids 21-153 of SEQ ID NO: 68, amino acids 57-210 of SEQ ID NO: 69, amino acids 21-99 or amino acids 31-94 of SEQ ID NO: 70, amino acids 37-173 or amino acids 19-178 of SEQ ID NO: 71, or amino acids 37-199 of SEQ ID NO: 72. In yet other aspects of this embodiment, an IL binding domain comprises a polypeptide having, e.g., at least 1, 2, 3, 4, 5, 6, 7, 8, 9, 10, 15 or 20 non-contiguous amino acid deletions, additions, and/or substitutions relative to amino acids 123-265 of SEQ ID NO: 67, amino acids 21-153 of SEQ ID NO: 68, amino acids 57-210 of SEQ ID NO: 69, amino acids 21-99 or amino acids 31-94 of SEQ ID NO: 70, amino acids 37-173 or amino acids 19-178 of SEQ ID NO: 71, or amino acids 37-199 of SEQ ID NO: 72; or at most 1, 2, 3, 4, 5, 6, 7, 8, 9, 10, 15 or 20 non-contiguous amino acid deletions, additions, and/or substitutions relative to amino acids 123-265 of SEQ ID NO: 67, amino acids 21-153 of SEQ ID NO: 68, amino acids 57-210 of SEQ ID NO: 69, amino acids 21-99 or amino acids 31-94 of SEQ ID NO: 70, amino acids 37-173 or amino acids 19-178 of SEQ ID NO: 71, or amino acids 37-199 of SEQ ID NO: 72. In still other aspects of this embodiment, an IL binding domain comprises a polypeptide having, e.g., at least 1, 2, 3, 4, 5, 6, 7, 8, 9, 10, 15 or 20 contiguous amino acid deletions, additions, and/or substitutions relative to amino acids 123-265 of SEQ ID NO: 67, amino acids 21-153 of SEQ ID NO: 68, amino acids 57-210 of SEQ ID NO: 69, amino acids 21-99 or amino acids 31-94 of SEQ ID NO: 70, amino acids 37-173 or amino acids 19-178 of SEQ ID NO: 71, or amino acids 37-199 of SEQ ID NO: 72; or at most 1, 2, 3, 4, 5, 6, 7, 8, 9, 10, 15 or 20 contiguous amino acid deletions, additions, and/or substitutions relative to amino acids 123-265 of SEQ ID NO: 67, amino acids 21-153 of SEQ ID NO: 68, amino acids 57-210 of SEQ ID NO: 69, amino acids 21-99 or amino acids 31-94 of SEQ ID NO: 70, amino acids 37-173 or amino acids 19-178 of SEQ ID NO: 71, or amino acids 37-199 of SEQ ID NO: 72.

Another example of a retargeted binding element disclosed in the present specification is a vascular endothelial growth factor (VEGF) peptide binding domain. Non-limiting examples of a VEGF peptide binding domain include a VEGF-A, a VEGF-B, a VEGF-C, a VEGF-D, or a placenta growth factor (PIGF).

Thus, in an embodiment, a retargeted binding element comprises a VEGF peptide binding domain. In aspects of this embodiment, a VEGF peptide binding domain comprises a VEGF-A, a VEGF-B, a VEGF-C, a VEGF-D, or a PIGF. In aspects of this embodiment, a VEGF peptide binding domain comprises SEQ ID NO: 73, SEQ ID NO: 74, SEQ ID NO: 75, SEQ ID NO: 76, SEQ ID NO: 77, or SEQ ID NO: 78. In other aspects of this embodiment, a VEGF peptide binding domain comprises amino acids 50-133 of SEQ ID NO: 73, amino acids 45-127 of SEQ ID NO: 74, amino acids 129-214 of SEQ ID NO: 75, amino acids 109-194 of SEQ ID NO: 76, amino acids 46-163, amino acids 49-162, amino acids 168-345, amino acids 244-306, or amino acids 248-340 of SEQ ID NO: 77, or amino acids 50-131 or amino acids 132-203 of SEQ ID NO: 78.

In other aspects of this embodiment, a VEGF peptide binding domain comprises a polypeptide having an amino acid identity of, e.g., at least 70%, at least 75%, at least 80%, at least 85%, at least 90%, at least 95%, or at least 97% to SEQ ID NO: 73, SEQ ID NO: 74, SEQ ID NO: 75, SEQ ID NO: 76, SEQ ID NO: 77, or SEQ ID NO: 78; or at most 70%, at most 75%, at most 80%, at most 85%, at most 90%, at most 95%, or at most 97% to SEQ ID NO: 73, SEQ ID NO: 74, SEQ ID NO: 75, SEQ ID NO: 76, SEQ ID NO: 77, or SEQ ID NO: 78. In yet other aspects of this embodiment, a VEGF peptide binding domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 73, SEQ ID NO: 74, SEQ ID NO: 75, SEQ ID NO: 76, SEQ ID NO: 77, or SEQ ID NO: 78; or at most 1, 2, 3, 4, 5, 6, 7, 8, 9, 10, 15 or 20 non-contiguous amino acid deletions, additions, and/or substitutions relative to SEQ ID NO: 73, SEQ ID NO: 74, SEQ ID NO: 75, SEQ ID NO: 76, SEQ ID NO: 77, or SEQ ID NO: 78. In still other aspects of this embodiment, a VEGF peptide binding domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 73, SEQ ID NO: 74, SEQ ID NO: 75, SEQ ID NO: 76, SEQ ID NO: 77, or SEQ ID NO: 78; or at most 1, 2, 3, 4, 5, 6, 7, 8, 9, 10, 15 or 20 contiguous amino acid deletions, additions, and/or substitutions relative to SEQ ID NO: 73, SEQ ID NO: 74, SEQ ID NO: 75, SEQ ID NO: 76, SEQ ID NO: 77, or SEQ ID NO: 78.

In other aspects of this embodiment, a VEGF peptide binding domain comprises a polypeptide having an amino acid identity of, e.g., at least 70%, at least 75%, at least 80%, at least 85%, at least 90%, at least 95%, or at least 97% to amino acids 50-133 of SEQ ID NO: 73, amino acids 45-127 of SEQ ID NO: 74, amino acids 129-214 of SEQ ID NO: 75, amino acids 109-194 of SEQ ID NO: 76, amino acids 46-163, amino acids 49-162, amino acids 168-345, amino acids 244-306, or amino acids 248-340 of SEQ ID NO: 77, or amino acids 50-131 or amino acids 132-203 of SEQ ID NO: 78; or at most 70%, at most 75%, at most 80%, at most 85%, at most 90%, at most 95%, or at most 97% to amino acids 50-133 of SEQ ID NO: 73, amino acids 45-127 of SEQ ID NO: 74, amino acids 129-214 of SEQ ID NO: 75, amino acids 109-194 of SEQ ID NO: 76, amino acids 46-163, amino acids 49-162, amino acids 168-345, amino acids 244-306, or amino acids 248-340 of SEQ ID NO: 77, or amino acids 50-131 or amino acids 132-203 of SEQ ID NO: 78. In yet other aspects of this embodiment, a VEGF peptide binding domain comprises a polypeptide having, e.g., at least 1, 2, 3, 4, 5, 6, 7, 8, 9, 10, 15 or 20 non-contiguous amino acid deletions, additions, and/or substitutions relative to amino acids 50-133 of SEQ ID NO: 73, amino acids 45-127 of SEQ ID NO: 74, amino acids 129-214 of SEQ ID NO: 75, amino acids 109-194 of SEQ ID NO: 76, amino acids 46-163, amino acids 49-162, amino acids 168-345, amino acids 244-306, or amino acids 248-340 of SEQ ID NO: 77, or amino acids 50-131 or amino acids 132-203 of SEQ ID NO: 78; or at most 1, 2, 3, 4, 5, 6, 7, 8, 9, 10, 15 or 20 non-contiguous amino acid deletions, additions, and/or substitutions relative to amino acids 50-133 of SEQ ID NO: 73, amino acids 45-127 of SEQ ID NO: 74, amino acids 129-214 of SEQ ID NO: 75, amino acids 109-194 of SEQ ID NO: 76, amino acids 46-163, amino acids 49-162, amino acids 168-345, amino acids 244-306, or amino acids 248-340 of SEQ ID NO: 77, or amino acids 50-131 or amino acids 132-203 of SEQ ID NO: 78. In still other aspects of this embodiment, a VEGF peptide binding domain comprises a polypeptide having, e.g., at least 1, 2, 3, 4, 5, 6, 7, 8, 9, 10, 15 or 20 contiguous amino acid deletions, additions, and/or substitutions relative to amino acids 50-133 of SEQ ID NO: 73, amino acids 45-127 of SEQ ID NO: 74, amino acids 129-214 of SEQ ID NO: 75, amino acids 109-194 of SEQ ID NO: 76, amino acids 46-163, amino acids 49-162, amino acids 168-345, amino acids 244-306, or amino acids 248-340 of SEQ ID NO: 77, or amino acids 50-131 or amino acids 132-203 of SEQ ID NO: 78; or at most 1, 2, 3, 4, 5, 6, 7, 8, 9, 10, 15 or 20 contiguous amino acid deletions, additions, and/or substitutions relative to amino acids 50-133 of SEQ ID NO: 73, amino acids 45-127 of SEQ ID NO: 74, amino acids 129-214 of SEQ ID NO: 75, amino acids 109-194 of SEQ ID NO: 76, amino acids 46-163, amino acids 49-162, amino acids 168-345, amino acids 244-306, or amino acids 248-340 of SEQ ID NO: 77, or amino acids 50-131 or amino acids 132-203 of SEQ ID NO: 78.

Another example of a retargeted binding element disclosed in the present specification is an insulin-like growth factor (IGF) peptide binding domain. Non-limiting examples of an IGF peptide binding domain include an IGF-1 or an IGF-2.

Thus, in an embodiment, a retargeted binding element comprises an IGF peptide binding domain. In aspects of this embodiment, an IGF peptide binding domain comprises an IGF-1 or an IGF-2. In aspects of this embodiment, an IGF peptide binding domain comprises SEQ ID NO: 79 or SEQ ID NO: 80. In other aspects of this embodiment, an IGF peptide binding domain comprises amino acids 52-109 or amino acids 49-118 of SEQ ID NO: 79, or amino acids 31-84 or amino acids 25-180 of SEQ ID NO: 80.

In other aspects of this embodiment, an IGF peptide binding domain comprises a polypeptide having an amino acid identity of, e.g., at least 70%, at least 75%, at least 80%, at least 85%, at least 90%, at least 95%, or at least 97% to SEQ ID NO: 79 or SEQ ID NO: 80; or at most 70%, at most 75%, at most 80%, at most 85%, at most 90%, at most 95%, or at most 97% to SEQ ID NO: 79 or SEQ ID NO: 80. In yet other aspects of this embodiment, an IGF peptide binding domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 79 or SEQ ID NO: 80; or at most 1, 2, 3, 4, 5, 6, 7, 8, 9, 10, 15 or 20 non-contiguous amino acid deletions, additions, and/or substitutions relative to SEQ ID NO: 79 or SEQ ID NO: 80. In still other aspects of this embodiment, an IGF peptide binding domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 79 or SEQ ID NO: 80; or at most 1, 2, 3, 4, 5, 6, 7, 8, 9, 10, 15 or 20 contiguous amino acid deletions, additions, and/or substitutions relative to SEQ ID NO: 79 or SEQ ID NO: 80.

In other aspects of this embodiment, an IGF peptide binding domain comprises a polypeptide having an amino acid identity of, e.g., at least 70%, at least 75%, at least 80%, at least 85%, at least 90%, at least 95%, or at least 97% to amino acids 52-109 or amino acids 49-118 of SEQ ID NO: 79, or amino acids 31-84 or amino acids 25-180 of SEQ ID NO: 80; or at most 70%, at most 75%, at most 80%, at most 85%, at most 90%, at most 95%, or at most 97% to amino acids 52-109 or amino acids 49-118 of SEQ ID NO: 79, or amino acids 31-84 or amino acids 25-180 of SEQ ID NO: 80. In yet other aspects of this embodiment, an IGF peptide binding domain comprises a polypeptide having, e.g., at least 1, 2, 3, 4, 5, 6, 7, 8, 9, 10, 15 or 20 non-contiguous amino acid deletions, additions, and/or substitutions relative to amino acids 52-109 or amino acids 49-118 of SEQ ID NO: 79, or amino acids 31-84 or amino acids 25-180 of SEQ ID NO: 80; or at most 1, 2, 3, 4, 5, 6, 7, 8, 9, 10, 15 or 20 non-contiguous amino acid deletions, additions, and/or substitutions relative to amino acids 52-109 or amino acids 49-118 of SEQ ID NO: 79, or amino acids 31-84 or amino acids 25-180 of SEQ ID NO: 80. In still other aspects of this embodiment, an IGF peptide binding domain comprises a polypeptide having, e.g., at least 1, 2, 3, 4, 5, 6, 7, 8, 9, 10, 15 or 20 contiguous amino acid deletions, additions, and/or substitutions relative to amino acids 52-109 or amino acids 49-118 of SEQ ID NO: 79, or amino acids 31-84 or amino acids 25-180 of SEQ ID NO: 80; or at most 1, 2, 3, 4, 5, 6, 7, 8, 9, 10, 15 or 20 contiguous amino acid deletions, additions, and/or substitutions relative to amino acids 52-109 or amino acids 49-118 of SEQ ID NO: 79, or amino acids 31-84 or amino acids 25-180 of SEQ ID NO: 80.

Another example of a retargeted binding element disclosed in the present specification is an epidermal growth factor (EGF) peptide binding domain. Non-limiting examples of an EGF peptide binding domain include an EGF, a heparin-binding EGF-like growth factor (HB-EGF), a transforming growth factor-α (TGF-α), an amphiregulin (AR), an epiregulin (EPR), an epigen (EPG), a betacellulin (BTC), a neuregulin-1 (NRG1), a neuregulin-2 (NRG2), a neuregulin-3, (NRG3), or a neuregulin-4 (NRG4).

Thus, in an embodiment, a retargeted binding element comprises an EGF peptide binding domain. In aspects of this embodiment, an EGF peptide binding domain comprises an EGF, a HB-EGF, a TGF-α, an AR, an EPR, an EPG, a BTC, a NRG-1, a NRG-2, a NRG-3, or a NRG-4. In aspects of this embodiment, an EGF peptide binding domain comprises SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91. In other aspects of this embodiment, an EGF peptide binding domain comprises amino acids 101-251 or amino acids 107-251 of SEQ ID NO: 84, amino acids 63-108 of SEQ ID NO: 85, amino acids 23-154 of SEQ ID NO: 86, amino acids 235-630 of SEQ ID NO: 88, amino acids 398-718 of SEQ ID NO: 89, or amino acids 353-648 of SEQ ID NO: 90. In yet another aspect of this embodiment, an EGF peptide binding domain comprises a NRG-2 isoform like a NRG-2 isoform 1, a NRG-2 isoform 2, a NRG-2 isoform 3, a NRG-2 isoform 4, a NRG-2 isoform 5, or a NRG-2 isoform 6.

In other aspects of this embodiment, an EGF peptide binding domain comprises a polypeptide having an amino acid identity of, e.g., at least 70%, at least 75%, at least 80%, at least 85%, at least 90%, at least 95%, or at least 97% to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91; or at most 70%, at most 75%, at most 80%, at most 85%, at most 90%, at most 95%, or at most 97% to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91. In yet other aspects of this embodiment, an EGF peptide binding domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91; or at most 1, 2, 3, 4, 5, 6, 7, 8, 9, 10, 15 or 20 non-contiguous amino acid deletions, additions, and/or substitutions relative to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91. In still other aspects of this embodiment, an EGF peptide binding domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91; or at most 1, 2, 3, 4, 5, 6, 7, 8, 9, 10, 15 or 20 contiguous amino acid deletions, additions, and/or substitutions relative to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91.

In other aspects of this embodiment, an EGF peptide binding domain comprises a polypeptide having an amino acid identity of, e.g., at least 70%, at least 75%, at least 80%, at least 85%, at least 90%, at least 95%, or at least 97% to amino acids 101-251 or amino acids 107-251 of SEQ ID NO: 84, amino acids 63-108 of SEQ ID NO: 85, amino acids 23-154 of SEQ ID NO: 86, amino acids 235-630 of SEQ ID NO: 88, amino acids 398-718 of SEQ ID NO: 89, or amino acids 353-648 of SEQ ID NO: 90; or at most 70%, at most 75%, at most 80%, at most 85%, at most 90%, at most 95%, or at most 97% to amino acids 101-251 or amino acids 107-251 of SEQ ID NO: 84, amino acids 63-108 of SEQ ID NO: 85, amino acids 23-154 of SEQ ID NO: 86, amino acids 235-630 of SEQ ID NO: 88, amino acids 398-718 of SEQ ID NO: 89, or amino acids 353-648 of SEQ ID NO: 90. In yet other aspects of this embodiment, an EGF peptide binding domain comprises a polypeptide having, e.g., at least 1, 2, 3, 4, 5, 6, 7, 8, 9, 10, 15 or 20 non-contiguous amino acid deletions, additions, and/or substitutions relative to amino acids 101-251 or amino acids 107-251 of SEQ ID NO: 84, amino acids 63-108 of SEQ ID NO: 85, amino acids 23-154 of SEQ ID NO: 86, amino acids 235-630 of SEQ ID NO: 88, amino acids 398-718 of SEQ ID NO: 89, or amino acids 353-648 of SEQ ID NO: 90; or at most 1, 2, 3, 4, 5, 6, 7, 8, 9, 10, 15 or 20 non-contiguous amino acid deletions, additions, and/or substitutions relative to amino acids 101-251 or amino acids 107-251 of SEQ ID NO: 84, amino acids 63-108 of SEQ ID NO: 85, amino acids 23-154 of SEQ ID NO: 86, amino acids 235-630 of SEQ ID NO: 88, amino acids 398-718 of SEQ ID NO: 89, or amino acids 353-648 of SEQ ID NO: 90. In still other aspects of this embodiment, an EGF peptide binding domain comprises a polypeptide having, e.g., at least 1, 2, 3, 4, 5, 6, 7, 8, 9, 10, 15 or 20 contiguous amino acid deletions, additions, and/or substitutions relative to amino acids 101-251 or amino acids 107-251 of SEQ ID NO: 84, amino acids 63-108 of SEQ ID NO: 85, amino acids 23-154 of SEQ ID NO: 86, amino acids 235-630 of SEQ ID NO: 88, amino acids 398-718 of SEQ ID NO: 89, or amino acids 353-648 of SEQ ID NO: 90; or at most 1, 2, 3, 4, 5, 6, 7, 8, 9, 10, 15 or 20 contiguous amino acid deletions, additions, and/or substitutions relative to amino acids 101-251 or amino acids 107-251 of SEQ ID NO: 84, amino acids 63-108 of SEQ ID NO: 85, amino acids 23-154 of SEQ ID NO: 86, amino acids 235-630 of SEQ ID NO: 88, amino acids 398-718 of SEQ ID NO: 89, or amino acids 353-648 of SEQ ID NO: 90.

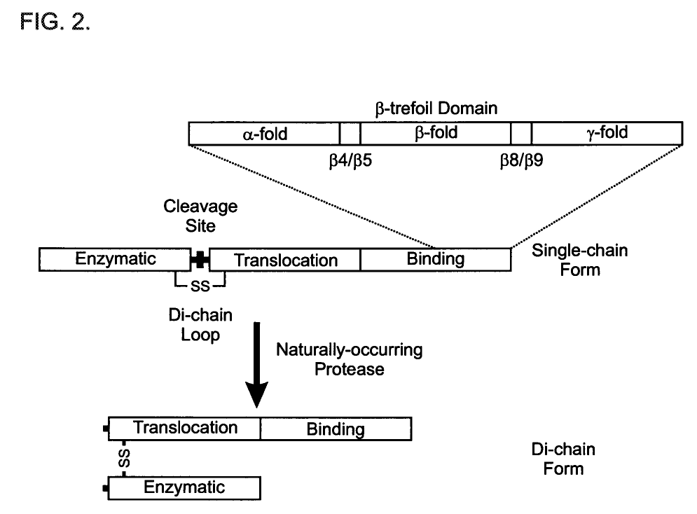

Clostridial toxins are each translated as a single-chain polypeptide of approximately 150 kDa that is subsequently cleaved by proteolytic scission within a disulfide loop by a naturally-occurring protease. This cleavage occurs within the discrete di-chain loop region created between two cysteine residues that form a disulfide bridge. This posttranslational processing yields a di-chain molecule comprising an approximately 50 kDa light chain (LC) and an approximately 100 kDa heavy chain (HC) held together by the single disulfide bond and non-covalent interactions between the two chains (FIG. 2). To facilitate recombinant production of a TVEMP, an exogenous protease cleavage site can be used to convert the single-chain polypeptide form of a TVEMP disclosed in the present specification into the di-chain form. See, e.g., Steward, L. E. et al., Modified Clostridial Toxins with Enhanced Targeting Capabilities For Endogenous Clostridial Toxin Receptor Systems, U.S. Patent Publication No. US 2008/0096248 (Apr. 24, 2008); Steward, L. E. et al., Activatable Clostridial Toxins, U.S. Patent Publication No. US 2008/0032930 (Feb. 7, 2008); Steward, supra, (2007); Dolly, supra, (2007); Foster, supra, WO 2006/059093 (2006); and Foster, supra, WO 2006/059105 (2006), each of which is hereby incorporated by reference in its entirety.

It is envisioned that any and all protease cleavage sites can be used to convert the single-chain polypeptide form of a Clostridial toxin into the di-chain form, including, without limitation, endogenous di-chain loop protease cleavage sites and exogenous protease cleavage sites. Thus, in an aspect of the invention, a TVEMP comprises, in part, an endogenous protease cleavage site within a di-chain loop region. In another aspect of the invention, a TVEMP comprises, in part, an exogenous protease cleavage site within a di-chain loop region. As used herein, the term “di-chain loop region” refers to the amino acid sequence of a Clostridial toxin containing a protease cleavage site used to convert the single-chain form of a Clostridial toxin into the di-chain form. Non-limiting examples of a Clostridial toxin di-chain loop region, include, a di-chain loop region of BoNT/A comprising amino acids 430-454 of SEQ ID NO: 1; a di-chain loop region of BoNT/B comprising amino acids 437-446 of SEQ ID NO: 2; a di-chain loop region of BoNT/C1 comprising amino acids 437-453 of SEQ ID NO: 3; a di-chain loop region of BoNT/D comprising amino acids 437-450 of SEQ ID NO: 4; a di-chain loop region of BoNT/E comprising amino acids 412-426 of SEQ ID NO: 5; a di-chain loop region of BoNT/F comprising amino acids 429-445 of SEQ ID NO: 6; a di-chain loop region of BoNT/G comprising amino acids 436-450 of SEQ ID NO: 7; a di-chain loop region of TeNT comprising amino acids 439-467 of SEQ ID NO: 8; a di-chain loop region of TeNT comprising amino acids 421-435 of SEQ ID NO: 9; and a di-chain loop region of TeNT comprising amino acids 412-426 of SEQ ID NO: 10 (Table 4).

TABLE 4
Di-chain Loop Region

| | Di-chain Loop Region Containing |
|---|---|
| | the Naturally-occurring Protease |
| Toxin | Cleavage Site |
| BoNT/A | CVRGIITSKTKSLDKGYNK*----ALNDLC |
| BoNT/B | CKSVK*-------------------APGIC |
| BoNT/C1 | CHKAIDGRSLYNK*------------TLDC |
| BoNT/D | CLRLTKNSR*---------------DDSTC |
| BoNT/E | CKNIVSVKGIR*--------------KSIC |
| BoNT/F | CKSVIPRKGTK*------------APPRLC |
| BoNT/G | CKPVMYKNTGK*--------------SEQC |
| TeNT | CKKIIPPTNIRENLYNRTA*SLTDLGGELC |
| BaNT | CKS-IVSKKGTK*------------NSLC |
| BuNT | CKN-IVSVKGIR*--------------KSIC |

The amino acid sequence displayed are as follows: BoNT/A, residues 430-454 of SEQ ID NO: 1; BoNT/B, residues 437-446 of SEQ ID NO: 2; BoNT/C1, residues 437-453 of SEQ ID NO: 3; BoNT/D, residues 437-450 of SEQ ID NO: 4; BoNT/E, residues 412-426 of SEQ ID NO: 5; BoNT/F, residues 429-445 of SEQ ID NO: 6; BoNT/G, residues 436-450 of SEQ ID NO: 7; TeNT, residues 439-467 of SEQ ID NO: 8; BaNT, residues 421-435 of SEQ ID NO: 9; and BuNT, residues 412-426 of SEQ ID NO: 10. An asterisks (*) indicates the peptide bond that is cleaved by a Clostridial toxin protease.

As used herein, the term “endogenous di-chain loop protease cleavage site” is synonymous with a “naturally occurring di-chain loop protease cleavage site” and refers to a naturally occurring protease cleavage site found within the di-chain loop region of a naturally occurring Clostridial toxin and includes, without limitation, naturally occurring Clostridial toxin di-chain loop protease cleavage site variants, such as, e.g., Clostridial toxin di-chain loop protease cleavage site isoforms and Clostridial toxin di-chain loop protease cleavage site subtypes. Non-limiting examples of an endogenous protease cleavage site, include, e.g., a BoNT/A di-chain loop protease cleavage site, a BoNT/B di-chain loop protease cleavage site, a BoNT/C1 di-chain loop protease cleavage site, a BoNT/D di-chain loop protease cleavage site, a BoNT/E di-chain loop protease cleavage site, a BoNT/F di-chain loop protease cleavage site, a BoNT/G di-chain loop protease cleavage site and a TeNT di-chain loop protease cleavage site.

As mentioned above, Clostridial toxins are translated as a single-chain polypeptide of approximately 150 kDa that is subsequently cleaved by proteolytic scission within a disulfide loop by a naturally-occurring protease. This posttranslational processing yields a di-chain molecule comprising an approximately 50 kDa light chain (LC) and an approximately 100 kDa heavy chain (HC) held together by a single disulphide bond and noncovalent interactions. While the identity of the protease is currently unknown, the di-chain loop protease cleavage site for many Clostridial toxins has been determined. In BoNTs, cleavage at K448-A449 converts the single polypeptide form of BoNT/A into the di-chain form; cleavage at K441-A442 converts the single polypeptide form of BoNT/B into the di-chain form; cleavage at K449-T450 converts the single polypeptide form of BoNT/C1 into the di-chain form; cleavage at R445-D446 converts the single polypeptide form of BoNT/D into the di-chain form; cleavage at R422-K423 converts the single polypeptide form of BoNT/E into the di-chain form; cleavage at K439-A440 converts the single polypeptide form of BoNT/F into the di-chain form; and cleavage at K446-S447 converts the single polypeptide form of BoNT/G into the di-chain form. Proteolytic cleavage of the single polypeptide form of TeNT at A457-S458 results in the di-chain form. Proteolytic cleavage of the single polypeptide form of BaNT at K431-N432 results in the di-chain form. Proteolytic cleavage of the single polypeptide form of BuNT at R422-K423 results in the di-chain form. Such a di-chain loop protease cleavage site is operably-linked in-frame to a TVEMP as a fusion protein. However, it should also be noted that additional cleavage sites within the di-chain loop also appear to be cleaved resulting in the generation of a small peptide fragment being lost. As a non-limiting example, BoNT/A single-chain polypeptide cleavage ultimately results in the loss of a ten amino acid fragment within the di-chain loop.

Thus, in an embodiment, a protease cleavage site comprising an endogenous Clostridial toxin di-chain loop protease cleavage site is used to convert the single-chain toxin into the di-chain form. In aspects of this embodiment, conversion into the di-chain form by proteolytic cleavage occurs from a site comprising, e.g., a BoNT/A di-chain loop protease cleavage site, a BoNT/B di-chain loop protease cleavage site, a BoNT/C1 di-chain loop protease cleavage site, a BoNT/D di-chain loop protease cleavage site, a BoNT/E di-chain loop protease cleavage site, a BoNT/F di-chain loop protease cleavage site, a BoNT/G di-chain loop protease cleavage site, a TeNT di-chain loop protease cleavage site, a BaNT di-chain loop protease cleavage site, or a BuNT di-chain loop protease cleavage site.

In other aspects of this embodiment, conversion into the di-chain form by proteolytic cleavage occurs from a site comprising, e.g., a di-chain loop region of BoNT/A comprising amino acids 430-454 of SEQ ID NO: 1; a di-chain loop region of BoNT/B comprising amino acids 437-446 of SEQ ID NO: 2; a di-chain loop region of BoNT/C1 comprising amino acids 437-453 of SEQ ID NO: 3; a di-chain loop region of BoNT/D comprising amino acids 437-450 of SEQ ID NO: 4; a di-chain loop region of BoNT/E comprising amino acids 412-426 of SEQ ID NO: 5; a di-chain loop region of BoNT/F comprising amino acids 429-445 of SEQ ID NO: 6; a di-chain loop region of BoNT/G comprising amino acids 436-450 of SEQ ID NO: 7; or a di-chain loop region of TeNT comprising amino acids 439-467 of SEQ ID NO: 8; a di-chain loop region of BaNT comprising amino acids 421-435 of SEQ ID NO: 9; or a di-chain loop region of BuNT comprising amino acids 412-426 of SEQ ID NO: 10.

It is also envisioned that an exogenous protease cleavage site can be used to convert the single-chain polypeptide form of a TVEMP disclosed in the present specification into the di-chain form. As used herein, the term “exogenous protease cleavage site” is synonymous with a “non-naturally occurring protease cleavage site” or “non-native protease cleavage site” and refers to a protease cleavage site that is not normally present in a di-chain loop region from a naturally occurring Clostridial toxin, with the proviso that the exogenous protease cleavage site is not a human protease cleavage site or a protease cleavage site that is susceptible to a protease being expressed in the host cell that is expressing a construct encoding an activatable polypeptide disclosed in the present specification. It is envisioned that any and all exogenous protease cleavage sites can be used to convert the single-chain polypeptide form of a Clostridial toxin into the di-chain form are useful to practice aspects of the present invention. Non-limiting examples of exogenous protease cleavage sites include, e.g., a plant papain cleavage site, an insect papain cleavage site, a crustacian papain cleavage site, an enterokinase cleavage site, a human rhinovirus 3C protease cleavage site, a human enterovirus 3C protease cleavage site, a tobacco etch virus (TEV) protease cleavage site, a Tobacco Vein Mottling Virus (TVMV) cleavage site, a subtilisin cleavage site, a hydroxylamine cleavage site, or a Caspase 3 cleavage site.

It is envisioned that an exogenous protease cleavage site of any and all lengths can be useful in aspects of the present invention with the proviso that the exogenous protease cleavage site is capable of being cleaved by its respective protease. Thus, in aspects of this embodiment, an exogenous protease cleavage site can have a length of, e.g., at least 6, 7, 8, 9, 10, 15, 20, 25, 30, 40, 50, or at least 60 amino acids; or at most 6, 7, 8, 9, 10, 15, 20, 25, 30, 40, 50, or at least 60 amino acids.

In an embodiment, an exogenous protease cleavage site is located within the di-chain loop of a TVEMP. In aspects of this embodiment, a TVEMP comprises an exogenous protease cleavage site comprises, e.g., a plant papain cleavage site, an insect papain cleavage site, a crustacian papain cleavage site, a non-human enterokinase protease cleavage site, a Tobacco Etch Virus protease cleavage site, a Tobacco Vein Mottling Virus protease cleavage site, a human rhinovirus 3C protease cleavage site, a human enterovirus 3C protease cleavage site, a subtilisin cleavage site, a hydroxylamine cleavage site, a SUMO/ULP-1 protease cleavage site, and a non-human Caspase 3 cleavage site. In other aspects of this embodiment, an exogenous protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In an aspect of this embodiment, an exogenous protease cleavage site can comprise, e.g., a non-human enterokinase cleavage site is located within the di-chain loop of a TVEMP. In other aspects of the embodiment, an exogenous protease cleavage site can comprise, e.g., a bovine enterokinase protease cleavage site located within the di-chain loop of a TVEMP. In other aspects of the embodiment, an exogenous protease cleavage site can comprise, e.g., a bovine enterokinase protease cleavage site located within the di-chain loop of a TVEMP comprises SEQ ID NO: 21. In still other aspects of this embodiment, a bovine enterokinase protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In another aspect of this embodiment, an exogenous protease cleavage site can comprise, e.g., a Tobacco Etch Virus protease cleavage site is located within the di-chain loop of a TVEMP. In other aspects of the embodiment, an exogenous protease cleavage site can comprise, e.g., a Tobacco Etch Virus protease cleavage site located within the di-chain loop of a TVEMP comprises the consensus sequence E-P5-P4-Y-P2-Q*-G (SEQ ID NO: 22) or E-P5-P4-Y-P2-Q*-S (SEQ ID NO: 23), where P2, P4 and P5 can be any amino acid. In other aspects of the embodiment, an exogenous protease cleavage site can comprise, e.g., a Tobacco Etch Virus protease cleavage site located within the di-chain loop of a TVEMP comprises SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 or SEQ ID NO: 33. In still other aspects of this embodiment, a Tobacco Etch Virus protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In another aspect of this embodiment, an exogenous protease cleavage site can comprise, e.g., a Tobacco Vein Mottling Virus protease cleavage site is located within the di-chain loop of a TVEMP. In other aspects of the embodiment, an exogenous protease cleavage site can comprise, e.g., a Tobacco Vein Mottling Virus protease cleavage site located within the di-chain loop of a TVEMP comprises the consensus sequence P6-P5-V-R-F-Q*-G (SEQ ID NO: 34) or P6-P5-V-R-F-Q*-S (SEQ ID NO: 35), where P5 and P6 can be any amino acid. In other aspects of the embodiment, an exogenous protease cleavage site can comprise, e.g., a Tobacco Vein Mottling Virus protease cleavage site located within the di-chain loop of a TVEMP comprises SEQ ID NO: 36, SEQ ID NO: 37, SEQ ID NO: 38, or SEQ ID NO: 39. In still other aspects of this embodiment, a Tobacco Vein Mottling Virus protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In still another aspect of this embodiment, an exogenous protease cleavage site can comprise, e.g., a human rhinovirus 3C protease cleavage site is located within the di-chain loop of a TVEMP. In other aspects of the embodiment, an exogenous protease cleavage site can comprise, e.g., a human rhinovirus 3C protease cleavage site located within the di-chain loop of a TVEMP comprises the consensus sequence P5-P4-L-F-Q*-G-P (SEQ ID NO: 40), where P4 is G, A, V, L, I, M, S or T and P5 can any amino acid, with D or E preferred. In other aspects of the embodiment, an exogenous protease cleavage site can comprise, e.g., a human rhinovirus 3C protease cleavage site located within the di-chain loop of a TVEMP comprises SEQ ID NO: 41, SEQ ID NO: 42, SEQ ID NO: 43, SEQ ID NO: 44, SEQ ID NO: 45 or SEQ ID NO: 46. In other aspects of the embodiment, an exogenous protease cleavage site can comprise, e.g., a human rhinovirus 3C protease located within the di-chain loop of a TVEMP that can be cleaved by PRESCISSION®, a modified human rhinovirus 3C protease (GE Healthcare Biosciences, Piscataway, N.J.). In still other aspects of this embodiment, a human rhinovirus 3C protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In yet another aspect of this embodiment, an exogenous protease cleavage site can comprise, e.g., a subtilisin cleavage site is located within the di-chain loop of a TVEMP. In other aspects of the embodiment, an exogenous protease cleavage site can comprise, e.g., a subtilisin cleavage site located within the di-chain loop of a TVEMP comprises the consensus sequence P6-P5-P4-P3-H*-Y (SEQ ID NO: 47) or P6-P5-P4-P3-Y-H* (SEQ ID NO: 48), where P3, P4 and P5 and P6 can be any amino acid. In other aspects of the embodiment, an exogenous protease cleavage site can comprise, e.g., a subtilisin cleavage site located within the di-chain loop of a TVEMP comprises SEQ ID NO: 49, SEQ ID NO: 50, or SEQ ID NO: 51. In other aspects of the embodiment, an exogenous protease cleavage site can comprise, e.g., a subtilisin cleavage site located within the di-chain loop of a TVEMP that can be cleaved by GENENASE®, a modified subtilisin (New England Biolabs, Ipswich, Mass.). In still other aspects of this embodiment, a subtilisin cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In yet another aspect of this embodiment, an exogenous protease cleavage site can comprise, e.g., a hydroxylamine cleavage site is located within the di-chain loop of a TVEMP. In other aspects of the embodiment, an exogenous protease cleavage site can comprise, e.g., a hydroxylamine cleavage site comprising multiples of the dipeptide N*G. In other aspects of the embodiment, an exogenous protease cleavage site can comprise, e.g., a hydroxylamine cleavage site located within the di-chain loop of a TVEMP comprises SEQ ID NO: 52, or SEQ ID NO: 53. In still other aspects of this embodiment, a hydroxylamine cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In yet another aspect of this embodiment, an exogenous protease cleavage site can comprise, e.g., a SUMO/ULP-1 protease cleavage site is located within the di-chain loop of a TVEMP. In other aspects of the embodiment, an exogenous protease cleavage site can comprise, e.g., a SUMO/ULP-1 protease cleavage site located within the di-chain loop of a TVEMP comprising the consensus sequence G-G*-P1′-P2′-P3′ (SEQ ID NO: 54), where P1′, P2′, and P3′ can be any amino acid. In other aspects of the embodiment, an exogenous protease cleavage site can comprise, e.g., a SUMO/ULP-1 protease cleavage site located within the di-chain loop of a TVEMP comprises SEQ ID NO: 55. In still other aspects of this embodiment, a SUMO/ULP-1 protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In an aspect of this embodiment, an exogenous protease cleavage site can comprise, e.g., a non-human Caspase 3 cleavage site is located within the di-chain loop of a TVEMP. In other aspects of the embodiment, an exogenous protease cleavage site can comprise, e.g., a mouse Caspase 3 protease cleavage site located within the di-chain loop of a TVEMP. In other aspects of the embodiment, an exogenous protease cleavage site can comprise, e.g., a non-human Caspase 3 protease cleavage site located within the di-chain loop of a TVEMP comprises the consensus sequence D-P3-P2-D*P1′ (SEQ ID NO: 56), where P3 can be any amino acid, with E preferred, P2 can be any amino acid and P1′ can any amino acid, with G or S preferred. In other aspects of the embodiment, an exogenous protease cleavage site can comprise, e.g., a non-human Caspase 3 protease cleavage site located within the di-chain loop of a TVEMP comprising SEQ ID NO: 57, SEQ ID NO: 58, SEQ ID NO: 59, SEQ ID NO: 60, SEQ ID NO: 61, or SEQ ID NO: 62. In still other aspects of this embodiment, a bovine enterokinase protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

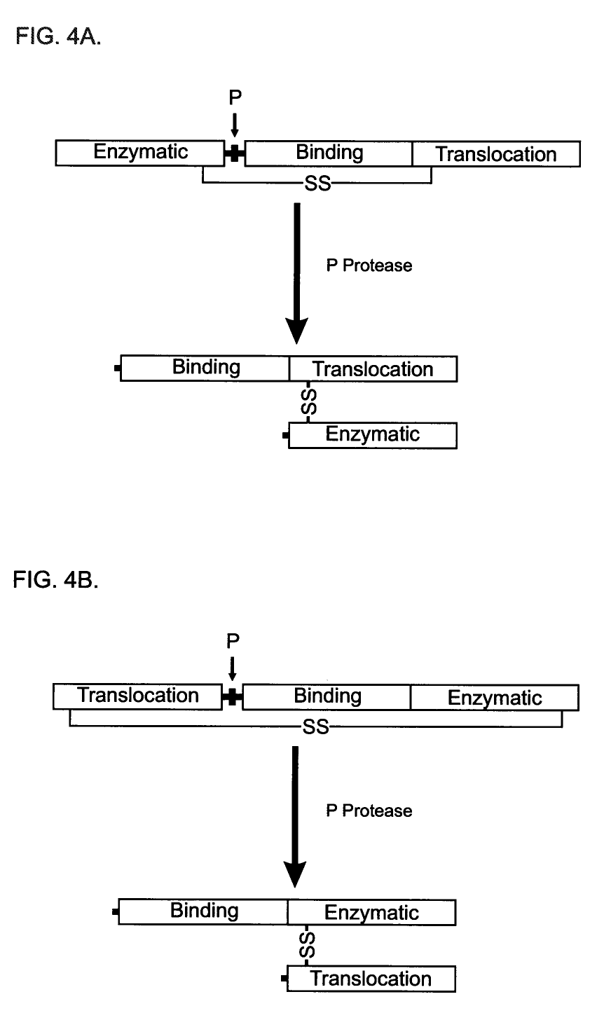

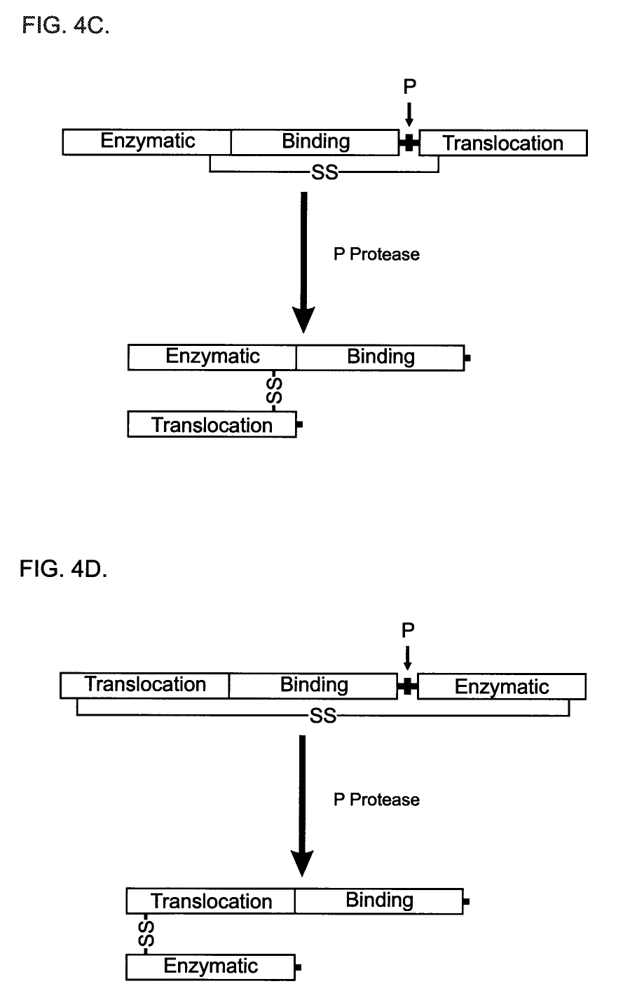

A di-chain loop region is modified to replace a naturally-occurring di-chain loop protease cleavage site for an exogenous protease cleavage site. In this modification, the naturally-occurring di-chain loop protease cleavage site is made inoperable and thus can not be cleaved by its protease. Only the exogenous protease cleavage site can be cleaved by its corresponding exogenous protease. In this type of modification, the exogenous protease site is operably-linked in-frame to a TVEMP as a fusion protein and the site can be cleaved by its respective exogenous protease. Replacement of an endogenous di-chain loop protease cleavage site with an exogenous protease cleavage site can be a substitution of the sites where the exogenous site is engineered at the position approximating the cleavage site location of the endogenous site. Replacement of an endogenous di-chain loop protease cleavage site with an exogenous protease cleavage site can be an addition of an exogenous site where the exogenous site is engineered at the position different from the cleavage site location of the endogenous site, the endogenous site being engineered to be inoperable. The location and kind of protease cleavage site may be critical because certain binding domains require a free amino-terminal or carboxyl-terminal amino acid. For example, when a retargeted peptide binding domain is placed between two other domains, e.g., see FIG. 4, a criterion for selection of a protease cleavage site could be whether the protease that cleaves its site leaves a flush cut, exposing the free amino-terminal or carboxyl-terminal of the binding domain necessary for selective binding of the binding domain to its receptor.

A naturally-occurring protease cleavage site can be made inoperable by altering at least the two amino acids flanking the peptide bond cleaved by the naturally-occurring di-chain loop protease. More extensive alterations can be made, with the proviso that the two cysteine residues of the di-chain loop region remain intact and the region can still form the disulfide bridge. Non-limiting examples of an amino acid alteration include deletion of an amino acid or replacement of the original amino acid with a different amino acid. Thus, in one embodiment, a naturally-occurring protease cleavage site is made inoperable by altering the two amino acids flanking the peptide bond cleaved by a naturally-occurring protease. In other aspects of this embodiment, a naturally-occurring protease cleavage site is made inoperable by altering, e.g., at least three amino acids including the two amino acids flanking the peptide bond cleaved by a naturally-occurring protease; at least four amino acids including the two amino acids flanking the peptide bond cleaved by a naturally-occurring protease; at least five amino acids including the two amino acids flanking the peptide bond cleaved by a naturally-occurring protease; at least six amino acids including the two amino acids flanking the peptide bond cleaved by a naturally-occurring protease; at least seven amino acids including the two amino acids flanking the peptide bond cleaved by a naturally-occurring protease; at least eight amino acids including the two amino acids flanking the peptide bond cleaved by a naturally-occurring protease; at least nine amino acids including the two amino acids flanking the peptide bond cleaved by a naturally-occurring protease; at least ten amino acids including the two amino acids flanking the peptide bond cleaved by a naturally-occurring protease; at least 15 amino acids including the two amino acids flanking the peptide bond cleaved by a naturally-occurring protease; or at least 20 amino acids including the two amino acids flanking the peptide bond cleaved by a naturally-occurring protease.

In still other aspects of this embodiment, a naturally-occurring di-chain protease cleavage site is made inoperable by altering, e.g., at most three amino acids including the two amino acids flanking the peptide bond cleaved by a naturally-occurring protease; at most four amino acids including the two amino acids flanking the peptide bond cleaved by a naturally-occurring protease; at most five amino acids including the two amino acids flanking the peptide bond cleaved by a naturally-occurring protease; at most six amino acids including the two amino acids flanking the peptide bond cleaved by a naturally-occurring protease; at most seven amino acids including the two amino acids flanking the peptide bond cleaved by a naturally-occurring protease; at most eight amino acids including the two amino acids flanking the peptide bond cleaved by a naturally-occurring protease; at most nine amino acids including the two amino acids flanking the peptide bond cleaved by a naturally-occurring protease; at most ten amino acids including the two amino acids flanking the peptide bond cleaved by a naturally-occurring protease; at most 15 amino acids including the two amino acids flanking the peptide bond cleaved by a naturally-occurring protease; or at most 20 amino acids including the two amino acids flanking the peptide bond cleaved by a naturally-occurring protease.

It is understood that a TVEMP disclosed in the present specification can optionally further comprise a flexible region comprising a flexible spacer. A flexible region comprising flexible spacers can be used to adjust the length of a polypeptide region in order to optimize a characteristic, attribute or property of a polypeptide. As a non-limiting example, a polypeptide region comprising one or more flexible spacers in tandem can be use to better expose a protease cleavage site thereby facilitating cleavage of that site by a protease. As another non-limiting example, a polypeptide region comprising one or more flexible spacers in tandem can be use to better present a retargeted peptide binding domain, thereby facilitating the binding of that binding domain to its receptor.

A flexible space comprising a peptide is at least one amino acid in length and comprises non-charged amino acids with small side-chain R groups, such as, e.g., glycine, alanine, valine, leucine or serine. Thus, in an embodiment a flexible spacer can have a length of, e.g., at least 1, 2, 3, 4, 5, 6, 7, 8, 9, or 10 amino acids; or at most 1, 2, 3, 4, 5, 6, 7, 8, 9, or 10 amino acids. In still another embodiment, a flexible spacer can be, e.g., between 1-3 amino acids, between 2-4 amino acids, between 3-5 amino acids, between 4-6 amino acids, or between 5-7 amino acids. Non-limiting examples of a flexible spacer include, e.g., a G-spacers such as GGG, GGGG (SEQ ID NO: 63), and GGGGS (SEQ ID NO: 64) or an A-spacers such as AAA, AAAA (SEQ ID NO: 65) and AAAAV (SEQ ID NO: 66). Such a flexible region is operably-linked in-frame to the TVEMP as a fusion protein.

Thus, in an embodiment, a TVEMP disclosed in the present specification can further comprise a flexible region comprising a flexible spacer. In another embodiment, a TVEMP disclosed in the present specification can further comprise flexible region comprising a plurality of flexible spacers in tandem. In aspects of this embodiment, a flexible region can comprise in tandem, e.g., at least 1, 2, 3, 4, or 5 G-spacers; or at most 1, 2, 3, 4, or 5 G-spacers. In still other aspects of this embodiment, a flexible region can comprise in tandem, e.g., at least 1, 2, 3, 4, or 5 A-spacers; or at most 1, 2, 3, 4, or 5 A-spacers. In another aspect of this embodiment, a TVEMP can comprise a flexible region comprising one or more copies of the same flexible spacers, one or more copies of different flexible-spacer regions, or any combination thereof.

In other aspects of this embodiment, a TVEMP comprising a flexible spacer can be, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

It is envisioned that a TVEMP disclosed in the present specification can comprise a flexible spacer in any and all locations with the proviso that TVEMP is capable of performing the intoxication process. In aspects of this embodiment, a flexible spacer is positioned between, e.g., an enzymatic domain and a translocation domain, an enzymatic domain and a retargeted peptide binding domain, an enzymatic domain and an exogenous protease cleavage site. In other aspects of this embodiment, a G-spacer is positioned between, e.g., an enzymatic domain and a translocation domain, an enzymatic domain and a retargeted peptide binding domain, an enzymatic domain and an exogenous protease cleavage site. In other aspects of this embodiment, an A-spacer is positioned between, e.g., an enzymatic domain and a translocation domain, an enzymatic domain and a retargeted peptide binding domain, an enzymatic domain and an exogenous protease cleavage site.

In other aspects of this embodiment, a flexible spacer is positioned between, e.g., a retargeted peptide binding domain and a translocation domain, a retargeted peptide binding domain and an enzymatic domain, a retargeted peptide binding domain and an exogenous protease cleavage site. In other aspects of this embodiment, a G-spacer is positioned between, e.g., a retargeted peptide binding domain and a translocation domain, a retargeted peptide binding domain and an enzymatic domain, a retargeted peptide binding domain and an exogenous protease cleavage site. In other aspects of this embodiment, an A-spacer is positioned between, e.g., a retargeted peptide binding domain and a translocation domain, a retargeted peptide binding domain and an enzymatic domain, a retargeted peptide binding domain and an exogenous protease cleavage site.

In yet other aspects of this embodiment, a flexible spacer is positioned between, e.g., a translocation domain and an enzymatic domain, a translocation domain and a retargeted peptide binding domain, a translocation domain and an exogenous protease cleavage site. In other aspects of this embodiment, a G-spacer is positioned between, e.g., a translocation domain and an enzymatic domain, a translocation domain and a retargeted peptide binding domain, a translocation domain and an exogenous protease cleavage site. In other aspects of this embodiment, an A-spacer is positioned between, e.g., a translocation domain and an enzymatic domain, a translocation domain and a retargeted peptide binding domain, a translocation domain and an exogenous protease cleavage site.

It is envisioned that a TVEMP disclosed in the present specification can comprise a retargeted peptide binding domain in any and all locations with the proviso that TVEMP is capable of performing the intoxication process. Non-limiting examples include, locating a retargeted peptide binding domain at the amino terminus of a TVEMP; locating a retargeted peptide binding domain between a Clostridial toxin enzymatic domain and a translocation domain of a TVEMP; and locating a retargeted peptide binding domain at the carboxyl terminus of a TVEMP. Other non-limiting examples include, locating a retargeted peptide binding domain between a Clostridial toxin enzymatic domain and a Clostridial toxin translocation domain of a TVEMP. The enzymatic domain of naturally-occurring Clostridial toxins contains the native start methionine. Thus, in domain organizations where the enzymatic domain is not in the amino-terminal location an amino acid sequence comprising the start methionine should be placed in front of the amino-terminal domain. Likewise, where a retargeted peptide binding domain is in the amino-terminal position, an amino acid sequence comprising a start methionine and a protease cleavage site may be operably-linked in situations in which a retargeted peptide binding domain requires a free amino terminus, see, e.g., Shengwen Li et al., Degradable Clostridial Toxins, U.S. patent application Ser. No. 11/572,512 (Jan. 23, 2007), which is hereby incorporated by reference in its entirety. In addition, it is known in the art that when adding a polypeptide that is operably-linked to the amino terminus of another polypeptide comprising the start methionine that the original methionine residue can be deleted.

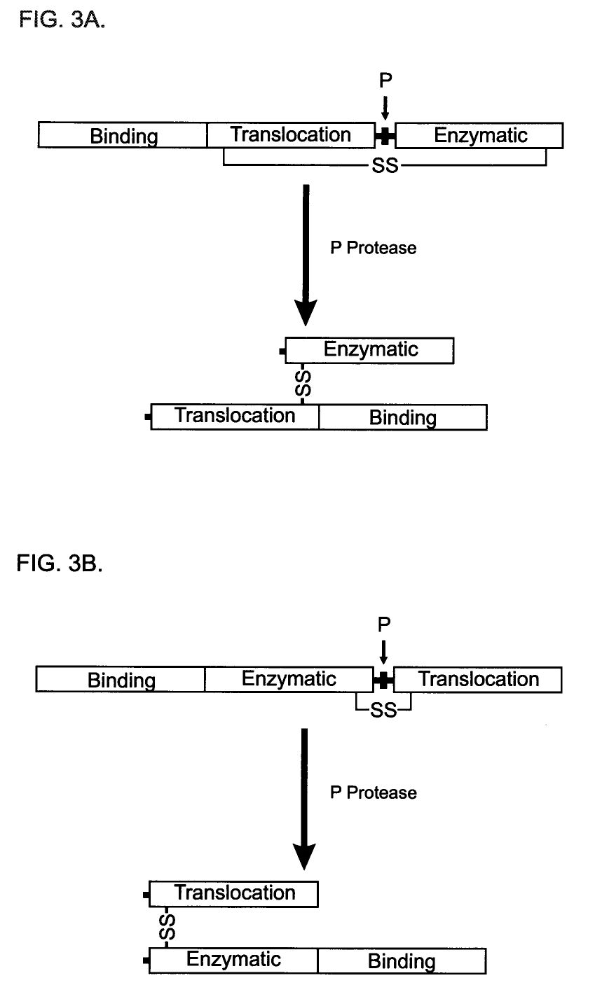

Thus, in an embodiment, a TVEMP can comprise an amino to carboxyl single polypeptide linear order comprising a retargeted peptide binding domain, a translocation domain, an exogenous protease cleavage site and an enzymatic domain (FIG. 3A). In an aspect of this embodiment, a TVEMP can comprise an amino to carboxyl single polypeptide linear order comprising a retargeted peptide binding domain, a Clostridial toxin translocation domain, an exogenous protease cleavage site and a Clostridial toxin enzymatic domain.

In another embodiment, a TVEMP can comprise an amino to carboxyl single polypeptide linear order comprising a retargeted peptide binding domain, an enzymatic domain, an exogenous protease cleavage site, and a translocation domain (FIG. 3B). In an aspect of this embodiment, a TVEMP can comprise an amino to carboxyl single polypeptide linear order comprising a retargeted peptide binding domain, a Clostridial toxin enzymatic domain, an exogenous protease cleavage site, a Clostridial toxin translocation domain.

In yet another embodiment, a TVEMP can comprise an amino to carboxyl single polypeptide linear order comprising an enzymatic domain, an exogenous protease cleavage site, a retargeted peptide binding domain, and a translocation domain (FIG. 4A). In an aspect of this embodiment, a TVEMP can comprise an amino to carboxyl single polypeptide linear order comprising a Clostridial toxin enzymatic domain, an exogenous protease cleavage site, a retargeted peptide binding domain, and a Clostridial toxin translocation domain.

In yet another embodiment, a TVEMP can comprise an amino to carboxyl single polypeptide linear order comprising a translocation domain, an exogenous protease cleavage site, a retargeted peptide binding domain, and an enzymatic domain (FIG. 4B). In an aspect of this embodiment, a TVEMP can comprise an amino to carboxyl single polypeptide linear order comprising a Clostridial toxin translocation domain, a retargeted peptide binding domain, an exogenous protease cleavage site and a Clostridial toxin enzymatic domain.

In another embodiment, a TVEMP can comprise an amino to carboxyl single polypeptide linear order comprising an enzymatic domain, a retargeted peptide binding domain, an exogenous protease cleavage site, and a translocation domain (FIG. 4C). In an aspect of this embodiment, a TVEMP can comprise an amino to carboxyl single polypeptide linear order comprising a Clostridial toxin enzymatic domain, a retargeted peptide binding domain, an exogenous protease cleavage site, a Clostridial toxin translocation domain.

In yet another embodiment, a TVEMP can comprise an amino to carboxyl single polypeptide linear order comprising a translocation domain, a retargeted peptide binding domain, an exogenous protease cleavage site and an enzymatic domain (FIG. 4D). In an aspect of this embodiment, a TVEMP can comprise an amino to carboxyl single polypeptide linear order comprising a Clostridial toxin translocation domain, a retargeted peptide binding domain, an exogenous protease cleavage site and a Clostridial toxin enzymatic domain.

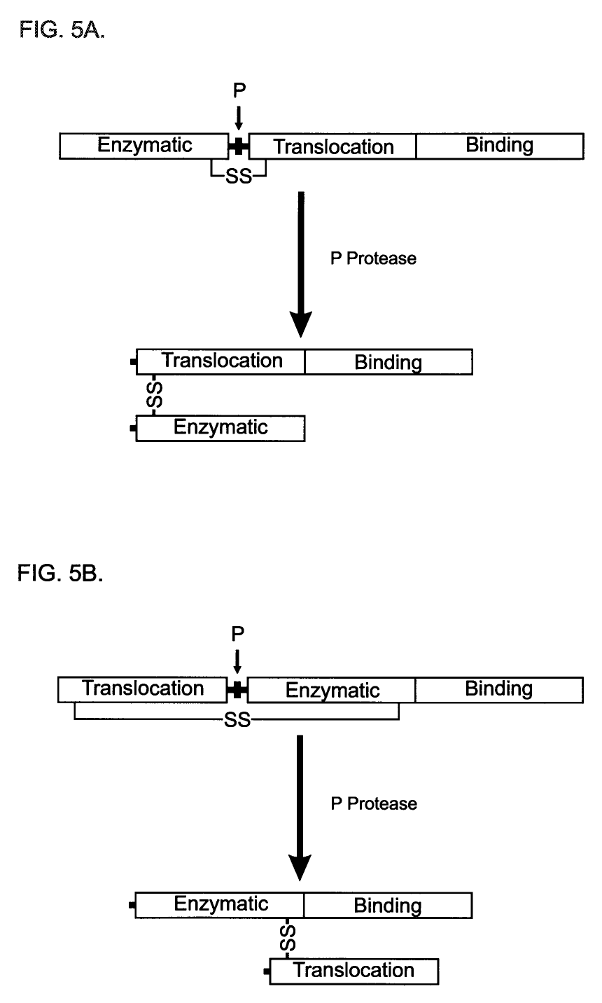

In still another embodiment, a TVEMP can comprise an amino to carboxyl single polypeptide linear order comprising an enzymatic domain, an exogenous protease cleavage site, a translocation domain, and a retargeted peptide binding domain (FIG. 5A). In an aspect of this embodiment, a TVEMP can comprise an amino to carboxyl single polypeptide linear order comprising a Clostridial toxin enzymatic domain, an exogenous protease cleavage site, a Clostridial toxin translocation domain, and a retargeted peptide binding domain.

In still another embodiment, a TVEMP can comprise an amino to carboxyl single polypeptide linear order comprising a translocation domain, an exogenous protease cleavage site, an enzymatic domain and a retargeted peptide binding domain, (FIG. 5B). In an aspect of this embodiment, a TVEMP can comprise an amino to carboxyl single polypeptide linear order comprising a Clostridial toxin translocation domain, a retargeted peptide binding domain, an exogenous protease cleavage site and a Clostridial toxin enzymatic domain.

A composition useful in the invention generally is administered as a pharmaceutical acceptable composition comprising a TVEMP. As used herein, the term “pharmaceutically acceptable” refers to any molecular entity or composition that does not produce an adverse, allergic or other untoward or unwanted reaction when administered to an individual. As used herein, the term “pharmaceutically acceptable composition” is synonymous with “pharmaceutical composition” and refers to a therapeutically effective concentration of an active ingredient, such as, e.g., any of the TVEMPs disclosed in the present specification. A pharmaceutical composition comprising a TVEMP is useful for medical and veterinary applications. A pharmaceutical composition may be administered to a patient alone, or in combination with other supplementary active ingredients, agents, drugs or hormones. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilizate, tablet, pill, pellet, capsule, powder, syrup, elixir or any other dosage form suitable for administration.

Aspects of the present invention provide, in part, a composition comprising a TVEMP. It is envisioned that any of the composition disclosed in the present specification can be useful in a method of treating urogenital-neurological disorder in a mammal in need thereof, with the proviso that the composition prevents or reduces a symptom associated with the urogenital-neurological disorder. Non-limiting examples of compositions comprising a TVEMP include a TVEMP comprising a retargeted peptide binding domain, a Clostridial toxin translocation domain and a Clostridial toxin enzymatic domain. It is envisioned that any TVEMP disclosed in the present specification can be used, including those disclosed in, e.g., Steward, supra, (2007); Dolly, supra, (2007); Foster, supra, WO 2006/059093 (2006); Foster, supra, WO 2006/059105 (Jun. 8, 2006). It is also understood that the two or more different TVEMPs can be provided as separate compositions or as part of a single composition.

It is also envisioned that a pharmaceutical composition comprising a TVEMP can optionally include a pharmaceutically acceptable carriers that facilitate processing of an active ingredient into pharmaceutically acceptable compositions. As used herein, the term “pharmacologically acceptable carrier” is synonymous with “pharmacological carrier” and refers to any carrier that has substantially no long term or permanent detrimental effect when administered and encompasses terms such as “pharmacologically acceptable vehicle, stabilizer, diluent, additive, auxiliary or excipient.” Such a carrier generally is mixed with an active compound, or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, e.g., water, saline, glycine, hyaluronic acid and the like; solid carriers such as, e.g., mannitol, lactose, starch, magnesium stearate, sodium saccharin, talcum, cellulose, glucose, sucrose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in PHARMACEUTICAL DOSAGE FORMS AND DRUG DELIVERY SYSTEMS (Howard C. Ansel et al., eds., Lippincott Williams & Wilkins Publishers, 7^{th} ed. 1999); REMINGTON: THE SCIENCE AND PRACTICE OF PHARMACY (Alfonso R. Gennaro ed., Lippincott, Williams & Wilkins, 20^{th} ed. 2000); GOODMAN & GILMAN'S THE PHARMACOLOGICAL BASIS OF THERAPEUTICS (Joel G. Hardman et al., eds., McGraw-Hill Professional, 10^{th} ed. 2001); and HANDBOOK OF PHARMACEUTICAL EXCIPIENTS (Raymond C. Rowe et al., APhA Publications, 4^{th} edition 2003). These protocols are routine procedures and any modifications are well within the scope of one skilled in the art and from the teaching herein.

It is further envisioned that a pharmaceutical composition disclosed in the present specification can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening or flavoring agents, and the like. Various buffers and methods for adjusting pH can be used to prepare a pharmaceutical composition disclosed in the present specification, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate, a stabilized oxy chloro composition, such as, e.g., PURITE® and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition useful in the invention.

In an embodiment, a composition comprising a TVEMP is a pharmaceutical composition comprising a TVEMP. In aspects of this embodiment, a pharmaceutical composition comprising a TVEMP further comprises a pharmacological carrier, a pharmaceutical component, or both a pharmacological carrier and a pharmaceutical component. In other aspects of this embodiment, a pharmaceutical composition comprising a TVEMP further comprises at least one pharmacological carrier, at least one pharmaceutical component, or at least one pharmacological carrier and at least one pharmaceutical component.

Inflammation refers to the actual tissue response (edema, erythema, etc) to a noxious stimulus. Neurogenic Inflammation refers to the fact that this tissue response is initiated and/or maintained through the release of inflammatory mediators from peripheral sensory nerve terminals (i.e., an efferent function, in contrast to the normal afferent signaling to the spinal cord in these nerves).

Aspects of the present invention provide, in part, a chronic neurogenic inflammation. As used herein, the term “chronic neurogenic inflammation” refers to an inflammatory response having pathophysiology effects where at least one of the underlying symptoms being treated is due to a nociceptive sensory nerve-based etiology, such as, e.g., the release of an inflammation inducing molecule. Chronic neurogenic inflammation includes both primary neurogenic inflammation and secondary neurogenic inflammation. As used herein, the term “primary” neurogenic inflammation refers to tissue inflammation (inflammatory symptoms) that is initiated by, or results from, the release of substances from primary sensory nerve terminals (such as C and A-delta fibers). As used herein, the term “secondary” neurogenic inflammation” refers to tissue inflammation initiated by non-neuronal sources (e.g., extravasation from vascular bed or tissue interstitium-derived, such as from mast cells or immune cells) of inflammatory mediators, such as peptides or cytokines, stimulating sensory nerve terminals and causing a release of inflammatory mediators from the nerves. These nerve-derived inflammatory mediators can, in turn, stimulate the sensory nerves as well as acting on non-neuronal targets (e.g., mast cells). The net effect of both forms (primary and secondary) of neurogenic inflammation is to have an inflammatory state that is maintained by the sensitization of the peripheral sensory nerve fibers. The physiological consequence of the resulting neurogenic inflammation depends on the tissue in question, producing, such as, e.g., cutaneous pain (allodynia, hyperalgesia), joint arthritis, visceral pain and dysfunction, pulmonary dysfunction (asthma, COPD), and bladder dysfunction (pain, overactive bladder).

As used herein, the term “inflammation inducing molecule” refers to any molecule that is released by a sensory neuron that acts in some fashion to stimulate an inflammatory response. Non-limiting examples of an inflammation inducing molecules include, without limitation, neuropeptides like substance P (SP) and calcitonin gene-related peptide (CGRP), prostaglandins, and amino acids like glutamate. As used herein, the term “inflammation mediating molecule” refers to any molecule that influences neurogenic inflammation by directly stimulating sensory nerve endings to release an inflammation inducing molecule. A molecule has a direct stimulatory effect on sensory neurons if receptors for the inflammation mediating molecule are expressed in sensory neurons. Non-limiting examples of an inflammation mediating molecules include, without limitation, histamine, bradykinin, ATP, acetylcholine, serotonin, nitric oxide, leukotrienes, cytokines, chemokines, eicosanoids, and enzymes like neutral proteases, tryptase, and lysosymes As used herein, the term “inflammation sensitizing molecule” refers to any molecule that influences neurogenic inflammation by sensitizes sensory nerve endings thereby increasing the release of an inflammation inducing molecule by a given stimulus. Non-limiting examples of an inflammation sensitizing molecules include, without limitation, prostaglandins, ATP, bradykinin, interleukin-1β, interleukin-6, tumor necrosis factor-α, nerve growth factor, serotonin, and nitric oxide.

Chronic neurogenic inflammation symptoms include, without limitation, edema, hyperemia, erythema, bruising, tenderness, stiffness, swollenness, fever, chills, stuffy nose, stuffy head, breathing problems, fluid retention, blood clots, loss of appetite, increased heart rate, formation of granulomas, fibrinous, pus, non-viscous serous fluid, or ulcer and pain. The actual symptoms associated with a chronic neurogenic inflammation are well known and can be determined by a person of ordinary skill in the art by taking into account factors, including, without limitation, the location of the neurogenic inflammation, the cause of the neurogenic inflammation, the severity of the neurogenic inflammation, the tissue or organ affected, and the associated disorder.

A chronic neurogenic inflammation symptom can be associated with a large, unrelated group of disorders which underly a variety of human diseases. Non-limiting examples of disorders exhibiting chronic neurogenic inflammation as a symptom include, without limitation, acne, acid reflux/heartburn, Alzheimer's disease, appendicitis, arteritis, arthritis, asthma, atherosclerosis, autoimmune disorders, balanitis, blepharitis, bronchiolitis, bronchitis, bursitis, cancer, carditis, celiac disease, cellulitis, cervicitis, cholangitis, cholecystitis, chorioamnionitis, chronic obstructive pulmonary disease (COPD), cirrhosis, colitis, conjunctivitis, cystitis, common cold, dacryoadenitis, dementia, dermatitis, dermatomyositis, emphysema, encephalitis, endocarditis, endometritis, enteritis, enterocolitis, epicondylitis, epididymitis, fasciitis, fibrositis, gastritis, gastroenteritis, gingivitis, glomerulonephritis, glossitis, heart disease, hepatitis, hidradenitis suppurativa, high blood pressure, ileitis, an inflammatory neuropathy, insulin resistance, interstitial cystitis, iritis, ischemic heart disease, keratitis, keratoconjunctivitis, laryngitis, mastitis, mastoiditis, meningitis, metabolic syndrome (syndrome X), a migraine, myelitis, myocarditis, myositis, nephritis, obesity, omphalitis, oophoritis, orchitis, osteochondritis, osteopenia, osteoporosis, osteitis, otitis, pancreatitis, Parkinson's disease, parotitis, a pelvic inflammatory disease, pericarditis, peritonitis, pharyngitis, phlebitis, pleuritis, pneumonitis, proctitis, prostatitis, pulpitis, pyelonephritis, pylephlebitis, rheumatic fever, rhinitis, salpingitis, sialadenitis, sinusitis, spastic colon, stomatitis, synovitis, tendonitis, tendinosis, tenosynovitis, thrombophlebitis, tonsillitis, trigonitis, a tumor, urethritis, uveitis, vaginitis, vasculitis, and vulvitis. See also, Eric R. First, Application of Botulinum Toxin to the Management of Neurogenic Inflammatory Disorders, U.S. Pat. No. 6,063,768, which is hereby incorporated by reference in its entirety.

One type of disorder exhibiting a symptom of chronic neurogenic inflammation is an arthritis. Arthritis includes a group of conditions involving damage to the joints of the body due to the inflammation of the synovium including, without limitation osteoarthritis, rheumatoid arthritis, juvenile idiopathic arthritis, spondyloarthropathies like ankylosing spondylitis, reactive arthritis (Reiter's syndrome), psoriatic arthritis, enteropathic arthritis associated with inflammatory bowel disease, Whipple disease and Behcet disease, septic arthritis, gout (also known as gouty arthritis, crystal synovitis, metabolic arthritis), pseudogout (calcium pyrophosphate deposition disease), and Still's disease. Arthritis can affect a single joint (monoarthritis), two to four joints (oligoarthritis) or five or more joints (polyarthritis) and can be either an auto-immune disease or a non-autoimmune disease.

Another type of disorder exhibiting a symptom of chronic neurogenic inflammation are autoimmune disorders. Autoimmune diseases can be broadly divided into systemic and organ-specific autoimmune disorders, depending on the principal clinico-pathologic features of each disease. Systemic autoimmune diseases include, without limitation, systemic lupus erythematosus (SLE), Sjögren's syndrome, Scleroderma, rheumatoid arthritis and polymyositis. Local autoimmune diseases may be endocrinologic (Diabetes Mellitus Type 1, Hashimoto's thyroiditis, Addison's disease etc.), dermatologic (pemphigus vulgaris), hematologic (autoimmune haemolytic anemia), neural (multiple sclerosis) or can involve virtually any circumscribed mass of body tissue. Types of autoimmune disorders include, without limitation, acute disseminated encephalomyelitis (ADEM), Addison's disease, an allergy or sensitivity, anti-phospholipid antibody syndrome (APS), arthritis, autoimmune hemolytic anemia, autoimmune hepatitis, autoimmune inner ear disease, bullous pemphigoid, celiac disease, Chagas disease, chronic obstructive pulmonary disease (COPD), diabetes mellitus type 1 (IDDM), endometriosis, fibromyalgia, Goodpasture's syndrome, Graves' disease, Guillain-Barré syndrome (GBS), Hashimoto's thyroiditis, hidradenitis suppurativa, idiopathic thrombocytopenic purpura, inflammatory bowel disease, interstitial cystitis, lupus (including discoid lupus erythematosus, drug-induced lupus erythematosus. lupus nephritis, neonatal lupus, subacute cutaneous lupus erythematosus and systemic lupus erythematosus), morphea, multiple sclerosis (MS), myasthenia gravis, myopathies, narcolepsy, neuromyotonia, pemphigus vulgaris, pernicious anaemia, primary biliary cirrhosis, recurrent disseminated encephalomyelitis (multiphasic disseminated encephalomyelitis), rheumatic fever, schizophrenia, scleroderma, Sjögren's syndrome, tenosynovitis, vasculitis, and vitiligo. See Pamela D. Van Schaack & Kenneth L. Tong, Treatment of Autoimmune Disorder with a Neurotoxin, U.S. Patent Publication 2006/138059, which is hereby incorporated by reference in its entirety.

Another type of disorder exhibiting a symptom of chronic neurogenic inflammation is an inflammatory myopathy. Inflammatory myopathies are caused by problems with the immune system attacking components of the muscle, leading to signs of inflammation in the muscle Inflammatory myopathies include, without limitation, dermatomyositis, inclusion body myositis, and polymyositis.

Another type of disorder exhibiting a symptom of chronic neurogenic inflammation is a vasculitis. Vasculitis is a varied group of disorders featuring inflammation of a vessel wall including lymphatic vessels and blood vessels like veins (phlebitis), arteries (arteritis) and capillaries due to leukocyte migration and resultant damage. The inflammation may affect any size blood vessel, anywhere in the body. It may affect either arteries and/or veins. The inflammation may be focal, meaning that it affects a single location within a vessel; or it may be widespread, with areas of inflammation scattered throughout a particular organ or tissue, or even affecting more than one organ system in the body. Vasculitis include, without limitation, Buerger's disease (thromboangiitis obliterans), cerebral vasculitis (central nervous system vasculitis), Churg-Strauss arteritis, cryoglobulinemia, essential cryoglobulinemic vasculitis, giant cell (temporal) arteritis, Golfer's vasculitis, Henoch-Schonlein purpura, hypersensitivity vasculitis (allergic vasculitis), Kawasaki disease, microscopic polyarteritis/polyangiitis, polyarteritis nodosa, polymyalgia rheumatica (PMR), rheumatoid vasculitis, Takayasu arteritis, Wegener's granulomatosis, and vasculitis secondary to connective tissue disorders like systemic lupus erythematosus (SLE), rheumatoid arthritis (RA), relapsing polychondritis, Behçet's disease, or other connective tissue disorders, vasculitis secondary to viral infection.

Another type of disorder exhibiting a symptom of chronic neurogenic inflammation is a skin disorder. Skin disorders include, without limitation, a dermatitis, including chronic actinic dermatitis, an eczema like atopic eczema, contact eczema, xerotic eczema, seborrhoeic dermatitis, dyshidrosis, discoid eczema, venous eczema, dermatitis herpetiformis, neurodermatitis, and autoeczematization, and statis dermatitis, hidradenitis suppurativa, psoriasis including plaqure psoriasis, nail psoriasis, guttate psoriasis, scalp psoriasis, inverse psoriasis, pustular psoriasis, and erythrodermis psoriasis, rosacea and scleroderma including morphea.

Another type of disorder exhibiting a symptom of chronic neurogenic inflammation is a gastrointestinal disorder. A gastrointestinal disorder includes, without limitation, irritable bowel disease, an inflammatory bowel disease including Crohn's disease and an ulcerative colitis like ulcerative proctitis, left-sided colitis, pancolitis and fulminant colitis.

Thus, in an embodiment, a mammal suffering from chronic neurogenic inflammation is treated with a composition comprising a therapeutically effective amount of a TVEMP where such administration reduces the release of an inflammation inducing molecule, thereby reducing a symptom associated with chronic neurogenic inflammation. In an aspect of this embodiment, a mammal suffering from chronic neurogenic inflammation is treated with a composition comprising a therapeutically effective amount of a TVEMP where such administration reduces the release of inflammation inducing molecule, thereby reducing a symptom associated with chronic neurogenic inflammation. In an aspect of this embodiment, a mammal suffering from a chronic neurogenic inflammation disorder is treated with a composition comprising a therapeutically effective amount of a TVEMP where such administration reduces the release of SP, thereby reducing a symptom associated with chronic neurogenic inflammation. In an aspect of this embodiment, a mammal suffering from a chronic neurogenic inflammation disorder is treated with a composition comprising a therapeutically effective amount of a TVEMP where such administration reduces the release of CGRP, thereby reducing a symptom associated with chronic neurogenic inflammation. In another aspect of this embodiment, a mammal suffering from a chronic neurogenic inflammation disorder is treated with a composition comprising a therapeutically effective amount of a TVEMP where such administration reduces the release of a prostaglandin, thereby reducing a symptom associated with chronic neurogenic inflammation. In another aspect of this embodiment, a mammal suffering from a chronic neurogenic inflammation disorder is treated with a composition comprising a therapeutically effective amount of a TVEMP where such administration reduces the release of glutamate, thereby reducing a symptom associated with chronic neurogenic inflammation.

Aspects of the present invention provide, in part, a mammal. A mammal includes a human, and a human can be a patient. Other aspects of the present invention provide, in part, an individual. An individual includes a human, and a human can be a patient.

Aspects of the present invention provide, in part, administering a composition comprising a TVEMP. As used herein, the term “administering” refers to any delivery mechanism that provides a composition comprising a TVEMP to a patient that potentially results in a clinically, therapeutically, or experimentally beneficial result. A TVEMP can be delivered to a patient using a cellular uptake approach where a TVEMP is delivered intracellular or a gene therapy approach where a TVEMP is express derived from precursor RNAs expressed from an expression vectors.

A composition comprising a TVEMP as disclosed in the present specification can be administered to a mammal using a cellular uptake approach. Administration of a composition comprising a TVEMP using a cellular uptake approach comprise a variety of enteral or parenteral approaches including, without limitation, oral administration in any acceptable form, such as, e.g., tablet, liquid, capsule, powder, or the like; topical administration in any acceptable form, such as, e.g., drops, spray, creams, gels or ointments; intravascular administration in any acceptable form, such as, e.g., intravenous bolus injection, intravenous infusion, intra-arterial bolus injection, intra-arterial infusion and catheter instillation into the vasculature; peri- and intra-tissue administration in any acceptable form, such as, e.g., intraperitoneal injection, intramuscular injection, subcutaneous injection, subcutaneous infusion, intraocular injection, retinal injection, or sub-retinal injection or epidural injection; intravesicular administration in any acceptable form, such as, e.g., catheter instillation; and by placement device, such as, e.g., an implant, a patch, a pellet, a catheter, an osmotic pump, a suppository, a bioerodible delivery system, a non-bioerodible delivery system or another implanted extended or slow release system. An exemplary list of biodegradable polymers and methods of use are described in, e.g., Handbook of Biodegradable Polymers (Abraham J. Domb et al., eds., Overseas Publishers Association, 1997).

A composition comprising a TVEMP can be administered to a mammal by a variety of methods known to those of skill in the art, including, but not restricted to, encapsulation in liposomes, by ionophoresis, or by incorporation into other vehicles, such as hydrogels, cyclodextrins, biodegradable nanocapsules, and bioadhesive microspheres, or by proteinaceous vectors. Delivery mechanisms for administering a composition comprising a TVEMP to a patient are described in, e.g., Leonid Beigelman et al., Compositions for the Delivery of Negatively Charged Molecules, U.S. Pat. No. 6,395,713 (May 28, 2002); and Achim Aigner, Delivery Systems for the Direct Application of siRNAs to Induce RNA Interference (RNAi) in vivo, 2006(716559) J. Biomed. Biotech. 1-15 (2006); Controlled Drug Delivery: Designing Technologies for the Future (Kinam Park & Randy J. Mrsny eds., American Chemical Association, 2000); Vernon G. Wong & Mae W. L. Hu, Methods for Treating Inflammation-mediated Conditions of the Eye, U.S. Pat. No. 6,726,918 (Apr. 27, 2004); David A. Weber et al., Methods and Apparatus for Delivery of Ocular Implants, U.S. Patent Publication No. US2004/0054374 (Mar. 18, 2004); Thierry Nivaggioli et al., Biodegradable Ocular Implant, U.S. Patent Publication No. US2004/0137059 (Jul. 15, 2004); Patrick M. Hughes et al., Anti-Angiogenic Sustained Release Intraocular Implants and Related Methods, U.S. patent application Ser. No. 11/364,687 (Feb. 27, 2006); and Patrick M. Hughes et al., Sustained Release Intraocular Drug Delivery Systems, U.S. Patent Publication 2006/0182783 (Aug. 17, 2006), each of which is hereby incorporated by reference in its entirety.

A composition comprising a TVEMP as disclosed in the present specification can also be administered to a patient using a gene therapy approach by expressing a TVEMP within in a cell manifesting a nerve-based etiology that contributes to a neurogenic inflammation disorder. A TVEMP can be expressed from nucleic acid molecules operably-linked to an expression vector, see, e.g., P. D. Good et al., Expression of Small, Therapeutic RNAs in Human Cell Nuclei, 4(1) Gene Ther. 45-54 (1997); James D. Thompson, Polymerase III-based expression of therapeutic RNAs, U.S. Pat. No. 6,852,535 (Feb. 8, 2005); Maciej Wiznerowicz et al., Tuning Silence: Conditional Systems for RNA Interference, 3(9) Nat. Methods 682-688m (2006); Ola Snøve and John J. Rossi, Expressing Short Hairpin RNAi in vivo, 3(9) Nat. Methods 689-698 (2006); and Charles X. Li et al., Delivery of RNA Interference, 5(18) Cell Cycle 2103-2109 (2006). A person of ordinary skill in the art would realize that any TVEMP can be expressed in eukaryotic cells using an appropriate expression vector.

Expression vectors capable of expressing a TVEMP can provide persistent or stable expression of the TVEMP in a cell manifesting a nerve-based etiology that contributes to a neurogenic inflammation disorder. Alternatively, expression vectors capable of expressing a TVEMP can provide for transient expression of the TVEMP in a cell manifesting a nerve-based etiology that contributes to a neurogenic inflammation disorder. Such transiently expressing vectors can be repeatedly administered as necessary. A TVEMP-expressing vectors can be administered by a delivery mechanism and route of administration discussed above, by administration to target cells ex-planted from a patient followed by reintroduction into the patient, or by any other method that would allow for introduction into the desired target cell, see, e.g., Larry A. Couture and Dan T. Stinchcomb, Anti-gene Therapy: The Use of Ribozymes to Inhibit Gene Function, 12(12) Trends Genet. 510-515 (1996).

The actual delivery mechanism used to administer a composition comprising a TVEMP to a mammal can be determined by a person of ordinary skill in the art by taking into account factors, including, without limitation, the type of neurogenic inflammation disorder, the location of the neurogenic inflammation disorder, the cause of the neurogenic inflammation disorder, the severity of the neurogenic inflammation disorder, the degree of relief desired, the duration of relief desired, the particular TVEMP used, the rate of excretion of the TVEMP used, the pharmacodynamics of the TVEMP used, the nature of the other compounds to be included in the composition, the particular route of administration, the particular characteristics, history and risk factors of the patient, such as, e.g., age, weight, general health and the like, or any combination thereof.

In an embodiment, a composition comprising a TVEMP is administered to the site to be treated by injection. In aspects of this embodiment, injection of a composition comprising a TVEMP is by, e.g., intramuscular injection, subdermal injection, or dermal injection. In aspects of this embodiment, injection of a composition comprising a TVEMP is into the lower urinary tract, including the bladder wall, the urinary sphincter or bladder neck.

A composition comprising a TVEMP can be administered to a mammal using a variety of routes. Routes of administration suitable for a method of treating a neurogenic inflammation disorder as disclosed in the present specification include both local and systemic administration. Local administration results in significantly more delivery of a composition to a specific location as compared to the entire body of the mammal, whereas, systemic administration results in delivery of a composition to essentially the entire body of the patient. Routes of administration suitable for a method of treating a neurogenic inflammation disorder as disclosed in the present specification also include both central and peripheral administration. Central administration results in delivery of a composition to essentially the central nervous system of the patient and includes, e.g., intrathecal administration, epidural administration as well as a cranial injection or implant. Peripheral administration results in delivery of a composition to essentially any area of a patient outside of the central nervous system and encompasses any route of administration other than direct administration to the spine or brain. The actual route of administration of a composition comprising a TVEMP used in a mammal can be determined by a person of ordinary skill in the art by taking into account factors, including, without limitation, the type of neurogenic inflammation disorder, the location of the neurogenic inflammation disorder, the cause of the neurogenic inflammation disorder, the severity of the neurogenic inflammation disorder, the degree of relief desired, the duration of relief desired, the particular TVEMP used, the rate of excretion of the TVEMP used, the pharmacodynamics of the TVEMP used, the nature of the other compounds to be included in the composition, the particular route of administration, the particular characteristics, history and risk factors of the mammal, such as, e.g., age, weight, general health and the like, or any combination thereof.

In an embodiment, a composition comprising a TVEMP is administered systemically to a mammal. In another embodiment, a composition comprising a TVEMP is administered locally to a mammal. In an aspect of this embodiment, a composition comprising a TVEMP is administered to the bladder of a mammal. In another aspect of this embodiment, a composition comprising a TVEMP is administered to the prostate of a mammal. In another aspect of this embodiment, a composition comprising a TVEMP is administered to the uterus of a mammal.

Aspects of the present invention provide, in part, administering a therapeutically effective amount of a composition comprising a TVEMP. As used herein, the term “therapeutically effective amount” is synonymous with “therapeutically effective dose” and when used in reference to treating a neurogenic inflammation disorder refers to the minimum dose of a TVEMP necessary to achieve the desired therapeutic effect and includes a dose sufficient to reduce a symptom associated with a neurogenic inflammation disorder. In aspects of this embodiment, a therapeutically effective amount of a composition comprising a TVEMP reduces a symptom associated with a neurogenic inflammation disorder by, e.g., at least 10%, at least 20%, at least 30%, at least 40%, at least 50%, at least 60%, at least 70%, at least 80%, at least 90% or at least 100%. In other aspects of this embodiment, a therapeutically effective amount of a composition comprising a TVEMP reduces a symptom associated with a neurogenic inflammation disorder by, e.g., at most 10%, at most 20%, at most 30%, at most 40%, at most 50%, at most 60%, at most 70%, at most 80%, at most 90% or at most 100%. In yet other aspects of this embodiment, a therapeutically effective amount of a composition comprising a TVEMP reduces a symptom associated with a neurogenic inflammation disorder by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In other aspects of this embodiment, a therapeutically effective amount of a composition comprising a TVEMP reduces a symptom associated with a neurogenic inflammation disorder by, e.g., about one week, about one month, about two months, about three months, about four months, about five months, about six months, about seven months, about eight months, about nine months, about ten months, about eleven months, or about twelve months. In yet other aspects of this embodiment, a therapeutically effective amount of a composition comprising a TVEMP reduces a symptom associated with a neurogenic inflammation disorder by, e.g., at least one week, at least one month, at least two months, at least three months, at least four months, at least five months, at least six months, at least seven months, at least eight months, at least nine months, at least ten months, at least eleven months, or at least twelve months. In still other aspects of this embodiment, a therapeutically effective amount of a composition comprising a TVEMP reduces a symptom associated with a neurogenic inflammation disorder by, e.g., about 1 week to about three months, about one month to about six months, about one month to about nine months, about one month to about twelve months, about three months to about six months, about three months to about nine months, about three months to about twelve months.

The actual therapeutically effective amount of a composition comprising a TVEMP to be administered to a mammal can be determined by a person of ordinary skill in the art by taking into account factors, including, without limitation, the type of neurogenic inflammation disorder, the location of the neurogenic inflammation disorder, the cause of the neurogenic inflammation disorder, the severity of the neurogenic inflammation disorder, the degree of relief desired, the duration of relief desired, the particular TVEMP used, the rate of excretion of the TVEMP used, the pharmacodynamics of the TVEMP used, the nature of the other compounds to be included in the composition, the particular route of administration, the particular characteristics, history and risk factors of the patient, such as, e.g., age, weight, general health and the like, or any combination thereof. Additionally, where repeated administration of a composition comprising a TVEMP is used, the actual effect amount of a composition comprising a TVEMP will further depend upon factors, including, without limitation, the frequency of administration, the half-life of the composition comprising a TVEMP, or any combination thereof. In is known by a person of ordinary skill in the art that an effective amount of a composition comprising a TVEMP can be extrapolated from in vitro assays and in vivo administration studies using animal models prior to administration to humans. Wide variations in the necessary effective amount are to be expected in view of the differing efficiencies of the various routes of administration. For instance, oral administration generally would be expected to require higher dosage levels than administration by intravenous or intravitreal injection. Variations in these dosage levels can be adjusted using standard empirical routines of optimization, which are well-known to a person of ordinary skill in the art. The precise therapeutically effective dosage levels and patterns are preferably determined by the attending physician in consideration of the above-identified factors.

As a non-limiting example, when administering a composition comprising a TVEMP to a mammal, a therapeutically effective amount generally is in the range of about 1 fg to about 3.0 mg. In aspects of this embodiment, an effective amount of a composition comprising a TVEMP can be, e.g., about 100 fg to about 3.0 mg, about 100 pg to about 3.0 mg, about 100 ng to about 3.0 mg, or about 100 μg to about 3.0 mg. In other aspects of this embodiment, an effective amount of a composition comprising a TVEMP can be, e.g., about 100 fg to about 750 μg, about 100 pg to about 750 μg, about 100 ng to about 750 μg, or about 1 μg to about 750 μg. In yet other aspects of this embodiment, a therapeutically effective amount of a composition comprising a TVEMP can be, e.g., at least 1 fg, at least 250 fg, at least 500 fg, at least 750 fg, at least 1 pg, at least 250 pg, at least 500 pg, at least 750 pg, at least 1 ng, at least 250 ng, at least 500 ng, at least 750 ng, at least 1 μg, at least 250 μg, at least 500 μg, at least 750 μg, or at least 1 mg. In still other aspects of this embodiment, a therapeutically effective amount of a composition comprising a TVEMP can be, e.g., at most 1 fg, at most 250 fg, at most 500 fg, at most 750 fg, at most 1 pg, at most 250 pg, at most 500 pg, at most 750 pg, at most 1 ng, at most 250 ng, at most 500 ng, at most 750 ng, at most 1 μg, at least 250 μg, at most 500 μg, at most 750 μg, or at most 1 mg.

As another non-limiting example, when administering a composition comprising a TVEMP to a mammal, a therapeutically effective amount generally is in the range of about 0.00001 mg/kg to about 3.0 mg/kg. In aspects of this embodiment, an effective amount of a composition comprising a TVEMP can be, e.g., about 0.0001 mg/kg to about 0.001 mg/kg, about 0.03 mg/kg to about 3.0 mg/kg, about 0.1 mg/kg to about 3.0 mg/kg, or about 0.3 mg/kg to about 3.0 mg/kg. In yet other aspects of this embodiment, a therapeutically effective amount of a composition comprising a TVEMP can be, e.g., at least 0.00001 mg/kg, at least 0.0001 mg/kg, at least 0.001 mg/kg, at least 0.01 mg/kg, at least 0.1 mg/kg, or at least 1 mg/kg. In yet other aspects of this embodiment, a therapeutically effective amount of a composition comprising a TVEMP can be, e.g., at most 0.00001 mg/kg, at most 0.0001 mg/kg, at most 0.001 mg/kg, at most 0.01 mg/kg, at most 0.1 mg/kg, or at most 1 mg/kg.

Dosing can be single dosage or cumulative (serial dosing), and can be readily determined by one skilled in the art. For instance, treatment of a neurogenic inflammation disorder may comprise a one-time administration of an effective dose of a composition comprising a TVEMP. As a non-limiting example, an effective dose of a composition comprising a TVEMP can be administered once to a patient, e.g., as a single injection or deposition at or near the site exhibiting a symptom of a neurogenic inflammation disorder. Alternatively, treatment of a neurogenic inflammation disorder may comprise multiple administrations of an effective dose of a composition comprising a TVEMP carried out over a range of time periods, such as, e.g., daily, once every few days, weekly, monthly or yearly. As a non-limiting example, a composition comprising a TVEMP can be administered once or twice yearly to a mammal. The timing of administration can vary from mammal to mammal, depending upon such factors as the severity of a mammal's symptoms. For example, an effective dose of a composition comprising a TVEMP can be administered to a mammal once a month for an indefinite period of time, or until the patient no longer requires therapy. A person of ordinary skill in the art will recognize that the condition of the mammal can be monitored throughout the course of treatment and that the effective amount of a composition comprising a TVEMP that is administered can be adjusted accordingly.

A composition comprising a TVEMP as disclosed in the present specification can also be administered to a mammal in combination with other therapeutic compounds to increase the overall therapeutic effect of the treatment. The use of multiple compounds to treat an indication can increase the beneficial effects while reducing the presence of side effects.

Aspects of the present invention can also be described as follows:
1. A method of treating neurogenic inflammation in a mammal, the method comprising the step of administering to the mammal in need thereof a therapeutically effective amount of a composition including a TVEMP comprising a retargeted peptide binding domain, a Clostridial toxin translocation domain and a Clostridial toxin enzymatic domain, wherein administration of the composition reduces the release of an inflammation inducing molecule, thereby reducing a symptom associated with chronic neurogenic inflammation.
2. A method of treating neurogenic inflammation in a mammal, the method comprising the step of administering to the mammal in need thereof a therapeutically effective amount of a composition including a TVEMP comprising a retargeted peptide binding domain, a Clostridial toxin translocation domain and a Clostridial toxin enzymatic domain, wherein administration of the composition reduces the release of an inflammation inducing neuropeptide, thereby reducing a symptom associated with chronic neurogenic inflammation.
3. A method of treating neurogenic inflammation in a mammal, the method comprising the step of administering to the mammal in need thereof a therapeutically effective amount of a composition including a TVEMP comprising a retargeted peptide binding domain, a Clostridial toxin translocation domain and a Clostridial toxin enzymatic domain, wherein administration of the composition reduces the release of an inflammation inducing prostaglandin or glutamate, thereby reducing a symptom associated with chronic neurogenic inflammation.
4. The method of 1-3, wherein the TVEMP comprises a linear amino-to-carboxyl single polypeptide order of 1) the Clostridial toxin enzymatic domain, the Clostridial toxin translocation domain, the retargeted peptide binding domain, 2) the Clostridial toxin enzymatic domain, the retargeted peptide binding domain, the Clostridial toxin translocation domain, 3) the retargeted peptide binding domain, the Clostridial toxin translocation domain, and the Clostridial toxin enzymatic domain, 4) the retargeted peptide binding domain, the Clostridial toxin enzymatic domain, the Clostridial toxin translocation domain, 5) the Clostridial toxin translocation domain, the Clostridial toxin enzymatic domain and the retargeted peptide binding domain, or 6) the Clostridial toxin translocation domain, the retargeted peptide binding domain and the Clostridial toxin enzymatic domain.
5. The method of 1-3, wherein the retargeted peptide binding domain is an interleukin (IL) peptide binding domain, a vascular endothelial growth factor (VEGF) peptide binding domain, an insulin-like growth factor (IGF) peptide binding domain, or an epidermal growth factor (EGF) peptide binding domain.
6. The method of 5, wherein the interleukin peptide binding domain is an IL-1, an IL-2, an IL-6, an IL-8, an IL-10, or an IL-11.
7. The method of 5, wherein the interleukin peptide binding domain comprises amino acids 123-265 of SEQ ID NO: 67, amino acids 21-153 of SEQ ID NO: 68, amino acids 57-210 of SEQ ID NO: 69, amino acids 21-99 or amino acids 31-94 of SEQ ID NO: 70, amino acids 37-173 or amino acids 19-178 of SEQ ID NO: 71, or amino acids 37-199 of SEQ ID NO: 72.
8. The method of 5, wherein the VEGF peptide binding domain is a VEGF-A, a VEGF-B, a VEGF-C, a VEGF-D, or a placenta growth factor (PIGF).
9. The method of 5, wherein the VEGF peptide binding domain comprises amino acids 50-133 of SEQ ID NO: 73, amino acids 45-127 of SEQ ID NO: 74, amino acids 129-214 of SEQ ID NO: 75, amino acids 109-194 of SEQ ID NO: 76, amino acids 46-163, amino acids 49-162, amino acids 168-345, amino acids 244-306, or amino acids 248-340 of SEQ ID NO: 77, or amino acids 50-131 or amino acids 132-203 of SEQ ID NO: 78.
10. The method of 5, wherein the IGF peptide binding domain is an IGF-1 or an IGF-2.
11. The method of 5, wherein the IGF peptide binding domain comprises amino acids 52-109 or amino acids 49-118 of SEQ ID NO: 79, or amino acids 31-84 or amino acids 25-180 of SEQ ID NO: 80.
12. The method of 5, wherein the EGF peptide binding domain an EGF, a heparin-binding EGF-like growth factor (HB-EGF), a transforming growth factor-α (TGF-α), an amphiregulin (AR), an epiregulin (EPR), an epigen (EPG), a betacellulin (BTC), a neuregulin-1 (NRG1), a neuregulin-2 (NRG2), a neuregulin-3, (NRG3), or a neuregulin-4 (NRG4).
13. The method of 12, wherein the NRG-2 is a NRG-2 isoform 1, a NRG-2 isoform 2, a NRG-2 isoform 3, a NRG-2 isoform 4, a NRG-2 isoform 5, or a NRG-2 isoform 6.
14. The method of 5, wherein the EGF peptide binding domain comprises SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91.
15. The method of 5, wherein the EGF peptide binding domain comprises amino acids 101-251 or amino acids 107-251 of SEQ ID NO: 84, amino acids 63-108 of SEQ ID NO: 85, amino acids 23-154 of SEQ ID NO: 86, amino acids 235-630 of SEQ ID NO: 88, amino acids 398-718 of SEQ ID NO: 89, or amino acids 353-648 of SEQ ID NO: 90.
16. The method of 1-3, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.
17. The method of 1-3, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.
18. The method of 1-3, wherein the neurogenic inflammation is associated with an acne, an acid reflux/heartburn, an Alzheimer's disease, an appendicitis, an arteritis, an arthritis, an asthma. an atherosclerosis, an autoimmune disorder, a balanitis, a blepharitis, a bronchiolitis, a bronchitis, a bursitis, a cancer, a carditis, a celiac disease, a cellulitis, a cervicitis, a cholangitis, a cholecystitis, a chorioamnionitis, a chronic obstructive pulmonary disease (COPD), a cirrhosis, a colitis, a conjunctivitis, a cystitis, a common cold, a dacryoadenitis, a dementia, a dermatitis, a dermatomyositis, an emphysema, an encephalitis, an endocarditis, an endometritis, an enteritis, an enterocolitis, an epicondylitis, an epididymitis, a fasciitis, a fibrositis, a gastritis, a gastroenteritis, a gingivitis, a glomerulonephritis, a glossitis, a heart disease, a hepatitis, a hidradenitis suppurativa, a high blood pressure, an ileitis, an inflammatory neuropathy, an insulin resistance, an interstitial cystitis, an iritis, an ischemic heart disease, a keratitis, a keratoconjunctivitis, a laryngitis, a mastitis, a mastoiditis, a meningitis, a metabolic syndrome (syndrome X), a migraine, a myelitis, a myocarditis, a myositis, a nephritis, an obesity, an omphalitis, an oophoritis, an orchitis, an osteochondritis, an osteopenia, an osteoporosis, an osteitis, an otitis, a pancreatitis, a Parkinson's disease, a parotitis, a pelvic inflammatory disease, a pericarditis, a peritonitis, a pharyngitis, a phlebitis, a pleuritis, a pneumonitis, a proctitis, a prostatitis, a pulpitis, a pyelonephritis, a pylephlebitis, a rheumatic fever, a rhinitis, a salpingitis, a sialadenitis, a sinusitis, a spastic colon, a stomatitis, a synovitis, a tendonitis, a tendinosis, a tenosynovitis, a thrombophlebitis, a tonsillitis, a trigonitis, a tumor, an urethritis, an uveitis, a vaginitis, a vasculitis, or a vulvitis.
19. The method of 1-3, wherein the neurogenic inflammation is associated with an arthritis.
20. The method of 19, wherein the arthritis is a monoarthritis, an oligoarthritis, or a polyarthritis.
21. The method of 19, wherein the arthritis is an auto-immune disease or a non-autoimmune disease.
22. The method of 19, wherein the arthritis is an osteoarthritis, a rheumatoid arthritis, a juvenile idiopathic arthritis, a septic arthritis, a spondyloarthropathy, a gout, a pseudogout, or Still's disease
23. The method of 22, wherein the spondyloarthropathy is an ankylosing spondylitis, a reactive arthritis (Reiter's syndrome), a psoriatic arthritis, an enteropathic arthritis associated with inflammatory bowel disease, a Whipple disease or a Behcet disease.
24. The method of 1-3, wherein the neurogenic inflammation is associated with an autoimmune disorder.
25. The method of 24, wherein the autoimmune disorder is systemic autoimmune disorder or organ-specific autoimmune disorder.
26. The method of 24, wherein the autoimmune disorder is an acute disseminated encephalomyelitis (ADEM), an Addison's disease, an allergy, an anti-phospholipid antibody syndrome (APS), an autoimmune hemolytic anemia, an autoimmune hepatitis, an autoimmune inner ear disease, a bullous pemphigoid, a celiac disease, a Chagas disease, a chronic obstructive pulmonary disease (COPD), a diabetes mellitus type 1 (IDDM), an endometriosis, a Goodpasture's syndrome, a Graves' disease, a Guillain-Barré syndrome (GBS), a Hashimoto's thyroiditis, a hidradenitis suppurativa, an idiopathic thrombocytopenic purpura, an inflammatory bowel disease, an interstitial cystitis, a lupus (including a discoid lupus erythematosus, a drug-induced lupus erythematosus. a lupus nephritis, a neonatal lupus, a subacute cutaneous lupus erythematosus and a systemic lupus erythematosus), a morphea, a multiple sclerosis (MS), a myasthenia gravis, a myopathy, a narcolepsy, a neuromyotonia, a pemphigus vulgaris, a pernicious anaemia, a primary biliary cirrhosis, a recurrent disseminated encephalomyelitis, a rheumatic fever, a schizophrenia, a scleroderma, a Sjögren's syndrome, a tenosynovitis, a vasculitis, or a vitiligo.
27. The method of 1-3, wherein the neurogenic inflammation is associated with an inflammatory myopathy.
28. The method of 27, wherein the inflammatory myopathy is a dermatomyositis, an inclusion body myositis, or a polymyositis.
29. The method of 1-3, wherein the neurogenic inflammation is associated with a vasculitis.
30. The method of 29, wherein the vasculitis is a Buerger's disease, a cerebral vasculitis, a Churg-Strauss arteritis, a cryoglobulinemia, an essential cryoglobulinemic vasculitis, a giant cell arteritis, a Golfer's vasculitis, a Henoch-Schonlein purpura, a hypersensitivity vasculitis, a Kawasaki disease, a microscopic polyarteritis/polyangiitis, a polyarteritis nodosa, a polymyalgia rheumatica (PMR), a rheumatoid vasculitis, a Takayasu arteritis, or a Wegener's granulomatosis.
31. The method of 1-3, wherein the neurogenic inflammation is associated with a skin disorder.
32. The method of 31, wherein the skin disorder is a dermatitis, an eczema, a statis dermatitis, a hidradenitis suppurativa, a psoriasis, a rosacea or a scleroderma.
33. The method of 32, wherein the eczema is an atopic eczema, a contact eczema, a xerotic eczema, a seborrhoeic dermatitis, a dyshidrosis, a discoid eczema, a venous eczema, a dermatitis herpetiformis, a neurodermatitis, or an autoeczematization.
34. The method of 32, wherein the psoriasis is a plaqure psoriasis, a nail psoriasis, a guttate psoriasis, a scalp psoriasis, an inverse psoriasis, a pustular psoriasis, or an erythrodermis psoriasis.
35. The method of 1-3, wherein the neurogenic inflammation is associated with a gastrointestinal disorder.
36. The method of 35, wherein the gastrointestinal disorder is an irritable bowel disease or an inflammatory bowel.
37. The method of 35, wherein the inflammatory bowel is a Crohn's disease or an ulcerative colitis.
38. A method of treating neurogenic inflammation in a mammal, the method comprising the step of administering to the mammal in need thereof a therapeutically effective amount of a composition including a TVEMP comprising a retargeted peptide binding domain, a Clostridial toxin translocation domain and a Clostridial toxin enzymatic domain, and an exogenous protease cleavage site, wherein administration of the composition reduces the release of an inflammation inducing molecule, thereby reducing a symptom associated with chronic neurogenic inflammation.
39. A method of treating neurogenic inflammation in a mammal, the method comprising the step of administering to the mammal in need thereof a therapeutically effective amount of a composition including a TVEMP comprising a retargeted peptide binding domain, a Clostridial toxin translocation domain and a Clostridial toxin enzymatic domain, and an exogenous protease cleavage site, wherein administration of the composition reduces the release of an inflammation inducing neuropeptide, thereby reducing a symptom associated with chronic neurogenic inflammation.
40. A method of treating neurogenic inflammation in a mammal, the method comprising the step of administering to the mammal in need thereof a therapeutically effective amount of a composition including a TVEMP comprising a retargeted peptide binding domain, a Clostridial toxin translocation domain and a Clostridial toxin enzymatic domain, and an exogenous protease cleavage site, wherein administration of the composition reduces the release of an inflammation inducing prostaglandin or glutamate, thereby reducing a symptom associated with chronic neurogenic inflammation.
41. The method of 38-40, wherein the TVEMP comprises a linear amino-to-carboxyl single polypeptide order of 1) the Clostridial toxin enzymatic domain, the exogenous protease cleavage site, the Clostridial toxin translocation domain, the retargeted peptide binding domain, 2) the Clostridial toxin enzymatic domain, the exogenous protease cleavage site, the retargeted peptide binding domain, the Clostridial toxin translocation domain, 3) the retargeted peptide binding domain, the Clostridial toxin translocation domain, the exogenous protease cleavage site and the Clostridial toxin enzymatic domain, 4) the retargeted peptide binding domain, the Clostridial toxin enzymatic domain, the exogenous protease cleavage site, the Clostridial toxin translocation domain, 5) the Clostridial toxin translocation domain, the exogenous protease cleavage site, the Clostridial toxin enzymatic domain and the retargeted peptide binding domain, or 6) the Clostridial toxin translocation domain, the exogenous protease cleavage site, the retargeted peptide binding domain and the Clostridial toxin enzymatic domain.
42. The method of 38-40, wherein the retargeted peptide binding domain is an interleukin (IL) peptide binding domain, a vascular endothelial growth factor (VEGF) peptide binding domain, an insulin-like growth factor (IGF) peptide binding domain, or an epidermal growth factor (EGF) peptide binding domain.
43. The method of 42, wherein the interleukin peptide binding domain is an IL-1, an IL-2, an IL-6, an IL-8, an IL-10, or an IL-11.
44. The method of 42, wherein the interleukin peptide binding domain comprises amino acids 123-265 of SEQ ID NO: 67, amino acids 21-153 of SEQ ID NO: 68, amino acids 57-210 of SEQ ID NO: 69, amino acids 21-99 or amino acids 31-94 of SEQ ID NO: 70, amino acids 37-173 or amino acids 19-178 of SEQ ID NO: 71, or amino acids 37-199 of SEQ ID NO: 72.
45. The method of 42, wherein the VEGF peptide binding domain is a VEGF-A, a VEGF-B, a VEGF-C, a VEGF-D, or a placenta growth factor (PIGF).
46. The method of 42, wherein the VEGF peptide binding domain comprises amino acids 50-133 of SEQ ID NO: 73, amino acids 45-127 of SEQ ID NO: 74, amino acids 129-214 of SEQ ID NO: 75, amino acids 109-194 of SEQ ID NO: 76, amino acids 46-163, amino acids 49-162, amino acids 168-345, amino acids 244-306, or amino acids 248-340 of SEQ ID NO: 77, or amino acids 50-131 or amino acids 132-203 of SEQ ID NO: 78.
47. The method of 42, wherein the IGF peptide binding domain is an IGF-1 or an IGF-2.
48. The method of 42, wherein the IGF peptide binding domain comprises amino acids 52-109 or amino acids 49-118 of SEQ ID NO: 79, or amino acids 31-84 or amino acids 25-180 of SEQ ID NO: 80.
49. The method of 42, wherein the EGF peptide binding domain an EGF, a heparin-binding EGF-like growth factor (HB-EGF), a transforming growth factor-α (TGF-α), an amphiregulin (AR), an epiregulin (EPR), an epigen (EPG), a betacellulin (BTC), a neuregulin-1 (NRG1), a neuregulin-2 (NRG2), a neuregulin-3, (NRG3), or a neuregulin-4 (NRG4).
50. The method of 49, wherein the NRG-2 is a NRG-2 isoform 1, a NRG-2 isoform 2, a NRG-2 isoform 3, a NRG-2 isoform 4, a NRG-2 isoform 5, or a NRG-2 isoform 6.
51. The method of 42, wherein the EGF peptide binding domain comprises SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91.
52. The method of 42, wherein the EGF peptide binding domain comprises amino acids 101-251 or amino acids 107-251 of SEQ ID NO: 84, amino acids 63-108 of SEQ ID NO: 85, amino acids 23-154 of SEQ ID NO: 86, amino acids 235-630 of SEQ ID NO: 88, amino acids 398-718 of SEQ ID NO: 89, or amino acids 353-648 of SEQ ID NO: 90.
53. The method of 38-40, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.
54. The method of 38-40, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.
55. The method of 38-40, wherein the exogenous protease cleavage site is a plant papain cleavage site, an insect papain cleavage site, a crustacian papain cleavage site, an enterokinase cleavage site, a human rhinovirus 3C protease cleavage site, a human enterovirus 3C protease cleavage site, a tobacco etch virus protease cleavage site, a Tobacco Vein Mottling Virus cleavage site, a subtilisin cleavage site, a hydroxylamine cleavage site, or a Caspase 3 cleavage site.
56. The method of 38-40, wherein the neurogenic inflammation is associated with an acne, an acid reflux/heartburn, an Alzheimer's disease, an appendicitis, an arteritis, an arthritis, an asthma. an atherosclerosis, an autoimmune disorder, a balanitis, a blepharitis, a bronchiolitis, a bronchitis, a bursitis, a cancer, a carditis, a celiac disease, a cellulitis, a cervicitis, a cholangitis, a cholecystitis, a chorioamnionitis, a chronic obstructive pulmonary disease (COPD), a cirrhosis, a colitis, a conjunctivitis, a cystitis, a common cold, a dacryoadenitis, a dementia, a dermatitis, a dermatomyositis, an emphysema, an encephalitis, an endocarditis, an endometritis, an enteritis, an enterocolitis, an epicondylitis, an epididymitis, a fasciitis, a fibrositis, a gastritis, a gastroenteritis, a gingivitis, a glomerulonephritis, a glossitis, a heart disease, a hepatitis, a hidradenitis suppurativa, a high blood pressure, an ileitis, an inflammatory neuropathy, an insulin resistance, an interstitial cystitis, an iritis, an ischemic heart disease, a keratitis, a keratoconjunctivitis, a laryngitis, a mastitis, a mastoiditis, a meningitis, a metabolic syndrome (syndrome X), a migraine, a myelitis, a myocarditis, a myositis, a nephritis, an obesity, an omphalitis, an oophoritis, an orchitis, an osteochondritis, an osteopenia, an osteoporosis, an osteitis, an otitis, a pancreatitis, a Parkinson's disease, a parotitis, a pelvic inflammatory disease, a pericarditis, a peritonitis, a pharyngitis, a phlebitis, a pleuritis, a pneumonitis, a proctitis, a prostatitis, a pulpitis, a pyelonephritis, a pylephlebitis, a rheumatic fever, a rhinitis, a salpingitis, a sialadenitis, a sinusitis, a spastic colon, a stomatitis, a synovitis, a tendonitis, a tendinosis, a tenosynovitis, a thrombophlebitis, a tonsillitis, a trigonitis, a tumor, an urethritis, an uveitis, a vaginitis, a vasculitis, or a vulvitis.
57. The method of 56, wherein the neurogenic inflammation is associated with an arthritis.
58. The method of 56, wherein the arthritis is a monoarthritis, an oligoarthritis, or a polyarthritis.
59. The method of 56, wherein the arthritis is an auto-immune disease or a non-autoimmune disease.
60. The method of 56, wherein the arthritis is an osteoarthritis, a rheumatoid arthritis, a juvenile idiopathic arthritis, a septic arthritis, a spondyloarthropathy, a gout, a pseudogout, or Still's disease
61. The method of 60, wherein the spondyloarthropathy is an ankylosing spondylitis, a reactive arthritis (Reiter's syndrome), a psoriatic arthritis, an enteropathic arthritis associated with inflammatory bowel disease, a Whipple disease or a Behcet disease.
62. The method of 38-40, wherein the neurogenic inflammation is associated with an autoimmune disorder.
63. The method of 62, wherein the autoimmune disorder is systemic autoimmune disorder or organ-specific autoimmune disorder.
64. The method of 62, wherein the autoimmune disorder is an acute disseminated encephalomyelitis (ADEM), an Addison's disease, an allergy, an anti-phospholipid antibody syndrome (APS), an autoimmune hemolytic anemia, an autoimmune hepatitis, an autoimmune inner ear disease, a bullous pemphigoid, a celiac disease, a Chagas disease, a chronic obstructive pulmonary disease (COPD), a diabetes mellitus type 1 (IDDM), an endometriosis, a Goodpasture's syndrome, a Graves' disease, a Guillain-Barré syndrome (GBS), a Hashimoto's thyroiditis, a hidradenitis suppurativa, an idiopathic thrombocytopenic purpura, an inflammatory bowel disease, an interstitial cystitis, a lupus (including a discoid lupus erythematosus, a drug-induced lupus erythematosus. a lupus nephritis, a neonatal lupus, a subacute cutaneous lupus erythematosus and a systemic lupus erythematosus), a morphea, a multiple sclerosis (MS), a myasthenia gravis, a myopathy, a narcolepsy, a neuromyotonia, a pemphigus vulgaris, a pernicious anaemia, a primary biliary cirrhosis, a recurrent disseminated encephalomyelitis, a rheumatic fever, a schizophrenia, a scleroderma, a Sjögren's syndrome, a tenosynovitis, a vasculitis, or a vitiligo.
65. The method of 38-40, wherein the neurogenic inflammation is associated with an inflammatory myopathy.
66. The method of 65, wherein the inflammatory myopathy is a dermatomyositis, an inclusion body myositis, or a polymyositis.
67. The method of 38-40, wherein the neurogenic inflammation is associated with a vasculitis.
68. The method of 67, wherein the vasculitis is a Buerger's disease, a cerebral vasculitis, a Churg-Strauss arteritis, a cryoglobulinemia, an essential cryoglobulinemic vasculitis, a giant cell arteritis, a Golfer's vasculitis, a Henoch-Schonlein purpura, a hypersensitivity vasculitis, a Kawasaki disease, a microscopic polyarteritis/polyangiitis, a polyarteritis nodosa, a polymyalgia rheumatica (PMR), a rheumatoid vasculitis, a Takayasu arteritis, or a Wegener's granulomatosis.
69. The method of 38-40, wherein the neurogenic inflammation is associated with a skin disorder.
70. The method of 69, wherein the skin disorder is a dermatitis, an eczema, a statis dermatitis, a hidradenitis suppurativa, a psoriasis, a rosacea or a scleroderma.
71. The method of 70, wherein the eczema is an atopic eczema, a contact eczema, a xerotic eczema, a seborrhoeic dermatitis, a dyshidrosis, a discoid eczema, a venous eczema, a dermatitis herpetiformis, a neurodermatitis, or an autoeczematization.
72. The method of 70, wherein the psoriasis is a plaqure psoriasis, a nail psoriasis, a guttate psoriasis, a scalp psoriasis, an inverse psoriasis, a pustular psoriasis, or an erythrodermis psoriasis.
73. The method of 38-40, wherein the neurogenic inflammation is associated with a gastrointestinal disorder.
74. The method of 73, wherein the gastrointestinal disorder is an irritable bowel disease or an inflammatory bowel.
75. The method of 74, wherein the inflammatory bowel is a Crohn's disease or an ulcerative colitis.
76. A manufacturing a medicament for treating chronic neurogenic inflammation in a mammal in need thereof, wherein the medicament comprises a TVEMP including a retargeted peptide binding domain, a Clostridial toxin translocation domain and a Clostridial toxin enzymatic domain and wherein administration of a therapeutically effective amount of the medicament to the mammal reduces a symptom associated with chronic neurogenic inflammation, thereby treating chronic neurogenic inflammation.
77. A manufacturing a medicament for treating chronic neurogenic inflammation in a mammal in need thereof, wherein the medicament comprises a TVEMP including a retargeted peptide binding domain, a Clostridial toxin translocation domain and a Clostridial toxin enzymatic domain and wherein administration of a therapeutically effective amount of the medicament to the mammal reduces the release of an inflammation inducing molecule, thereby reducing a symptom associated with chronic neurogenic inflammation.
78. A manufacturing a medicament for treating chronic neurogenic inflammation in a mammal in need thereof, wherein the medicament comprises a TVEMP including a retargeted peptide binding domain, a Clostridial toxin translocation domain and a Clostridial toxin enzymatic domain and wherein administration of a therapeutically effective amount of the medicament to the mammal reduces the release of an inflammation inducing neuropeptide, thereby reducing a symptom associated with chronic neurogenic inflammation.
79. A manufacturing a medicament for treating chronic neurogenic inflammation in a mammal in need thereof, wherein the medicament comprises a TVEMP including a retargeted peptide binding domain, a Clostridial toxin translocation domain, a Clostridial toxin enzymatic domain, and an exogenous protease cleavage site and wherein administration of a therapeutically effective amount of the medicament to the mammal reduces a symptom associated with chronic neurogenic inflammation, thereby treating chronic neurogenic inflammation.
80. A manufacturing a medicament for treating chronic neurogenic inflammation in a mammal in need thereof, wherein the medicament comprises a TVEMP including a retargeted peptide binding domain, a Clostridial toxin translocation domain, a Clostridial toxin enzymatic domain, and an exogenous protease cleavage site and wherein administration of a therapeutically effective amount of the medicament to the mammal reduces the release of an inflammation inducing molecule, thereby reducing a symptom associated with chronic neurogenic inflammation.
81. A manufacturing a medicament for treating chronic neurogenic inflammation in a mammal in need thereof, wherein the medicament comprises a TVEMP including a retargeted peptide binding domain, a Clostridial toxin translocation domain, a Clostridial toxin enzymatic domain, and an exogenous protease cleavage site and wherein administration of a therapeutically effective amount of the medicament to the mammal reduces the release of an inflammation inducing neuropeptide, thereby reducing a symptom associated with chronic neurogenic inflammation.
82. A use of a composition for the treatment of chronic neurogenic inflammation in a mammal in need thereof, the use comprising the step of administering to the mammal a therapeutically effective amount of the composition, wherein the composition comprises a TVEMP including a retargeted peptide binding domain, a Clostridial toxin translocation domain, a Clostridial toxin enzymatic domain and wherein administration of the composition reduces a symptom associated with chronic neurogenic inflammation, thereby treating chronic neurogenic inflammation.
83. A use of a composition for the treatment of chronic neurogenic inflammation in a mammal in need thereof, the use comprising the step of administering to the mammal a therapeutically effective amount of the composition, wherein the composition comprises a TVEMP including a retargeted peptide binding domain, a Clostridial toxin translocation domain, a Clostridial toxin enzymatic domain and wherein administration of the composition reduces the release of an inflammation inducing molecule, thereby reducing a symptom associated with chronic neurogenic inflammation.
84. A use of a composition for the treatment of chronic neurogenic inflammation in a mammal in need thereof, the use comprising the step of administering to the mammal a therapeutically effective amount of the composition, wherein the composition comprises a TVEMP including a retargeted peptide binding domain, a Clostridial toxin translocation domain, a Clostridial toxin enzymatic domain and wherein administration of the composition reduces the release of an inflammation inducing neuropeptide, thereby reducing a symptom associated with chronic neurogenic inflammation.
85. A use of a composition for the treatment of chronic neurogenic inflammation in a mammal in need thereof, the use comprising the step of administering to the mammal a therapeutically effective amount of the composition, wherein the composition comprises a TVEMP including a retargeted peptide binding domain, a Clostridial toxin translocation domain, a Clostridial toxin enzymatic domain, and an exogenous protease cleavage site and wherein administration of the composition reduces a symptom associated with chronic neurogenic inflammation, thereby treating chronic neurogenic inflammation.
86. A use of a composition for the treatment of chronic neurogenic inflammation in a mammal in need thereof, the use comprising the step of administering to the mammal a therapeutically effective amount of the composition, wherein the composition comprises a TVEMP including a retargeted peptide binding domain, a Clostridial toxin translocation domain, a Clostridial toxin enzymatic domain, and an exogenous protease cleavage site and wherein administration of the composition reduces the release of an inflammation inducing molecule, thereby reducing a symptom associated with chronic neurogenic inflammation.
87. A use of a composition for the treatment of chronic neurogenic inflammation in a mammal in need thereof, the use comprising the step of administering to the mammal a therapeutically effective amount of the composition, wherein the composition comprises a TVEMP including a retargeted peptide binding domain, a Clostridial toxin translocation domain, a Clostridial toxin enzymatic domain, and an exogenous protease cleavage site and wherein administration of the composition reduces the release of an inflammation inducing neuropeptide, thereby reducing a symptom associated with chronic neurogenic inflammation.
88. The medicament of 77 or 80, or use of 83, or 86, wherein the inflammation inducing molecule is an inflammation inducing prostaglandin or glutamate.
89. The medicament of 79-81 or use of 85-87, wherein the TVEMP comprises a linear amino-to-carboxyl single polypeptide order of 1) the Clostridial toxin enzymatic domain, the exogenous protease cleavage site, the Clostridial toxin translocation domain, the retargeted peptide binding domain, 2) the Clostridial toxin enzymatic domain, the exogenous protease cleavage site, the retargeted peptide binding domain, the Clostridial toxin translocation domain, 3) the retargeted peptide binding domain, the Clostridial toxin translocation domain, the exogenous protease cleavage site and the Clostridial toxin enzymatic domain, 4) the retargeted peptide binding domain, the Clostridial toxin enzymatic domain, the exogenous protease cleavage site, the Clostridial toxin translocation domain, 5) the Clostridial toxin translocation domain, the exogenous protease cleavage site, the Clostridial toxin enzymatic domain and the retargeted peptide binding domain, or 6) the Clostridial toxin translocation domain, the exogenous protease cleavage site, the retargeted peptide binding domain and the Clostridial toxin enzymatic domain.
90. The medicament of 76-81 or use of 82-87, wherein the retargeted peptide binding domain is an interleukin (IL) peptide binding domain, a vascular endothelial growth factor (VEGF) peptide binding domain, an insulin-like growth factor (IGF) peptide binding domain, or an epidermal growth factor (EGF) peptide binding domain.
91. The medicament or the use of 90, wherein the interleukin peptide binding domain is an IL-1, an IL-2, an IL-6, an IL-8, an IL-10, or an IL-11.
92. The medicament or the use of 90, wherein the interleukin peptide binding domain comprises amino acids 123-265 of SEQ ID NO: 67, amino acids 21-153 of SEQ ID NO: 68, amino acids 57-210 of SEQ ID NO: 69, amino acids 21-99 or amino acids 31-94 of SEQ ID NO: 70, amino acids 37-173 or amino acids 19-178 of SEQ ID NO: 71, or amino acids 37-199 of SEQ ID NO: 72.
93. The medicament or the use of 90, wherein the VEGF peptide binding domain is a VEGF-A, a VEGF-B, a VEGF-C, a VEGF-D, or a placenta growth factor (PIGF).
94. The medicament or the use of 90, wherein the VEGF peptide binding domain comprises amino acids 50-133 of SEQ ID NO: 73, amino acids 45-127 of SEQ ID NO: 74, amino acids 129-214 of SEQ ID NO: 75, amino acids 109-194 of SEQ ID NO: 76, amino acids 46-163, amino acids 49-162, amino acids 168-345, amino acids 244-306, or amino acids 248-340 of SEQ ID NO: 77, or amino acids 50-131 or amino acids 132-203 of SEQ ID NO: 78.
95. The medicament or the use of 90, wherein the IGF peptide binding domain is an IGF-1 or an IGF-2.
96. The medicament or the use of 90, wherein the IGF peptide binding domain comprises amino acids 52-109 or amino acids 49-118 of SEQ ID NO: 79, or amino acids 31-84 or amino acids 25-180 of SEQ ID NO: 80.
97. The medicament or the use of 90, wherein the EGF peptide binding domain an EGF, a heparin-binding EGF-like growth factor (HB-EGF), a transforming growth factor-α (TGF-α), an amphiregulin (AR), an epiregulin (EPR), an epigen (EPG), a betacellulin (BTC), a neuregulin-1 (NRG1), a neuregulin-2 (NRG2), a neuregulin-3, (NRG3), or a neuregulin-4 (NRG4).
98. The medicament or the use of 97, wherein the NRG-2 is a NRG-2 isoform 1, a NRG-2 isoform 2, a NRG-2 isoform 3, a NRG-2 isoform 4, a NRG-2 isoform 5, or a NRG-2 isoform 6.
99. The medicament or the use of 90, wherein the EGF peptide binding domain comprises SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91.
100. The medicament or the use of 90, wherein the EGF peptide binding domain comprises amino acids 101-251 or amino acids 107-251 of SEQ ID NO: 84, amino acids 63-108 of SEQ ID NO: 85, amino acids 23-154 of SEQ ID NO: 86, amino acids 235-630 of SEQ ID NO: 88, amino acids 398-718 of SEQ ID NO: 89, or amino acids 353-648 of SEQ ID NO: 90.
101. The medicament of 76-81 or use of 82-87, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.
102. The medicament of 76-81 or use of 82-87, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.
103. The medicament of 79-81 or use of 85-87, wherein the exogenous protease cleavage site is a plant papain cleavage site, an insect papain cleavage site, a crustacian papain cleavage site, an enterokinase cleavage site, a human rhinovirus 3C protease cleavage site, a human enterovirus 3C protease cleavage site, a tobacco etch virus protease cleavage site, a Tobacco Vein Mottling Virus cleavage site, a subtilisin cleavage site, a hydroxylamine cleavage site, or a Caspase 3 cleavage site.
104. The medicament of 76-81 or use of 82-87, wherein the neurogenic inflammation is associated with an acne, an acid reflux/heartburn, an Alzheimer's disease, an appendicitis, an arteritis, an arthritis, an asthma. an atherosclerosis, an autoimmune disorder, a balanitis, a blepharitis, a bronchiolitis, a bronchitis, a bursitis, a cancer, a carditis, a celiac disease, a cellulitis, a cervicitis, a cholangitis, a cholecystitis, a chorioamnionitis, a chronic obstructive pulmonary disease (COPD), a cirrhosis, a colitis, a conjunctivitis, a cystitis, a common cold, a dacryoadenitis, a dementia, a dermatitis, a dermatomyositis, an emphysema, an encephalitis, an endocarditis, an endometritis, an enteritis, an enterocolitis, an epicondylitis, an epididymitis, a fasciitis, a fibrositis, a gastritis, a gastroenteritis, a gingivitis, a glomerulonephritis, a glossitis, a heart disease, a hepatitis, a hidradenitis suppurativa, a high blood pressure, an ileitis, an inflammatory neuropathy, an insulin resistance, an interstitial cystitis, an iritis, an ischemic heart disease, a keratitis, a keratoconjunctivitis, a laryngitis, a mastitis, a mastoiditis, a meningitis, a metabolic syndrome (syndrome X), a migraine, a myelitis, a myocarditis, a myositis, a nephritis, an obesity, an omphalitis, an oophoritis, an orchitis, an osteochondritis, an osteopenia, an osteoporosis, an osteitis, an otitis, a pancreatitis, a Parkinson's disease, a parotitis, a pelvic inflammatory disease, a pericarditis, a peritonitis, a pharyngitis, a phlebitis, a pleuritis, a pneumonitis, a proctitis, a prostatitis, a pulpitis, a pyelonephritis, a pylephlebitis, a rheumatic fever, a rhinitis, a salpingitis, a sialadenitis, a sinusitis, a spastic colon, a stomatitis, a synovitis, a tendonitis, a tendinosis, a tenosynovitis, a thrombophlebitis, a tonsillitis, a trigonitis, a tumor, an urethritis, an uveitis, a vaginitis, a vasculitis, or a vulvitis.
105. The medicament of 76-81 or use of 82-87, wherein the neurogenic inflammation is associated with an arthritis.
106. The medicament or use of 105, wherein the arthritis is a monoarthritis, an oligoarthritis, or a polyarthritis.
107. The medicament or use of 105, wherein the arthritis is an auto-immune disease or a non-autoimmune disease.
108. The medicament or use of 105, wherein the arthritis is an osteoarthritis, a rheumatoid arthritis, a juvenile idiopathic arthritis, a septic arthritis, a spondyloarthropathy, a gout, a pseudogout, or Still's disease
109. The medicament or use of 108, wherein the spondyloarthropathy is an ankylosing spondylitis, a reactive arthritis (Reiter's syndrome), a psoriatic arthritis, an enteropathic arthritis associated with inflammatory bowel disease, a Whipple disease or a Behcet disease.
110. The medicament of 76-81 or use of 82-87, wherein the neurogenic inflammation is associated with an autoimmune disorder.
111. The medicament or use of 110, wherein the autoimmune disorder is systemic autoimmune disorder or organ-specific autoimmune disorder.
112. The medicament or use of 110, wherein the autoimmune disorder is an acute disseminated encephalomyelitis (ADEM), an Addison's disease, an allergy, an anti-phospholipid antibody syndrome (APS), an autoimmune hemolytic anemia, an autoimmune hepatitis, an autoimmune inner ear disease, a bullous pemphigoid, a celiac disease, a Chagas disease, a chronic obstructive pulmonary disease (COPD), a diabetes mellitus type 1 (IDDM), an endometriosis, a Goodpasture's syndrome, a Graves' disease, a Guillain-Barré syndrome (GBS), a Hashimoto's thyroiditis, a hidradenitis suppurativa, an idiopathic thrombocytopenic purpura, an inflammatory bowel disease, an interstitial cystitis, a lupus (including a discoid lupus erythematosus, a drug-induced lupus erythematosus. a lupus nephritis, a subacute cutaneous lupus erythematosus a neonatal lupus, and a systemic lupus erythematosus), a morphea, a multiple sclerosis (MS), a myasthenia gravis, a myopathy, a narcolepsy, a neuromyotonia, a pemphigus vulgaris, a pernicious anaemia, a primary biliary cirrhosis, a recurrent disseminated encephalomyelitis, a rheumatic fever, a schizophrenia, a scleroderma, a Sjögren's syndrome, a tenosynovitis, a vasculitis, or a vitiligo.
113. The medicament of 76-81 or use of 82-87, wherein the neurogenic inflammation is associated with an inflammatory myopathy.
114. The medicament or use of 113, wherein the inflammatory myopathy is a dermatomyositis, an inclusion body myositis, or a polymyositis.
115. The medicament of 76-81 or use of 82-87, wherein the neurogenic inflammation is associated with a vasculitis.
116. The medicament or use of 115, wherein the vasculitis is a Buerger's disease, a cerebral vasculitis, a Churg-Strauss arteritis, a cryoglobulinemia, an essential cryoglobulinemic vasculitis, a giant cell arteritis, a Golfer's vasculitis, a Henoch-Schonlein purpura, a hypersensitivity vasculitis, a Kawasaki disease, a microscopic polyarteritis/polyangiitis, a polyarteritis nodosa, a polymyalgia rheumatica (PMR), a rheumatoid vasculitis, a Takayasu arteritis, or a Wegener's granulomatosis.
117. The medicament of 76-81 or use of 82-87, wherein the neurogenic inflammation is associated with a skin disorder.
118. The medicament or use of 117, wherein the skin disorder is a dermatitis, an eczema, a statis dermatitis, a hidradenitis suppurativa, a psoriasis, a rosacea or a scleroderma.
119. The medicament or use of 117, wherein the eczema is an atopic eczema, a contact eczema, a xerotic eczema, a seborrhoeic dermatitis, a dyshidrosis, a discoid eczema, a venous eczema, a dermatitis herpetiformis, a neurodermatitis, or an autoeczematization.
120. The medicament or use of 117, wherein the psoriasis is a plaqure psoriasis, a nail psoriasis, a guttate psoriasis, a scalp psoriasis, an inverse psoriasis, a pustular psoriasis, or an erythrodermis psoriasis.
121. The medicament of 76-81 or use of 82-87, wherein the neurogenic inflammation is associated with a gastrointestinal disorder.
122. The medicament or use of 121, wherein the gastrointestinal disorder is an irritable bowel disease or an inflammatory bowel.
123. The medicament or use of 121, wherein the inflammatory bowel is a Crohn's disease or an ulcerative colitis.
124. The medicament of 76-78 or use of 82-84, wherein the TVEMP comprises a linear amino-to-carboxyl single polypeptide order of 1) the Clostridial toxin enzymatic domain, the Clostridial toxin translocation domain, the retargeted peptide binding domain, 2) the Clostridial toxin enzymatic domain, the retargeted peptide binding domain, the Clostridial toxin translocation domain, 3) the retargeted peptide binding domain, the Clostridial toxin translocation domain, and the Clostridial toxin enzymatic domain, 4) the retargeted peptide binding domain, the Clostridial toxin enzymatic domain, the Clostridial toxin translocation domain, 5) the Clostridial toxin translocation domain, the Clostridial toxin enzymatic domain and the retargeted peptide binding domain, or 6) the Clostridial toxin translocation domain, the retargeted peptide binding domain and the Clostridial toxin enzymatic domain.

### EXAMPLES

The following non-limiting examples are provided for illustrative purposes only in order to facilitate a more complete understanding of disclosed embodiments and are in no way intended to limit any of the embodiments disclosed in the present specification.

### Example 1

### Treatment of Chronic Neurogenic Inflammation

A 62 year old female diagnosed with rheumatoid arthritis complains of joint stiffness and swelling. A physician determines that the joint stiffness and swelling is due to chronic neurogenic inflammation. The woman is treated by local administration a composition comprising a TVEMP as disclosed in the present specification in the vicinity of the affected area. The patient's condition is monitored and after about 1-3 days after treatment, and the woman indicates there is reduced joint stiffness and swelling. At one and three month check-ups, the woman indicates that she continues to have reduced joint stiffness and swelling in the area treated. This reduction in chronic neurogenic inflammation symptoms indicates successful treatment with the composition comprising a TVEMP. A similar type of local administration of a TVEMP as disclosed in the present specification can be used to treat a patient suffering from chronic neurogenic inflammation associated with any monoarthritis, oligoarthritis, or polyarthritis, such as, e.g., osteoarthritis, juvenile idiopathic arthritis, septic arthritis, a spondyloarthropathy (including ankylosing spondylitis, reactive arthritis (Reiter's syndrome), psoriatic arthritis, enteropathic arthritis associated with inflammatory bowel disease, Whipple disease or Behcet disease), a synovitis, gout, pseudogout, or Still's disease, as well as, a bursitis, a rheumatic fever, or a tenosynovitis. In addition, systemic administration could also be used to administer a disclosed TVEMP to treat chronic neurogenic inflammation.

A 58 year old male diagnosed with chronic obstructive pulmonary disease (COPD) complains of breathing difficulty. A physician determines that the breathing difficulty is due to chronic neurogenic inflammation. The man is treated by systemically by intravenous administration a composition comprising a TVEMP as disclosed in the present specification. The patient's condition is monitored and after about 1-3 days after treatment, and the man indicates there is improvement in his ability to breath. At one and three month check-ups, the man indicates that he continues to have improved breathing. This reduction in a chronic neurogenic inflammation symptom indicates successful treatment with the composition comprising a TVEMP. A similar type of systemic administration of a TVEMP as disclosed in the present specification can be used to treat a patient suffering from chronic neurogenic inflammation associated with an asthma, a bronchiolitis, a bronchitis, an emphysema, a laryngitis, a pharyngitis, a pleuritis, a pneumonitis, a rhinitis, a sinusitis, or any other type of chronic respiratory disorder. In addition, administration by inhalation could also be used to administer a disclosed TVEMP to treat chronic neurogenic inflammation.

A 67 year old male diagnosed with dermatomyositis complains of muscle soreness. A physician determines that the soreness is due to chronic neurogenic inflammation. The man is treated by local administration a composition comprising a TVEMP as disclosed in the present specification in the vicinity of the affected area. The patient's condition is monitored and after about 1-3 days after treatment, and the man indicates there is reduced soreness. At one and three month check-ups, the man indicates that he continues to have improved muscle movement and reduced soreness This reduction in a chronic neurogenic inflammation symptom indicates successful treatment with the composition comprising a TVEMP. A similar type of local administration of a TVEMP as disclosed in the present specification can be used to treat a patient suffering from chronic neurogenic inflammation associated with an inclusion body myositis, a myasthenia gravis, a polymyositis or any other type of inflammatory myopathy, as well as, a fasciitis, a fibrositis, a myositis, a neuromyotonia, a tendinosis, or a tendonitis. In addition, systemic administration could also be used to administer a disclosed TVEMP to treat chronic neurogenic inflammation.

A 73 year old female diagnosed with Churg-Strauss arteritis complains of wheezing when she breathes. A physician determines that the wheezing is due to chronic neurogenic inflammation. The woman is treated by systemically by intravenous administration of a composition comprising a TVEMP as disclosed in the present specification. The patient's condition is monitored and after about 1-3 days after treatment, and the woman indicates that she no longer is wheezing. At one and three month check-ups, the woman indicates that she still does not wheeze when she breathes. This reduction in chronic neurogenic inflammation symptoms indicates successful treatment with the composition comprising a TVEMP. A similar type of systemic administration of a TVEMP as disclosed in the present specification can be used to treat a patient suffering from chronic neurogenic inflammation associated with any vasculitis, such as, e.g., a Buerger's disease, a cerebral vasculitis, a cryoglobulinemia, an essential cryoglobulinemic vasculitis, a giant cell arteritis, a Golfer's vasculitis, a Henoch-Schonlein purpura, a hypersensitivity vasculitis, a Kawasaki disease, a microscopic polyarteritis/polyangiitis, a polyarteritis nodosa, a polymyalgia rheumatica (PMR), a rheumatoid vasculitis, a Takayasu arteritis, or a Wegener's granulomatosis, as well as, an arteritis, a carditis, an endocarditis, a heart disease, high blood pressure, an ischemic heart disease, a myocarditis, a pericarditis, a phlebitis, a pylephlebitis, or a thrombophlebitis.

A 37 year old male diagnosed with rosacea complains of skin redness. A physician determines that the redness is due to chronic neurogenic inflammation. The man is treated by local administration a composition comprising a TVEMP as disclosed in the present specification in the vicinity of the affected area. The patient's condition is monitored and after about 1-3 days after treatment, and the man indicates there is reduced skin redness. At one and three month check-ups, the man indicates that he continues to have improved skin tone and reduced redness This reduction in a chronic neurogenic inflammation symptom indicates successful treatment with the composition comprising a TVEMP. A similar type of local administration of a TVEMP as disclosed in the present specification can be used to treat a patient suffering from chronic neurogenic inflammation associated with an acne, a cervicitis, a dermatitis, an eczema (including an atopic eczema, a contact eczema, a xerotic eczema, a seborrhoeic dermatitis, a dyshidrosis, a discoid eczema, a venous eczema, a dermatitis herpetiformis, a neurodermatitis, or an autoeczematization), an endometritis, a gingivitis, a glossitis, a hidradenitis suppurativa, a keratitis, a keratoconjunctivitis, a mastitis, a psoriasis (including a plaqure psoriasis, a nail psoriasis, a guttate psoriasis, a scalp psoriasis, an inverse psoriasis, a pustular psoriasis, or an erythrodermis psoriasis), a scleroderma, a statis dermatitis, a stomatitis, a tonsillitis, a vaginitis, a vitiligo, or a vulvitis. In addition, systemic administration could also be used to administer a disclosed TVEMP to treat chronic neurogenic inflammation.

A 33 year old female diagnosed with Crohn's disease complains of abdominal pain and diarrhea. A physician determines that the abdominal pain and diarrhea is due to chronic neurogenic inflammation. The woman is treated by systemically by intravenous administration of a composition comprising a TVEMP as disclosed in the present specification. The patient's condition is monitored and after about 1-3 days after treatment, and the woman indicates that there is a reduction in abdominal pain and she no longer has diarrhea. At one and three month check-ups, the woman indicates that she continues to have reduced abdominal pain and diarrhea. This reduction in chronic neurogenic inflammation symptoms indicates successful treatment with the composition comprising a TVEMP. A similar type of systemic administration of a TVEMP as disclosed in the present specification can be used to treat a patient suffering from chronic neurogenic inflammation associated with any inflammatory bowel disease, such as, e.g., an ulcerative colitis (including ulcerative proctitis, left-sided colitis, pancolitis and fulminant colitis), any irritable bowel disease, as well as, a colitis, an enteritis, an enterocolitis, a gastritis, a gastroenteritis, a metabolic syndrome (syndrome X), a spastic colon, or any other gastrointestinal disorder.

A 46 year old male diagnosed with systemic lupus erythematosus complains of fever, joint pains, and fatigue. A physician determines that these symptoms are due to chronic neurogenic inflammation. The man is treated by systemically by intravenous administration a composition comprising a TVEMP as disclosed in the present specification. The patient's condition is monitored and after about 1-3 days after treatment, and the man indicates there is improvement in his health, his fever is gone, the pain in his joints is reduced and his is not as tired. At one and three month check-ups, the man indicates that he continues to have reduced joint pain and does not suffer from fevers or fatigue. This reduction in a chronic neurogenic inflammation symptom indicates successful treatment with the composition comprising a TVEMP. A similar type of systemic administration of a TVEMP as disclosed in the present specification can be used to treat a patient suffering from chronic neurogenic inflammation associated with any other systemic autoimmune disorder, including, without limitation, an anti-phospholipid antibody syndrome (APS), a bullous pemphigoid, a Chagas disease, a discoid lupus erythematosus, a drug-induced lupus erythematosus, a Goodpasture's syndrome, a Guillain-Barre syndrome, an idiopathic thrombocytopenic purpura, a myasthenia gravis, a neonatal lupus, a pernicious anemia, a polymyalgia rheumatica, a rheumatoid arthritis, a scleroderma, a Sjögren's syndrome, a subacute cutaneous lupus erythematosus, a Wegener's granulomatosis.

A 58 year old male diagnosed with Hashimoto's thyroiditis complains of depression, sensitivity to cold, weight gain, forgetfulness, and constipation. A physician determines that these symptoms are due to chronic neurogenic inflammation. The man is treated by local administration a composition comprising a TVEMP as disclosed in the present specification in the vicinity of the affected area. The patient's condition is monitored and after about 1-3 days after treatment, and the man indicates there is reduction in all the symptoms complained of. At one and three month check-ups, the man indicates that he still does not experience depression, sensitivity to cold, weight gain, forgetfulness, and constipation. This reduction in chronic neurogenic inflammation symptoms indicates successful treatment with the composition comprising a TVEMP. A similar type of systemic administration of a TVEMP as disclosed in the present specification can be used to treat a patient suffering from chronic neurogenic inflammation associated with any other local autoimmune disorder, including, without limitation, an acute disseminated encephalomyelitis (ADEM), an Addison's disease, an autoimmune hemolytic anemia, an autoimmune hepatitis (including primary biliary cirrhosis), an autoimmune inner ear disease, a celiac disease, a Crohn's disease, a diabetes mellitus type 1, an endometriosis, a giant cell arteritis, a Graves' disease, an interstitial cystitis, a lupus nephritis, a multiple sclerosis, a morphea, a pemphigus vulgaris, a recurrent disseminated encephalomyelitis, a sclerosing cholangitis, an ulcerative colitis, or a vitiligo. In addition, systemic administration could also be used to administer a disclosed TVEMP to treat chronic neurogenic inflammation.

A 59 year old male diagnosed with rheumatoid arthritis complains of joint stiffness and swelling. A physician determines that the joint stiffness and swelling is due to chronic neurogenic inflammation. The woman is treated by local administration a composition comprising a TVEMP as disclosed in the present specification in the vicinity of the affected area. The patient's condition is monitored and after about 1-3 days after treatment, and the woman indicates there is reduced joint stiffness and swelling. At one and three month check-ups, the woman indicates that she continues to have reduced joint stiffness and swelling in the area treated. This reduction in chronic neurogenic inflammation symptoms indicates successful treatment with the composition comprising a TVEMP. A similar type of local administration of a TVEMP as disclosed in the present specification can be used to treat a patient suffering from chronic neurogenic inflammation associated with any monoarthritis, oligoarthritis, or polyarthritis, such as, e.g., osteoarthritis, juvenile idiopathic arthritis, septic arthritis, a spondyloarthropathy (including ankylosing spondylitis, reactive arthritis (Reiter's syndrome), psoriatic arthritis, enteropathic arthritis associated with inflammatory bowel disease, Whipple disease or Behcet disease), a synovitis, gout, pseudogout, or Still's disease, as well as, a bursitis, a rheumatic fever, or a tenosynovitis. In addition, systemic administration could also be used to administer a disclosed TVEMP to treat chronic neurogenic inflammation.

In closing, it is to be understood that although aspects of the present specification have been described with reference to the various embodiments, one skilled in the art will readily appreciate that the specific examples disclosed are only illustrative of the principles of the subject matter disclosed in the present specification. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing quantities of ingredients, properties such as molecular weight, reaction conditions, and so forth used in the specification and claims are to be understood as being modified in all instances by the term “about.” As used herein, the term “about” when qualifying a value of a stated item, number, percentage, parameter, or term refers to a range of plus or minus ten percent of the value of the stated item, number, percentage, parameter, or term. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

All patents, patent publications, and other publications referenced and identified in the present specification are individually and expressly incorporated herein by reference in their entirety for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed:

1. A method of treating chronic neurogenic inflammation in a mammal, the method comprising the step of administering to the mammal in need thereof a therapeutically effective amount of a composition including a TVEMP comprising a retargeted peptide binding domain, a Clostridial toxin translocation domain and a Clostridial toxin enzymatic domain,
wherein the retargeted peptide binding domain is an interleukin (IL) peptide binding domain, a vascular endothelial growth factor (VEGF) peptide binding domain, an insulin-like growth factor (IGF) peptide binding domain, or an epidermal growth factor (EGF) peptide binding domain, and
wherein administration of the composition reduces the release of an inflammation inducing molecule, thereby reducing a symptom associated with chronic neurogenic inflammation.

2. The method of claim 1, wherein the TVEMP comprises a linear amino-to-carboxyl single polypeptide order of 1) the Clostridial toxin enzymatic domain, the Clostridial toxin translocation domain, the retargeted peptide binding domain, 2) the Clostridial toxin enzymatic domain, the retargeted peptide binding domain, the Clostridial toxin translocation domain, 3) the retargeted peptide binding domain, the Clostridial toxin translocation domain, and the Clostridial toxin enzymatic domain, 4) the retargeted peptide binding domain, the Clostridial toxin enzymatic domain, the Clostridial toxin translocation domain, 5) the Clostridial toxin translocation domain, the Clostridial toxin enzymatic domain and the retargeted peptide binding domain, or 6) the Clostridial toxin translocation domain, the retargeted peptide binding domain and the Clostridial toxin enzymatic domain.

3. The method of claim 1, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.

4. The method of claim 1, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.

5. A method of treating chronic neurogenic inflammation in a mammal, the method comprising the step of administering to the mammal in need thereof a therapeutically effective amount of a composition including a TVEMP comprising a retargeted peptide binding domain, a Clostridial toxin translocation domain, a Clostridial toxin enzymatic domain, and an exogenous protease cleavage site, wherein the retargeted peptide binding domain is an interleukin (IL) peptide binding domain, a vascular endothelial growth factor (VEGF) peptide binding domain, an insulin-like growth factor (IGF) peptide binding domain, or an epidermal growth factor (EGF) peptide binding domain, and wherein administration of the composition reduces the release of an inflammation inducing molecule, thereby reducing a symptom associated with chronic neurogenic inflammation.

6. The method of claim 5, wherein the TVEMP comprises a linear amino-to-carboxyl single polypeptide order of 1) the Clostridial toxin enzymatic domain, the exogenous protease cleavage site, the Clostridial toxin translocation domain, the retargeted peptide binding domain, 2) the Clostridial toxin enzymatic domain, the exogenous protease cleavage site, the retargeted peptide binding domain, the Clostridial toxin translocation domain, 3) the retargeted peptide binding domain, the Clostridial toxin translocation domain, the exogenous protease cleavage site and the Clostridial toxin enzymatic domain, 4) the retargeted peptide binding domain, the Clostridial toxin enzymatic domain, the exogenous protease cleavage site, the Clostridial toxin translocation domain, 5) the Clostridial toxin translocation domain, the exogenous protease cleavage site, the Clostridial toxin enzymatic domain and the retargeted peptide binding domain, or 6) the Clostridial toxin translocation domain, the exogenous protease cleavage site, the retargeted peptide binding domain and the Clostridial toxin enzymatic domain.

7. The method of claim 5, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.

8. The method of claim 5, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.

9. The method of claim 5, wherein the exogenous protease cleavage site is a plant papain cleavage site, an insect papain cleavage site, a crustacian papain cleavage site, an enterokinase cleavage site, a human rhinovirus 3C protease cleavage site, a human enterovirus 3C protease cleavage site, a tobacco etch virus protease cleavage site, a Tobacco Vein Mottling Virus cleavage site, a subtilisin cleavage site, a hydroxylamine cleavage site, or a Caspase 3 cleavage site.

10. Use of a TVEMP in the manufacturing a medicament for treating chronic neurogenic inflammation in a mammal in need thereof, wherein the TVEMP comprising a retargeted peptide binding domain, a Clostridial toxin translocation domain and a Clostridial toxin enzymatic domain, and an exogenous protease cleavage site, wherein the retargeted peptide binding domain is an interleukin (IL) peptide binding domain, a vascular endothelial growth factor (VEGF) peptide binding domain, an insulin-like growth factor (IGF) peptide binding domain, or an epidermal growth factor (EGF) peptide binding domain, and wherein administration of a therapeutically effective amount of the medicament to the mammal reduces the release of an inflammation inducing molecule, thereby reducing a symptom associated with chronic neurogenic inflammation.

11. Use of a TVEMP in the treatment of chronic neurogenic inflammation in a mammal in need thereof, the use comprising the step of administering to the mammal a therapeutically effective amount of the TVEMP, wherein the TVEMP comprising a retargeted peptide binding domain, a Clostridial toxin translocation domain, a Clostridial toxin enzymatic domain, and an exogenous protease cleavage site, wherein the retargeted peptide binding domain is an interleukin (IL) peptide binding domain, a vascular endothelial growth factor (VEGF) peptide binding domain, an insulin-like growth factor (IGF) peptide binding domain, or an epidermal growth factor (EGF) peptide binding domain, and wherein administration of the TVEMP reduces the release of an inflammation inducing molecule, thereby reducing a symptom associated with chronic neurogenic inflammation.

12. A method of treating chronic neurogenic inflammation in a mammal, the method comprising the step of administering to the mammal in need thereof a therapeutically effective amount of a composition including a TVEMP comprising a retargeted peptide binding domain, a Clostridial toxin translocation domain and a Clostridial toxin enzymatic domain,
wherein the retargeted peptide binding domain is an interleukin (IL) peptide binding domain, a vascular endothelial growth factor (VEGF) peptide binding domain, an insulin-like growth factor (IGF) peptide binding domain, or an epidermal growth factor (EGF) peptide binding domain, and
wherein administration of the composition reduces a symptom associated with chronic neurogenic inflammation, thereby treating chronic neurogenic inflammation.
